(19) United States
(12) Patent Application Publication
(10) Pub. No.: US 2024/0011012 A1
(43) Pub. Date: Jan. 11, 2024

(54) METHODS AND COMPOSITIONS FOR INHIBITING EXCESS NUCLEIC ACID PRECIPITATION

(71) Applicant: PFIZER INC., New York,, NY (US)

(72) Inventors: Neha KALLA, Morrisville, NC (US); William KISH, Morrisville, NC (US); John LIGHTHOLDER, Morrisville, NC (US); Zhuo LIU, Morrisville, NC (US); Eric VORST, Morrisville, NC (US); Tamara ZEKOVIC, Cary, NC (US)

(21) Appl. No.: 18/255,869

(22) PCT Filed: Dec. 17, 2021
(86) PCT No.: PCT/IB2021/061943

§ 371 Date: Jun. 4, 2023

Related U.S. Application Data
(60) Provisional application No. 63/199,368, filed on Dec. 21, 2020.
(60) Provisional application No. 63/263,924, filed on Nov. 11, 2021.

(51) Int. Cl. C12N 15/10; C12N 15/86

(52) U.S. Cl. CPC C12N 15/1003; C12N 15/86; C12N 2750/14143

(57) ABSTRACT

The present disclosure describes improved methods for use in purifying biological products made by host cells. In some embodiments, the improved methods comprise one or more steps of lysing host cells, such as with a detergent, to release the biological product, precipitating host cell DNA, such as with domiphen bromide, and then inhibiting precipitation of residual host cell DNA in a supernatant containing the biological product by adding a salt to a sufficient final concentration. In some embodiments, the biological product is a vaccine, or a viral vector for gene therapy, such as an AAV vector or a lentiviral vector.

### CROSS REFERENCE TO RELATED APPLICATIONS

This application claims the benefit of U.S. Provisional Application No. 63/199,368, filed Dec. 21, 2020, and U.S. Provisional Application No. 63/263,924, filed Nov. 11, 2021, the contents of each of which are hereby incorporated by reference in their entirety.

### BACKGROUND OF THE INVENTION

Various kinds of cells maintained in culture can serve as biological factories for making desired products. Such products include naturally occurring compounds made by unmodified cells, but cells can also be modified using genetic engineering technology to produce simple and more complex molecules and even supramolecular structures like viruses that such cells would ordinarily be unable to produce. Examples include transfecting bacteria with plasmids engineered to express relatively simple proteins, such as human growth hormone or insulin, or transforming eukaryotic cells, such as yeast, mammalian, or insect cells, with foreign genetic material capable of directing the cells to produce more complicated proteins, such as enzymes, clotting factors, and antibodies. This technology has enabled the efficient production of biological products with important medical and industrial applications that would not otherwise be possible.

Some biological products made by cells are secreted into the surrounding medium, from which the product can be purified directly, while the cells would typically be discarded. In other cases, however, desired biological products are not secreted, but instead are mostly or entirely retained within viable cells. Purification therefore requires disrupting the cell membranes in various ways, allowing the contents of the cells to spill out into the surrounding medium. While this approach is effective to release retained biological product from cells, it has the disadvantage of also releasing all the other cellular contents which must be removed in one or more downstream processing steps in order to comply with prevailing standards of purity for the product in question. For example, mammalian cells are used to produce many different kinds of recombinant biologic drugs, and the US Food and Drug Administration has issued guidance defining the maximum amount of cell-derived contaminants that may be present in drug products. Complying with these stringent standards can require complex purification schemes that are both inefficient in terms of product yield and expensive to implement.

One cell-derived contaminant that FDA seeks to limit is DNA, and different approaches have been developed to remove cell-derived DNA from crude cellular lysates. A common method is to add an endonuclease, such as Benzonase, to the lysate, which acts to digest cellular DNA into smaller fragments that can more easily be removed in downstream processing steps, or that may be undetectable in the drug product even if still present. Endonucleases are expensive reagents, however, and represent another biologically derived component in the lysate which must be removed downstream. Due to these disadvantages, other strategies for removing cell-derived DNA from crude lysates have been developed.

An approach for removing cellular DNA from lysates that does not rely on endonuclease is to add a cationic detergent, such as domiphen bromide (DB) in sufficient amount to precipitate the DNA. When allowed to settle out of solution as a flocculant mass, the precipitated DNA can readily be separated from the supernatant, which can then be processed further in one or more downstream purification steps. This approach enjoys the advantages of lower cost, as well as removal of a significant amount of host cell DNA before any downstream steps are implemented (endonuclease does not remove the DNA, but simply digests it into smaller fragments). The inventors observed, however, that residual cellular DNA and cationic detergent in the partially clarified supernatant can continue to react, forming small aggregates that remain in suspension and which can be detected as an increase in turbidity with time. If the precipitation reaction proceeds long enough, the suspended particles can foul chromatography columns used in downstream purification steps, reducing the number of performance cycles that a fresh column can undergo before the yield of biological product falls below an acceptable value. Accordingly, there exists a need in the art for improved methods for removing cellular DNA from crude cell lysates by precipitation with a cationic detergent, while preventing undesired precipitation of residual DNA in the clarified lysate by the detergent.

### SUMMARY OF THE INVENTION

The present disclosure addresses the need in the art by providing novel methods, compositions, and systems for removing host cell DNA and other contaminants from crude host cell lysates while inhibiting subsequent precipitation of residual host cell DNA that can interfere with downstream processing steps intended to purify a desirable biological product. According to certain non-limiting embodiments, such products include adeno-associated viral (AAV) vectors.

Those skilled in the art will recognize, or be able to ascertain using no more than routine experimentation, many equivalents of the specific embodiments of the disclosure described herein. Such equivalents are intended to be encompassed by the following embodiments (E).
E1. A method of removing host cell DNA from a sample of lysed host cells, comprising the steps of (i) lysing the host cells, producing a lysate, (ii) precipitating host cell DNA from the lysate, producing a flocculant and a supernatant (iii) separating the supernatant from the flocculant, (iv) inhibiting precipitation of residual host cell DNA in the supernatant, and optionally (v) purifying a biological product produced by the host cells.
E2. The method of E1, wherein the host cells are lysed mechanically, osmotically, chemically, or enzymatically.
E3. The method of E1 or E2, wherein the host cells are suspended in a physiologically compatible fluid, such as growth medium, and are lysed chemically by adding to the cell suspension a solution comprising a detergent in a concentration sufficient to cause cell lysis.
E4. The method of E1 or E2, wherein the host cells are grown or maintained as an adherent cell culture on a substrate and are lysed chemically by contacting them with a solution comprising a detergent in a concentration sufficient to cause cell lysis, or are first detached from their substrate and suspended in a physiologically compatible fluid to which the detergent solution is added.
E5. The method of E3 or E4, wherein the detergent is an ionic detergent, a non-ionic detergent, or a zwitterionic detergent.
E6. The method of E5, wherein the non-ionic detergent is selected from the group of detergent compounds consisting of alkylphenol ethoxylate, 4-alkylphenol ethoxylate, octylphenol ethoxylate, 4-octylphenol ethoxylate, nonylphenol ethoxylate, 4-nonylphenol ethoxylate, Triton X-100, Triton X-114, NP-40, Tween 20, and Tween 80.
E7. The method of E3 to E6, wherein prior to lysis, the viable cell density of the host cells in the physiologically compatible fluid is at least or about 8×10⁶, 9×10⁶, 10×10⁶, 11×10⁶, 12×10⁶, 13×10⁶, 14×10⁶, 15×10⁶, 16×10⁶, 17×10⁶, 18×10⁶, 19×10⁶, 20×10⁶, 21×10⁶, 22×10⁶, 23×10⁶, 24×10⁶, 25×10⁶, 26×10⁶, 27×10⁶, 28×10⁶, 29×10⁶, or 30×10⁶ viable cells per mL (vc/mL), or more, or a range including and between any two of the foregoing values, such as about 8×10⁶ to 15×10⁶ vc/mL, 10×10⁶ to 30×10⁶ vc/mL, 15×10⁶ to 25×10⁶ vc/mL, or 18×10⁶ to 22×10⁶ vc/mL.
E8. The method of E1 to E7, wherein the host cells are mammalian cells, such as HEK293 cells, CHO cells or HeLa cells, or insect cells, such as Sf9 cells or Sf1 cells.
E9. The method of E1 to E8, wherein the host cells are HEK293 cells.
E10. The method of E3 to E9, wherein the final concentration of detergent in the lysate is at least or about 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, 2.00%, 3.00%, 4.00%, or 5.00%, or a range of concentrations including and between any two of the foregoing values, such as from about 0.05% to 5.00%, 0.10% to 2.50%, 0.20% to 1.25%, 0.20% to 0.75%, 0.25% to 0.75%, 0.25% to 0.65%, 0.20% to 0.70%, 0.30% to 0.70%, 0.35% to 0.65%, 0.40% to 0.60%, or 0.45% to 0.55%.
E11. The method of E3 to E10, wherein the detergent is Triton X-100.
E12. The method of E11, wherein prior to lysis, the viable cell density of the host cells in the physiologically compatible fluid is at least about 10×10⁶ vc/mL and the final concentration of Triton X-100 in the lysate is at least about 0.30%.
E13. The method of E11, wherein prior to lysis, the viable cell density of the host cells in the physiologically compatible fluid is at least about 10×10⁶ vc/mL, and the final concentration of Triton X-100 in the lysate relative to the viable cell density prior to lysis is at least about 0.010% per 1×10⁶ vc/mL.
E14. The method of E11, wherein the final concentration of Triton X-100 in the lysate relative to the viable cell density prior to lysis is about 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.01% to 0.050% per 1×10⁶ vc/mL, 0.020% to 0.033% per 1×10⁶ vc/mL, 0.020% to 0.060% per 1×10⁶ vc/mL, 0.023% to 0.070% per 1×10⁶ vc/mL, 0.024% to 0.040% per 1×10⁶ vc/mL, or about 0.028% to 0.047% per 1×10⁶ vc/mL.
E15. The method of E3 to E14, wherein the volume of the cell suspension at the time of lysis is at least or about 2 liters (L), 5 L, 10 L, 20 L, 50 L, 100 L, 200 L, 500 L, 1000 L, 1500 L, 2000 L, or more, or a range including and between any of the foregoing values, such as 2 L to 100 L, 50 L to 500 L, 500 L to 1000 L, 500 L to 1500 L, 1000 L to 1500 L, or 500 L to 2000 L which, in some embodiments, is enclosed within a container, such as a tank, bag, or bioreactor.
E16. The method of E3 to E15, further comprising mixing the cell suspension and detergent solution which, in some embodiments, can be performed for at least or about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 180 mins, or a range of time including and between any two of the foregoing values.
E17. The method of E3 to E16, wherein host cell DNA in the lysate is precipitated by adding to the lysate a solution comprising an alkyl-dimethyl-(2-phenoxyethyl)azanium halide in a concentration sufficient to precipitate host cell DNA, wherein in some embodiments the alkyl-dimethyl-(2-phenoxyethyl)azanium halide is a domiphen halide, such as domiphen bromide, domiphen chloride, or domiphen iodide.
E18. The method of E17, wherein the final concentration of domiphen halide in the lysate is at least or about 0.05%, 0.10%, 0.15%, 0.20%, 0.25%, 0.30%, 0.35%, 0.40%, 0.45%, or 0.50%, or a range of concentrations including and between any two of the foregoing values, such as 0.10% to 0.50%, 0.10% to 0.40%, 0.10% to 0.30%, 0.10% to 0.20%, 0.15% to 0.45%, 0.20% to 0.50%, 0.20% to 0.40%, 0.20% to 0.30%, 0.25% to 0.35%, 0.25% to 0.45%, 0.30% to 0.50%, 0.30% to 0.40% or 0.40% to 0.50%.
E19. The method of E17 or E18, wherein prior to lysis, the viable cell density of the host cells in the physiologically compatible fluid is at least about 10×10⁶ vc/mL and the final concentration of domiphen halide in the lysate is at least about 0.20%.
E20. The method of E17 or E18, wherein prior to lysis, the viable cell density of the host cells in the physiologically compatible fluid is at least about 10×10⁶ vc/mL, and the final concentration of domiphen halide in the lysate relative to the viable cell density prior to lysis is at least 0.007% per 1×10⁶ vc/mL.
E21. The method of E17 or E18, wherein the final concentration of domiphen halide in the lysate relative to the viable cell density prior to lysis ranges from about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, or 0.020% to 0.033% per 1×10⁶ vc/mL.
E22. The method of E17 or E18, wherein prior to lysis, the viable cell density of the host cells in the physiologically compatible fluid is at least about 10×10⁶ vc/mL, the detergent is Triton X-100, the final concentration of Triton X-100 in the lysate is at least about 0.30%, and the final concentration of domiphen halide in the lysate is at least about 0.20%.
E23. The method of E17 or E18, wherein prior to lysis, the viable cell density of the host cells in the physiologically compatible fluid is at least about 10×10⁶ vc/mL, the detergent is Triton X-100, the final concentration of Triton X-100 in the lysate relative to the viable cell density prior to lysis is at least about 0.010% per 1×10⁶ vc/mL, and the final concentration of domiphen halide in the lysate relative to the viable cell density prior to lysis is at least 0.007% per 1×10⁶ vc/mL.
E24. The method of E17 or E18, wherein the final concentration of domiphen halide in the lysate relative to the viable cell density prior to lysis is not less than 0.009%, 0.008%, or 0.007% per 1×10⁶ vc/mL.
E25. The method of E17, wherein the final concentration of Triton X-100 in the lysate ranges from about 0.3% to 0.7% and the ratio of the concentration of domiphen halide to the concentration of Triton X-100 in the lysate ranges from about 0.100 to 0.333, 0.150 to 0.500, 0.200 to 0.667, 0.250 to 0.833, 0.300 to 1.000, 0.350 to 1.167, 0.400 to 1.333, 0.450 to 1.500, 0.500 to 1.667, 0.550 to 1.833, 0.600 to 2.000, 0.100 to 0.250, 0.150 to 0.375, 0.200 to 0.500, 0.250 to 0.625, 0.300 to 0.750, 0.350 to 0.875, 0.400 to 1.000, 0.450 to 1.125, 0.500 to 1.250, 0.550 to 1.375, 0.600 to 1.500, 0.100 to 0.200, 0.150 to 0.300, 0.200 to 0.400, 0.250 to 0.500, 0.300 to 0.600, 0.350 to 0.700, 0.400 to 0.800, 0.450 to 0.900, 0.500 to 1.000, 0.550 to 1.100, 0.600 to 1.200, 0.100 to 0.167, 0.150 to 0.250, 0.200 to 0.333, 0.250 to 0.417, 0.300 to 0.500, 0.350 to 0.583, 0.400 to 0.667, 0.450 to 0.750, 0.500 to 0.833, 0.550 to 0.917, 0.600 to 1.000, 0.100 to 0.143, 0.150 to 0.214, 0.200 to 0.286, 0.250 to 0.357, 0.300 to 0.429, 0.350 to 0.500, 0.400 to 0.571, 0.450 to 0.643, 0.500 to 0.714, 0.550 to 0.786, or 0.600 to 0.857.
E26. The method of E17, wherein the final concentration of Triton X-100 in the lysate ranges from about 0.4% to 0.6% and the ratio of the concentration of domiphen halide to the concentration of Triton X-100 in the lysate ranges from about 0.150 to 0.375, 0.200 to 0.500, 0.250 to 0.625, 0.300 to 0.750, 0.350 to 0.875, 0.400 to 1.000, 0.450 to 1.125, 0.150 to 0.300, 0.200 to 0.400, 0.250 to 0.500, 0.300 to 0.600, 0.350 to 0.700, 0.400 to 0.800, 0.450 to 0.900, 0.150 to 0.250, 0.200 to 0.333, 0.250 to 0.417, 0.300 to 0.500, 0.350 to 0.583, 0.400 to 0.667, or 0.450 to 0.750.
E27. The method of E17, wherein the final concentration of Triton X-100 in the lysate is about 0.4% and the ratio of the concentration of domiphen halide to the concentration of Triton X-100 in the lysate ranges from about 0.150 to 0.375, 0.200 to 0.500, 0.250 to 0.625, 0.300 to 0.750, to 0.875, 0.400 to 1.000, or 0.450 to 1.125; or the final concentration of Triton X-100 in the lysate is about 0.5% and the ratio of the concentration of domiphen halide to the concentration of Triton X-100 in the lysate ranges from about 0.150 to 0.300, 0.200 to 0.400, 0.250 to 0.500, 0.300 to 0.600, 0.350 to 0.700, 0.400 to 0.800, or 0.450 to 0.900; or the final concentration of Triton X-100 in the lysate is about 0.6% and the ratio of the concentration of domiphen halide to the concentration of Triton X-100 in the lysate ranges from about 0.150 to 0.250, 0.200 to 0.333, 0.250 to 0.417, 0.300 to 0.500, 0.350 to 0.583, 0.400 to 0.667, or 0.450 to 0.750.
E28. The method of E17, E18 and E22, wherein the viable cell density prior to lysis ranges from about 10×10⁶ vc/mL to 30×10⁶ vc/mL or 15×10⁶ vc/mL to 25×10⁶ vc/mL, the detergent is Triton X-100, the final concentration of Triton X-100 in the lysate ranges from about 0.35% to 0.65% or 0.4% to 0.6%, and the final concentration of domiphen halide in the lysate ranges from about 0.15% to 0.45%, 0.2% to 0.3%, or 0.2% to 0.4%.
E29. The method of E17, E18 and E22, wherein the viable cell density prior to lysis ranges from about 15×10⁶ vc/mL to 25×10⁶ vc/mL, the detergent is Triton X-100, the final concentration of Triton X-100 in the lysate ranges from about 0.4% to 0.6%, and the final concentration of domiphen halide in the lysate ranges from about 0.2% to 0.3% or 0.2% to 0.4%.
E30. The method E17, E18 and E22, wherein the viable cell density prior to lysis ranges from about 15×10⁶ vc/mL to 25×10⁶ vc/mL, the final concentration of Triton X-100 in the lysate is about 0.5%, and the final concentration of domiphen halide in the lysate ranges from about 0.2% to 0.3% or 0.2% to 0.4%.
E31. The method of E1 to E30, wherein the host cells produce an AAV vector and the amount of residual host cell DNA in drug substance comprising the AAV vector purified from the supernatant is less than about 100, 90, 80, 70, 60, or 50 picograms per 1×10⁹ vector genomes (pg/1×10⁹ vg).
E32. The method of E17 to E31, wherein the alkyl-dimethyl-(2-phenoxyethyl)azanium halide is domiphen bromide (DB).
E33. The method of E17 to E32, further comprising mixing the lysate and the domiphen halide solution which, in some embodiments, can be performed for at least or about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 180 mins, or a range of time including and between any two of the foregoing values.
E34. The method of E1 to E33, wherein the supernatant is separated from the flocculant by allowing the flocculant to settle under the influence of gravity to the bottom of a container holding the lysate, forming a lower layer of settled flocculant and an upper layer of supernatant.
E35. The method of E34, wherein the flocculant is allowed by settle for at least or about 0.5 hr, 1 hr, 1.5 hrs, 2 hrs, 2.5 hrs, 3 hrs, 3.5 hrs, 4 hrs, 5 hrs, 6 hrs, 7 hrs, 8 hrs, 9 hrs, 10 hrs, 11 hrs, 12 hrs, or a range of time including and between any two of the foregoing values.
E36. The method of E1 to E33, wherein the supernatant is separated from the flocculant by centrifuging a container holding the lysate, forming a pellet of flocculant which is separate from the supernatant.
E37. The method of E34 to E36, wherein the supernatant is removed from the container, such as by pumping, leaving the flocculant in the container.
E38. The method of E37, wherein after removing the supernatant, the supernatant is filtered, such as by depth filtration, which in some embodiments can be performed using a depth filter having a nominal retention rating of less than or equal to about 100 μm, 50 μm, 40 μm, 30 μm, 25 μm, 20 μm, 15 μm, 10 μm, 5 μm, 2 μm, 1 μm, or 0.5 μm.
E39. The method of E17 to E38, wherein precipitation of residual host cell DNA in the supernatant by the domiphen halide is inhibited by adding to the supernatant a solution comprising a salt in a concentration sufficient to inhibit precipitation of host cell DNA by domiphen halide.
E40. The method of E39, wherein the salt solution further comprises a detergent, such as Triton X-100.
E41. The method of E39, wherein the salt is sodium chloride (NaCl), potassium chloride (KCl), magnesium sulfate (MgSO₄), or magnesium chloride (MgCl₂).
E42. The method of E39 to E41, wherein the final concentration of the added salt in the supernatant is at least or about 1 mM, 5 mM, 10 mM, 20 mM, 30 mM, 40 mM, 50 mM, 75 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 500 mM, 550 mM, 600 mM, 650 mM, 700 mM, 750 mM, or 800 mM.
E43. The method of E39 to E42, wherein prior to lysis, the viable cell density of the host cells in the physiologically compatible fluid is at least about 10×10⁶ vc/mL, the final concentration of domiphen halide in the lysate is at least about 0.20%, the salt is NaCl or KCl, and the final concentration of the added salt in the supernatant is at least about 100 mM.
E44. The method of E39 to E42, wherein prior to lysis, the viable cell density of the host cells in the physiologically compatible fluid is at least about 10×10⁶ vc/mL, the final concentration of domiphen halide in the lysate is at least about 0.20%, the salt is MgSO₄ or MgCl₂, and the final concentration of the added salt in the supernatant is at least about 10 mM.
E45. The method of E39 to E42, wherein prior to lysis, the viable cell density of the host cells in the physiologically compatible fluid is at least 10×10⁶ vc/mL, the final concentration of domiphen halide in the lysate relative to the viable cell density prior to lysis is at least 0.007% per 1×10⁶ vc/mL; and (i) the salt is NaCl or KCl, and the final concentration of the added salt in the supernatant is at least about 100 mM, or (ii) the salt is MgSO₄ or MgCl₂, and the final concentration of the added salt in the supernatant is at least about 10 mM.
E46. The method of E43 to E45, wherein the final concentration of domiphen halide in the lysate relative to the viable cell density prior to lysis is not less than 0.007% per 1×10⁶ vc/mL.
E47. The method of E43 or E44, wherein the viable cell density prior to lysis ranges from about 10×10⁶ vc/mL to 30×10⁶ vc/mL.
E48. The method of E43 or E44, wherein the viable cell density prior to lysis ranges from about 15×10⁶ vc/mL to 25×10⁶ vc/mL.
E49. The method of E43, E44, E47, or E48, wherein the final concentration of domiphen halide in the lysate ranges from about 0.2% to 0.5%, 0.2% to 0.4%, or 0.2% to 0.3%.
E50. The method of E43 or E44, or E47 to E49, wherein the detergent is Triton X-100 and the final concentration of Triton X-100 in the lysate is at least about 0.30%.
E51. The method of E43 or E44, or E47 to E50, wherein the detergent is Triton X-100 and the final concentration of Triton X-100 in the lysate ranges from about 0.3% to 0.7%, 0.35% to 0.65%, 0.4% to 0.6%, or is about 0.5%.
E52. The method of E43 or E44, or E47 to E51, wherein the viable cell density prior to lysis ranges from about 15×10⁶ vc/mL to 25×10⁶ vc/mL, the concentration of Triton X-100 in the lysate is about 0.5%, and the concentration of domiphen halide in the lysate ranges from about 0.2% to 0.3%, or 0.2% to 0.4%.
E53. The method of E39 to E52, wherein the salt is NaCl or KCl, and the final concentration of the added salt in the supernatant ranges from about 100 mM to 300 mM, 100 mM to 350 mM, 100 mM to 400 mM, 100 mM to 450 mM, 100 mM to 500 mM, 100 mM to 550 mM, 100 mM to 600 mM, 100 mM to 650 mM, 100 mM to 700 mM, 100 mM to 750 mM, 100 mM to 800 mM, 150 mM to 300 mM, 150 mM to 350 mM, 150 mM to 400 mM, 150 mM to 450 mM, 150 mM to 500 mM, 150 mM to 550 mM, 150 mM to 600 mM, 150 mM to 650 mM, 150 mM to 700 mM, 150 mM to 750 mM, 150 mM to 800 mM, 200 mM to 300 mM, 200 mM to 350 mM, 200 mM to 400 mM, 200 mM to 450 mM, 200 mM to 500 mM, 200 mM to 550 mM, 200 mM to 600 mM, 200 mM to 650 mM, 200 mM to 700 mM, 200 mM to 750 mM, 200 mM to 800 mM, 250 mM to 300 mM, 250 mM to 350 mM, 250 mM to 400 mM, 250 mM to 450 mM, 250 mM to 500 mM, 250 mM to 550 mM, 250 mM to 600 mM, 250 mM to 650 mM, 250 mM to 700 mM, 300 mM to 400 mM, 300 mM to 450 mM, 300 mM to 500 mM, 300 mM to 550 mM, 300 mM to 600 mM, 300 mM to 650 mM, 300 mM to 700 mM, 350 mM to 400 mM, 350 mM to 450 mM, 350 mM to 500 mM, 350 mM to 550 mM, 350 mM to 600 mM, 350 mM to 650 mM, 350 mM to 700 mM, 400 to 500 mM, 400 to 550 mM, 400 to 600 mM, 400 to 650 mM, 450 to 500 mM, 450 to 550 mM, 450 to 600 mM, or 450 to 650 mM.
E54. The method of E39 to E53, further comprising mixing the supernatant and salt solution which, in some embodiments, can be performed for at least or about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 180 mins, or a range of time including and between any two of the foregoing values.
E55. The method of E39 to E54, wherein the period of time between the steps of separating the supernatant and adding the salt solution to the supernatant for inhibiting precipitation of residual host cell DNA is less than about 12 hrs, 6 hrs, 3 hrs, 2 hrs, 1 hr, 45 mins, 30 mins, 15 mins, 10 mins, 5 mins, or less.
E56. The method of E54, wherein the supernatant and the salt solution are mixed in a batch.
E47. The method of E54, wherein the supernatant and the salt solution are mixed continuously.
E58. The method of E54, further comprising filtering the mixture of the supernatant and salt solution.
E59. The method of E58, wherein the filtering is performed using a membrane filter having an average pore size of less than or equal to about 10 μm, 5 μm, 2 μm, 1 μm, 0.5 μm, 0.2 μm, or 0.1 μm.
E60. The method of E54 to E59, further comprising purifying the biological product from the mixture of the supernatant and salt solution in at least one downstream processing step.
E61. The method of E60, wherein the period of time between the steps of adding the salt solution to the supernatant and purifying the biological product is less than about 72 hrs, 48 hrs, 36 hrs, 24 hrs, 12 hrs, 9 hrs, 6 hrs, 3 hrs, 2 hrs, 90 mins, 60 mins, 45 mins, 30 mins, 15 mins, or 10 mins, or less.
E62. The method of E1 to E61, wherein the biological product produced by the host cells is recombinant.
E63. The method of E1 to E61, wherein the biological product produced by the host cells is a virus particle.
E64. The method of E63, wherein the virus particle is an adenovirus particle, an adeno-associated virus (AAV) particle, a retrovirus particle, or a lentivirus particle.
E65. The method of E63 or E64, wherein the virus particle is modified to express a heterologous gene, forming a viral vector.
E66. The method of E65, wherein the viral vector is an AAV vector.
E67. The method of E66, wherein the AAV vector comprises a capsid that binds more strongly to sialic acid or galactose as compared to HSPG.
E68. The method of E66 or E67, wherein the AAV vector comprises an AAV1, AAV4, AAV5, or AAV9 capsid.
E69. The method of E60 or E61, wherein the downstream processing step comprises chromatography.
E70. The method of E69, wherein the chromatography is affinity chromatography, immunoaffinity chromatography, pseudoaffinity chromatography, anion exchange chromatography, cation exchange chromatography, hydrophobic interaction chromatography, or size exclusion chromatography.
E71. The method of E69 or E70, wherein the biological product is an AAV vector, and the method is effective to achieve an AAV vector yield of at least 50%, 60%, 70%, 80%, 90%, or 100% after at least 5, 6, 7, 8, 9, 10, 11, 12, 13, 14, 15 or more chromatography purification cycles.
E72. The method of E71, wherein the method is effective to achieve an AAV vector yield of at least 50% after at least 5 chromatography purification cycles.
E73. The method of E70 to E72, wherein the chromatography is affinity chromatography.
E74. The method of E73, wherein the chromatography is immunoaffinity chromatography.
E75. The method of E71 to E74, wherein the AAV vector comprises a capsid that binds more strongly to sialic acid or galactose as compared to HSPG.
E76. The method of E71 or E74, wherein the AAV vector comprises an AAV1, AAV4, AAV5, or AAV9 capsid.
E77. A biological product produced by the method of E1 to E76.
E78. The biological product of E77, wherein said biological product is a viral vector.
E79. The biological product of E77, wherein said biological product is an AAV vector.
E80. A composition comprising a biological product produced by the method of E1 to E76.
E81. The composition of E80, wherein said biological product a viral vector.
E82. The composition of E80, wherein said biological product is an AAV vector.
E83. The composition of E82, wherein the capsids of the AAV vector in said composition are at least 20%, 30%, 40%, 50%, 60%, or 70% full.
E84. The composition of E82 or E83, wherein said composition comprises not more than about 200, 150, 100, 90, 80, 70, 60, 50, 45, 40, 35, 30, 25, or 20 pg/1×10⁹ vg of host cell DNA.
E85. A mixture of a supernatant and a salt solution produced by the method of E39 to E59, wherein the turbidity of the mixture is not more than about 150, 100, 50, 40, 30, 20, 10, or 5 nephelometric turbidity units (NTUs).
E86. A system for performing the method of any of E39 to E59, said system comprising: (i) means for containing the supernatant, (ii) means for containing the salt solution, and (iii) means for mixing the supernatant and salt solution.
E87. The system of E86, further comprising means for fluid communication from the respective container means to the mixing means.
E88. The system of E87, further comprising means for pumping supernatant and salt solution from their respective container means through the fluid communication means.
E89. The system of E86 to E88, wherein said mixing means is a static in-line mixing means.

### BRIEF DESCRIPTION OF THE DRAWINGS

FIG. 1 illustrates the concentration dependence with which domiphen bromide (DB) precipitates host cell DNA (HC-DNA) from detergent lysed HEK293 cells transfected to produce an AAV9 based vector. Final DB concentration (% w/w) in lysate is indicated on the x-axis. Vector titer in genomes per mL is indicated on the left y-axis, and the concentration of host cell DNA normalized as nanograms per 1E14 VG is indicated in the right y-axis. Both host cell DNA and vector were quantified by qPCR. To generate trend lines, individual data points were fitted to a polynomial equation for the vector titer, and to an exponential equation for the host cell DNA concentration.

FIG. 2A illustrates the concentration dependence with which domiphen bromide (DB) precipitates host cell DNA (HC-DNA) from detergent lysed HEK293 cells transfected to produce an AAV9 based vector. Final DB concentration (% w/w) in lysate is indicated on the x-axis. Vector titer in genomes per mL is indicated on the left y-axis, and the concentration of host cell DNA normalized as picograms per VG is indicated in the right y-axis. Both host cell DNA and vector were quantified by qPCR. To generate trend lines, individual data points were fitted to linear equations. FIG. 2B illustrates the concentration dependence with which Triton X-100 lysed HEK293 cells transfected to produce an AAV9 based vector releases host cell DNA and vector into the lysate. Final Triton X-100 concentration (% w/w) in lysate is indicated on the x-axis. Vector titer in genomes per mL is indicated on the left y-axis, and the concentration of host cell DNA normalized as picograms per VG is indicated in the right y-axis. Both host cell DNA and vector were quantified by qPCR. To generate trend lines, individual data points were fitted to linear equations.

FIG. 3 illustrates the time and concentration dependence with which NaCl inhibits ongoing precipitation by DB of residual host cell DNA in clarified lysates of HEK293 cells transfected to produce an AAV9 based vector. Precipitation was detected as an increase in turbidity with time and expressed in nephelometric turbidity units (NTUs).

FIG. 4 illustrates the time and concentration dependence with which NaCl inhibits ongoing precipitation by DB of residual host cell DNA in clarified lysates of HEK293 cells transfected to produce an AAV9 based vector. Precipitation was detected as an increase in turbidity with time and expressed in nephelometric turbidity units (NTUs).

FIG. 5A illustrates the time and concentration dependence with which NaCl inhibits ongoing precipitation by DB of residual host cell DNA in clarified lysates of HEK293 cells transfected to produce an AAV9 based vector. Precipitation was detected as an increase in turbidity with time and expressed in nephelometric turbidity units (NTUs). FIG. 5B illustrates the time and concentration dependence with which MgSO₄, and salts comprising sodium anion and different inorganic and organic cations, inhibit ongoing precipitation by DB of residual host cell DNA in clarified lysates of HEK293 cells transfected to produce an AAV9 based vector. Precipitation was detected as an increase in turbidity with time and expressed in nephelometric turbidity units (NTUs). FIG. 5C illustrates the time and concentration dependence with which glycine and salts comprising chloride and different anions, inhibit ongoing precipitation by DB of residual host cell DNA in clarified lysates of HEK293 cells transfected to produce an AAV9 based vector. Precipitation was detected as an increase in turbidity with time and expressed in nephelometric turbidity units (NTUs). FIG. 5D illustrates the time and concentration dependence with which the most potent salts inhibit ongoing precipitation by DB of residual host cell DNA in clarified lysates of HEK293 cells transfected to produce an AAV9 based vector. Precipitation was detected as an increase in turbidity with time and expressed in nephelometric turbidity units (NTUs).

FIGS. 6A, 6B, 6C, and 6D illustrate the relationship between time and ionic strength (as measured by conductivity), and the inhibition of ongoing precipitation by DB of residual host cell DNA in clarified lysates of HEK293 cells transfected to produce an AAV9 based vector. Precipitation was detected as an increase in turbidity with time and expressed in nephelometric turbidity units (NTUs). Conductivity for multiple salts and their effect on turbidity was determined on the Day 0 of the experiment (i.e., the first day) (data shown in FIG. 6A), and on subsequent Day 1 (data shown in FIG. 6B), Day 2 (data shown in FIG. 6C), and Day 3 (data shown in FIG. 6D).

FIG. 7A illustrates a system for continuously mixing a precipitation inhibitor, here exemplified as a solution comprising 4 M NaCl and 0.5% Triton X-100, with clarified lysate from host cells lysed with detergent and treated with an agent to precipitate host cell DNA. FIG. 7B illustrates a system for batch mixing a precipitation inhibitor, here exemplified as a solution comprising 4 M NaCl and 0.5% Triton X-100, with clarified lysate from host cells lysed with detergent and treated with an agent to precipitate host cell DNA. FIG. 7C illustrates a system for continuously mixing a precipitation inhibitor, here exemplified as a solution comprising 4 M NaCl and 0.5% Triton X-100, with clarified lysate from host cells lysed with detergent and treated with an agent to precipitate host cell DNA.

FIG. 8 is a chromatogram illustrating performance of an immunoaffinity chromatography column over five cycles purifying an AAV9 based vector from clarified cellular lysates that had not been salt treated to inhibit precipitation of residual host cell DNA by domiphen bromide.

### DETAILED DESCRIPTION OF THE INVENTION

Described in detail below are exemplary non-limiting embodiments of various methods, compositions, and systems which can usefully be employed to remove host cell DNA from crude host cell lysates without resort to use of endonucleases, such as Benzonase. Certain of these embodiments offer the further advantage of inhibiting precipitation of residual host cell DNA in clarified lysates, which have been observed to reduce the efficiency of downstream steps intended to at least partially purify a desired biological product made by the host cells, an example of which are adeno-associated viral (AAV) vectors. Although an advantage of the methods described herein is effective removal of host cell DNA without use of exogenously added endonucleases, use of such enzymes if desired is not foreclosed in certain embodiments of the methods.

According to certain embodiments, the disclosure provides methods of lysing a preparation or sample of host cells which have produced a desired biological product, precipitating at least a portion of the host cell DNA released from the cells to form a flocculant and a supernatant, separating at least some of the supernatant from the flocculated host cell DNA, and inhibiting precipitation of residual host cell DNA in the supernatant. In some embodiments, before host cells are lysed, they are grown or maintained in culture for time and under conditions sufficient to produce the desired biological product. In other embodiments, after precipitation of residual host cell DNA is inhibited, the desired biological product is at least partially purified from the supernatant.

Host Cells

As used herein, “host cells” means cells suitable for or adapted to in vitro production of desired biological products. Host cells are often clonal cell lines capable of dividing for multiple generations before senescence stops growth, or may even be immortal. For use in the methods of the disclosure, host cells can be modified, transiently or non-transiently, through the introduction of exogenous genetic information designed to direct biosynthesis in host cells of specific biological products. For example, host cells can be transfected with nucleic acid containing a nucleobase sequence encoding a protein or regulatory RNA (such as lncRNA, miRNA, or siRNA). In some embodiments, the nucleic acid is DNA, such as a plasmid in which the coding sequence is under the control of a transcriptional regulatory element, such as a promoter and enhancer, that can be acted on by the cellular transcription and splicing machinery to produce mRNA. In other embodiments, nucleic acid can be RNA, such as mRNA, capable of being directly translated into protein.

Various ways are known in the art for transfecting host cells with DNA or RNA. These include, without limitation, mixing DNA or RNA with certain compounds that can complex with nucleic acids and then be taken up into the cells, including calcium phosphate or cationic organic compounds, such as DEAE-dextran, polyethylenimine (PEI), polylysine, polyornithine, polybrene, cyclodextrin, cationic lipids, and others known in the art. Transfection can also be performed non-chemically via electroporation and more exotic technologies, such as biolistic particle delivery. As known in the art, transfection can be transient or stable. With transient transfection, the transfected DNA or RNA exists in the cell for a limited period of time and, in the case of DNA, does not integrate into the genome. With stable transfection, DNA introduced into the cell can persist for long periods either as an episomal plasmid, or integrated into a chromosome. Usually, to produce stably transfected cells, a plasmid containing a selection marker, as well as the gene or genes for expressing the desired biological product, is transfected into the cells which are then grown and maintained under selective pressure, i.e., conditions that kill non-transfected cells or transfected cells from which the exogenous DNA, including its selection marker, are lost for some reason. For example, plasmids can contain an antibiotic resistance gene and transfected cells can be selected for by adding the antibiotic to the media in which the cells are grown. In some embodiments, the gene for producing the biological product introduced into stably transfected host cells is under the control of an inducible promoter and is not expressed, or only at a low level, unless an environmental factor, such as a drug, metal ion, or temperature increase, which induces the promoter, is introduced as the cells are grown.

In other embodiments, host cells genomes can be modified in a non-transient and targeted fashion using genetic engineering methods, such as knock-in, or gene editing methods, to direct host cells to produce desired biological products, components thereof, or other gene products necessary for the biosynthesis of such products. The invention is not limited by the manner in which host cells are generated. Foreign genes can also be introduced into host cells for purposes of directing production of desired biological products by transduction, in which host cells are infected with modified viruses (i.e., vectors) containing such genes. Examples of viral vectors useful for such purposes include adenovirus, retroviruses (including lentiviruses), baculoviruses, vaccinia virus, and herpes simplex virus, with others being possible.

Host cells can be any type of cell known in the art to be useful for the purpose of biosynthesizing desired biological products. Host cells can be prokaryotic cells, such as bacteria, such as E. coli, or eukaryotic cells, such as fungal cells, such as yeast cells, such as plant cells, or such as animal cells, such as insect cells or mammalian cells, including rat, mouse, or human cells. In some embodiments, host cells useful in the methods of the disclosure are mammalian host cells, examples of which include HeLa cells, COS cells, HEK293 cells (and variants of HEK293 cells, such as HEK293E, HEK293F, HEK293H, HEK293T or HEK293FT cells), A549 cells, BHK cells, Vero cells, NIH 3T3 cells, HT-1080 cells, Sp2/0 cells, NS0 cells, C127 cells, AGE1.HN cells, CAP cells, HKB-11 cells, or PER.C6 cells, with many others being possible. In some embodiments, host cells useful in the methods of the disclosure are insect host cells, examples of which include Sf9 cells, ExpiSf9, Sf21 cells, S2 cells, D.Mel2 cells, Tn-368 cells, or BTI-Tn-5B1-4 cells, with many others being possible.

For purposes of producing biological products, host cells are often grown or maintained in culture under controlled conditions conducive to their growth to relatively high density and the biosynthesis of the desired biological product. For example, host cells can be grown in liquid media of defined chemical composition that provides all the nutrients necessary for cell growth and biosynthesis. Exemplary media includes DMEM, DMEM/F12, MEM, RPMI 1640, for mammalian host cells, and Express Five SFM, Sf-900 II SFM, Sf-900 III, or ExpiSf CD, for certain insect cells. Such media may be supplemented with antibiotics, growth factors or cytokines (produced recombinantly or present in animal serum, such as FBS) known to stimulate growth of the particular type of cells in use, as well as other ingredients that may be required for optimal biosynthesis and/or activity of a desired biological product, but that would otherwise be in limiting supply. Exemplary supplements include essential amino acids, glutamine, vitamin K, insulin, BSA, or transferrin. In addition to the growth media, other culture conditions may be controlled to optimize growth and/or productivity of the cells, such as pH, temperature and CO₂ and oxygen concentration.

Host cells in culture can be grown or maintained in many containers known in the art, such as stirred tank bioreactors, wave bags, spinner flasks, hollow fiber bioreactors, or roller bottle, some of which can be designed and configured for single use or multiple use. Depending on the characteristics of the host cells in question, host cells can be grown in adherent cell culture, where the cells attach to and grow while in contact with a physical substrate, or in suspension cell culture, either where single cells float free in the media that sustains them, or while attached to bead microcarriers, which are suspended in the media. As known in the art, various technologies have been developed and can be used to grow host cells to high cell density, such as perfusion culture.

As known in the art, samples of host cells are often maintained in frozen cell banks, such as master cell banks and working cell banks, which facilitate production of biological products in many batches over time, while ensuring consistent performance by the host cells. Before a campaign to produce a biological product, a frozen sample of host cells from a cell bank would typically be thawed, seeded into a small culture volume, and grown to ever higher densities or numbers in cultures of increasing volume. When host cells have reached a desired cell density and/or volume in culture, exogenous genetic material can be introduced, such as by transfection with plasmid DNA or infection with viral vectors, to cause them to begin producing the desired biological product. Or, if using non-transiently modified host cells in which the genes for the biological product are under inducible control, the environmental factor necessary to induce expression can be introduced. Host cells can then be grown or maintained in culture for time and under conditions sufficient for them to produce a desired amount of the biological product.

Biological Products

The methods of the disclosure can usefully be employed in the production of any biological product capable of being made by a host cell, a significant portion of which is retained within host cells with intact cell membranes. Non-limiting examples include recombinant proteins of any kind, including monoclonal antibodies of any type and specificity, clotting factors, enzymes (whether for use as therapeutics or in industrial applications), growth factors, hormones, cytokines, antigenic proteins to serve as vaccines, and any naturally occurring or non-naturally occurring versions or variants of any of the foregoing, including versions that are fused with heterologous protein regions or domains, such as fusion of a clotting factor with albumin, or an Fc region from an immunoglobulin protein. Such proteins can include any post-translational modification known to those of skill in the art, such as covalent addition of carbohydrate groups, lipid molecules, and non-standard amino acids. Such proteins can also comprise a plurality of polypeptide chains, which can be covalently or non-covalently bound to each other. In other embodiments, biological products can be supramolecular assemblies, such as viruses, or modified viruses engineered to kill cancer cells (oncolytic viruses) or to serve as vectors of heterologous genes, for example as vectors to be used in gene therapy. Non-limiting examples include adenovirus, vaccinia virus, lentiviruses, and adeno-associated viruses, or vectors made using such viruses.

Adeno-Associated Viral (AAV) Vectors

According to some embodiments, the methods of the disclosure are useful in the production of adeno-associated virus (AAV) which has been recombinantly modified to function as a viral vector for gene therapy (thus, an AAV vector). So modified, AAV vectors are capable of delivering gene cassettes, often including regulatory elements for the appropriate initiation and termination of gene transcription, into targeted cells via transduction. In this way, AAV vectors can supply a functional copy of a gene to a target cell in which the endogenous version is missing or mutated.

As is well known in the art, AAV is a small non-enveloped, apparently non-pathogenic virus that depends on certain other viruses to supply gene products, known as helper factors, essential to its own replication, a quirk of biology that has made AAV well-suited to serve as a recombinant vector. For example, adenovirus (AdV) can serve as a helper virus by providing certain adenoviral factors, such as the E1A, E1B55K, E2A, and E4ORF6 proteins, and the VA RNA, in cells co-infected by adenovirus and AAV. Numerous types of AAV have been discovered which are restricted in their ability to infect certain animals (such as mammal and bird) and species (such as human and rhesus monkey), and having a tendency within species to infect certain tissues (such as liver or muscle) more so than others, a phenomenon called tropism, based on specific binding to different cell surface receptors. One type of AAV that infects humans, called AAV2, is particularly well characterized biologically, although many other types have found utility in creating gene therapy vectors.

In nature, the AAV genome is a single strand of DNA, about 4.7 kilobases long in AAV2, which contains two genes called rep and cap. By virtue of alternative splicing of the transcripts from two promoters, the rep gene produces four related multifunctional proteins called Rep (Rep 78, Rep 68, Rep 52 and Rep 40 in AAV2) which are involved in genome replication and packaging, and gene expression. Alternative splicing of the transcript from the single promoter controlling the cap gene produces three related structural proteins, VP1, VP2, and VP3, a total of 60 of which self-assemble to form the virus's icosahedral capsid in a ratio of approximately 1:1:10, respectively. VP1 is longest of the three VP proteins, and contains amino acids in its amino terminal region that are not present in VP2, which in turn is longer than VP3 and contains amino acids in its amino terminal region that are not present in VP3. The capsid protects the AAV genome, and also is responsible for binding specifically to receptors on the surface of target cells.

In addition to the rep and cap genes, intact AAV genomes have a relatively short (145 nucleotides in AAV2) sequence element positioned at each of their 5′ and 3′ ends called an inverted terminal repeat (ITR). ITRs contain nested palindromic sequences that can self-anneal through Watson-Crick base pairing to form a T-shaped, or hairpin secondary structure. In AAV2, ITRs have important functions required for the viral life cycle, including converting the single stranded DNA genome into double stranded form required for gene expression, as well as packaging by Rep proteins of single stranded AAV genomes into capsid assemblies.

After an AAV2 virion binds its cognate receptor on a cell surface, the viral particle enters the cell via endocytosis. Upon reaching the low pH of lysosomes, capsid proteins undergo a conformational change which allows the capsid to escape into the cytosol and then be transported into the nucleus. Once there, the capsid disassembles, releasing the genome which is acted on by cellular DNA polymerases to synthesize the second DNA strand starting at the ITR at the 3′ end, which functions as a primer after self-annealing. Expression of the rep and cap genes can then commence, followed by formation of new viral particles.

The relative simplicity of AAV structure and life cycle, and the fact that it is not known to be pathogenic in humans, inspired investigators to engineer AAV and convert it from a virus to a recombinant vector for gene therapy. Briefly, this was done by cloning the entire genome of AAV2, including both ITRs, into a plasmid, removing the rep and cap genes into a separate plasmid, and replacing them with a gene expression cassette comprising a heterologous transcription control region (promoter and optionally an enhancer) and gene of interest (which is sometimes referred to as a transgene). Thus, the only viral genome sequences retained in the vector genome are the ITRs due to their critical function in packaging and gene expression, without which AAV vectors could not be produced or function to express the gene of interest after transduction of target cells. Finally, to avoid the need for co-infection with a helper virus, genes for the so-called helper factors (such as, in the case of AdV, the E1A, E1B55K, E2A, E4orf6, and VA RNA helper factors) were cloned into a third plasmid. When the three plasmids are replicated to high number in bacteria, purified and transfected together into mammalian cells, such as HEK293 cells, Rep and VP proteins, and the AdV helper factors are expressed from their respective plasmids and function in the cells to assemble capsids, and package into them single stranded vector genomes replicated from the plasmids on which its sequence resides. Because the rep and cap genes exist in trans on a different plasmid, outside their usual context flanked by ITRs, they are not packaged into the vectors. Consequently, while vectors are able to bind to target cells and convey the expression cassette within their genomes into the cells, they cannot replicate and create new vector particles. For this reason, the term “transduction” is often used to refer to this process in place of the term “infection.” If the vector functions as intended, the expression cassette will be transcriptionally active and produce the gene product encoded by the gene of interest.

For use in connection with the methods of the disclosure, an AAV vector can include any gene of interest within an AAV vector genome of any sequence, structure, arrangement of functional sub-elements, and configuration known in the art to be suitable for its intended use, such as use in gene therapy. As AAV vectors are typically designed, choice of the gene of interest is limited only by the packaging capacity of the capsid, so that the gene's length when combined with all other elements in the genome required for vector function, such as the transcriptional regulatory region and the ITRs, does not exceed approximately 5 kilobases in the case of AAV2, although experimental strategies have been developed to surpass the packaging limit.

For purposes of gene therapy, the gene of interest can be any gene, the product of which would be understood to prevent or treat, but not necessarily cure, any disease or condition. In some embodiments, gene therapy is intended to prevent or treat a disease or condition characterized by an abnormally low amount or even absence of a product produced by a naturally occurring gene, such as might occur due to a loss of function mutation. Relating to such embodiments, the gene of interest can be one intended to compensate for the defective gene by providing the same or similar gene product when expressed. A non-limiting example would be a vector designed to express a functional version of clotting factor IX for use in gene therapy of hemophilia B, which is caused by a loss of function mutation in the native factor IX gene. In other embodiments, however, the gene of interest could be one intended to counteract the effects of a deleterious gain of function mutation in targeted cells. In some embodiments, the gene of interest can encode a transcriptional activator to increase the activity of an endogenous gene which produces a desirable gene product, or conversely a transcriptional repressor to decrease the activity of an endogenous gene which produces an undesirable gene product. In some embodiments, the gene of interest can encode for a protein, or an RNA molecule with a function distinct from encoding protein, such as a regulatory non-coding RNA molecule (e.g., micro RNA, small interfering RNA, piwi-acting RNA, enhancer RNA, or long non-coding RNA). Protein coding sequences in a gene of interest can be codon-optimized, and translation start sites (e.g., Kozak sequence) can be modified to increase or decrease their tendency to initiate translation. In some embodiments, the gene of interest can contain one or more open reading frames. In other embodiments, a vector genome can comprise more than one gene of interest, each part of its own separate transcriptional unit, or different products can be produced from a single transcriptional unit by inclusion of alternative splice sites.

Apart from the gene of interest, many other aspects of AAV vector genomes are amenable to design choice and optimization depending on the intended use of the vector. Without limitation, the transcriptional control region can be constitutively active, tissue specific, or inducible, and can include a promoter as well as one or more enhancer elements. A transcriptional control region can comprise the same nucleotide sequence as would occur in a gene naturally, or be modified to improve its function and/or reduce its length by changing, adding or removing nucleotides relative to a sequence found in nature, or even be entirely synthetic. In other embodiments, vector genomes can further comprise untranslated regions from the 5′ and/or 3′ end of genes, non-coding exons, introns, transcriptional termination signals (e.g., polyA signal sequence), elements that stabilize RNA transcripts, splice donor and acceptor sites, lox sites, binding sites for regulatory miRNAs, elements that enhance nuclear export of mRNAs, such as the woodchuck hepatitis virus post-transcriptional regulatory element (WPRE), and any other element demonstrated empirically to improve expression of the gene of interest, even if the mechanism may be uncertain.

In some embodiments, a vector genome can be designed for purposes of editing or otherwise modifying the genome of a target cell. For example, a vector genome can include a gene of interest flanked by homology arms intended to promote homologous recombination between the vector genome and the target cell genome. In another example, a vector genome can be designed to carry out CRISPR gene editing by expressing a guide RNA (gRNA) and/or an endonuclease, such as Cas9 or related endonucleases, such as SaCas9, capable of binding the gRNA and cleaving a DNA sequence targeted by the gRNA.

As known in the art, the ITRs typically used in AAV vectors originate from AAV2, but ITRs derived from other serotypes and naturally occurring AAV isolates, or hybrid, or even entirely synthetic ITRs, may be used as well. In some embodiments, vector genomes include two intact ITRs, one at each end of the single stranded DNA genome. In other embodiments, however, a third mutated ITR lacking a terminal resolution site can be positioned in the center of the genome, such as occurs in so-called self-complementary AAV (scAAV) genomes, which can self-anneal after capsid uncoating into double stranded form, permitting gene expression to proceed immediately without need for second strand synthesis, as is the case with conventional single stranded AAV genomes. ITRs from one type of AAV may be used in a genome that is contained in a capsid from the same type of AAV, or in a capsid from a different type of AAV, which are sometimes known as pseudotyped vectors. For example, AAV2 ITRs may be used in a genome that is encapsidated by an AAV2 capsid, or an AAV5 capsid (which is sometimes denoted AAV2/5) or another AAV capsid different from AAV2.

Just as there is wide latitude in the design of vector genomes, AAV vectors can be made using many different naturally occurring and modified AAV capsids. At one time, only six types of primate AAV had been isolated from biological samples (AAV1, AAV2, AAV3, AAV4, AAV5, and AAV6), the first five of which were sufficiently distinct structurally to be classified as different serotypes based on antibody cross reactivity experiments. Later, two novel AAVs, called AAV7 and AAV8 were discovered by PCR amplification of DNA from rhesus monkeys using primers targeting highly conserved regions in the cap genes of the previously discovered AAVs. Gao, G, et al., Novel adeno-associated viruses from rhesus monkeys as vectors for human gene therapy, PNAS (USA) 99(18):11854-11859 (2002). Subsequently, a similar approach was used to clone numerous novel AAVs from human and non-human primate tissues, vastly expanding the scope of known AAV cap protein sequences. Gao, G, et al., Clades of Adeno-Associated Viruses Are Widely Disseminated in Human Tissues, J Virol. 78(12):6381-6388 (2004). Many AAV cap protein sequences are highly similar to each other, or previously identified AAVs, and while often referred to as distinct AAV “serotypes,” not all such capsids would necessarily be expected to be immunologically distinguishable if tested by antibody cross reactivity.

Research has established that different AAV capsids have different tissue tropisms, as well as other properties that may make one capsid preferable over another for particular applications. For example, depending on which population is being tested, humans may have high neutralizing antibody titers as a result of exposure to naturally occurring AAVs, which can interfere with the ability of AAV vectors with the same or similar capsids to transduce target cells. Thus, in designing a vector for gene therapy, choice of capsid may in some cases be guided by the immunogenicity of the capsid, and/or the seroprevalence of the patients to be treated.

For use in connection with the methods of the disclosure, an AAV vector can include any capsid known in the art to be suitable for its intended use, such as use in gene therapy. Such capsids include those from naturally occurring AAVs, as well as modified or engineered capsids. For example, naturally occurring capsids can be modified by inserting peptides, or making amino acid substitutions, in the cap protein sequence intended to improve capsid function in some way, such as tissue tropism, immunogenicity, stability, or manufacturability. Other examples include novel capsids with improved properties created by swapping amino acids or domains from one known capsid to another (which are sometimes known as mosaic or chimeric capsids), or which are generated and selected employing DNA shuffling and directed evolution methods. In some embodiments, AAV vectors that can usefully be produced by host cells and purified with the methods of the disclosure include those that use any of the following capsids: AAV1, AAV2, AAV3, AAV3b, AAV4, AAV5, AAV6, AAV7, AAV8, AAV9, Rh10, Rh74, AAV-DJ, AAV-PH P.B, An c80, AAV2.5, and AAV2i8, with many others being possible.

As noted above, AAV infection begins when a virion's capsid binds specifically to a receptor on the surface of a target cell. The virion is then taken into the cell via endocytosis, and trafficked to the nucleus, where the capsid uncoats to reveal the genome. Different capsids have been shown to bind specifically to different cellular receptors, which may explain, at least in part, the different tissue tropisms that have been observed among different AAV serotypes and variants. Initial attachment by AAV to cells in many cases appears to involve binding by capsids to glycan moieties displayed on cell surface proteins, with other cell surface proteins playing an important role as co-receptors involved with viral entry. For example, AAV1, AAV5, and AAV6 have been shown to bind to N-linked sialic acid, AAV4 to O-linked sialic acid, and AAV9 to terminal N-linked galactose. Other capsids have been shown to bind specifically to heparan-sulfate proteoglycan (HSPG), including AAV2, AAV3, AAV3b, AAV6, AAV13, and AAV-DJ. The cell surface protein known as AAVR is apparently required for entry by a number of AAV serotypes, but other proteins, such as certain integrins and laminin receptor may also be involved depending on the capsid serotype. See, e.g., Huang, LY, Parvovirus Glycan Interactions, Curr. Opin. Virol. (2014) 7:108-118; Zhang, R, et al., Divergent engagements between adeno-associated viruses with their cellular receptor AAVR, Nat. Comms. (2019) 10:3760; Havlik, L P, Receptor Switching in Newly Evolved Adeno-associated Viruses, J. Virol. (2021) 95(19):e00587-21.

In some embodiments, AAV vectors comprising any known or as yet uncharacterized capsid can be purified with the methods of the disclosure, whereas in other embodiments, AAV vectors that can be purified with the methods of the disclosure comprise a capsid that binds more strongly to sialic acid or galactose as compared to HSPG, or does not specifically bind, or binds only weakly to HSPG. Thus, some embodiments, AAV vectors comprising capsids from AAV1, AAV2, AAV3, AAV3b, AAV4, AAV5, AAV6, AAV9, AAV13, and AAV-DJ can be purified with the methods of the disclosure, whereas in other embodiments, AAV vectors comprising capsids from AAV1, AAV4, AAV5, and AAV9 can be purified with the methods of the disclosure. Specific binding affinity or avidity of a capsid to receptors of any kind can be determined using any technique familiar to those of ordinary skill in the art, such as surface plasmon resonance, or other methods. In some embodiments, AAV vectors that can be purified with the methods of the disclosure comprise a capsid that binds more strongly to sialic acid or galactose as compared to HSPG by a factor of at least or about 2, 3, 4, 5, 6, 7, 8, 9, 10, 20, 50, 100, 250, 500, 1000 times or more, or some other factor.

As known in the art, viral vectors can be produced, including at large scale, in a number of ways. AAV vectors, for example, can be made in mammalian or insect cells and then purified. The traditional approach that does not rely on coinfection with a helper virus involves use of three plasmids, as discussed above. One plasmid contains genes for helper virus factors, a second contains the AAV genome sequence in double stranded form, and the third contains AAV rep and cap genes. The rep/cap plasmid often contains a rep gene from AAV2, although this is not a requirement, and the cap gene sequence is chosen based on which AAV cap protein is desired to constitute the capsid. In practice, the three plasmids are often separately replicated in bacteria, purified, mixed in solution together in predetermined proportions, and then mixed with a transfection agent. The transfection mixture is then used to transfect suitable mammalian host cells (in adherent or suspension cell culture) which are incubated for sufficient time (e.g., 48 to 72 hours, etc.) and under conditions sufficient for the host cells to express the helper factors and the rep and cap genes, and for AAV vector genome to be replicated from its plasmid template and packaged into capsids. In some embodiments, the host cells are HEK293 cells, which constitutively express AdV helper factors E1A and E1B, such that the helper plasmid only need contain the AdV E2A, E4ORF6, and VA RNA genes. Use of other mammalian host cells that do not produce AdV or other viral helper factor on their own would necessitate use of a helper plasmid containing whichever helper factors are missing or are otherwise required. Although the so-called triple transfection method described above is commonly employed, there is no requirement that the helper factor, and rep and cap genes, be provided on separate plasmids. In principle all these genes could be housed in one plasmid, for example, in which case two plasmids can be used in the transfection.

Seeking more efficient methods of producing AAV vector at large scale, stable cell lines have been created that contain some but not all the components that would otherwise need to be introduced into cells by transient transfection. Packaging cell lines contain stably integrated AAV rep and cap genes. Production of AAV in packaging cells requires them to be transiently transfected with a plasmid containing an AAV vector genome and infected with a helper virus. It is also possible to produce AAV vectors in packaging cells without transfection by first infecting them with an AdV (either wild type or in which the E2b gene is deleted) which supplies AdV E1 gene products, which induce rep and cap expression in the cells, as well as helper factors required for AAV replication, followed by infection with a replication deficient hybrid AdV in which an AAV vector genome replaces the E1 gene in the genome of the hybrid virus. Producer cell lines contain stably integrated AAV rep and cap genes, and also an AAV vector genome. Production of AAV in producer cells requires them to be infected with a helper virus. Packaging and producer cells are described further in, e.g., Martin, J, et al., Generation and characterization of adeno-associated virus producer cell lines for research and preclinical vector production, Hum. Gene Methods, 24:253-269 (2013); Gao, G P, et al., High-titer adeno-associated viral vectors from a Rep/Cap cell line and hybrid shuttle virus, Hum. Gene Ther., 9:2353-62 (1998); Martin, J, et al., Generation and Characterization of Adeno-Associated Virus Producer Cell Lines for Research and Preclinical Vector Production, Hum. Gene Ther. Meth., 24:253-69 (2013); Clement, N and J C Grieger, Manufacturing of recombinant adeno-associated viral vectors for clinical trials, Mol. Ther. Meth. & Clin. Dev. (2016) 3, 16002 (doi:10.1038/mtm.2016.2). Other cellular systems for producing AAV vectors in mammalian cells, including at commercial scale, are possible.

The baculovirus system has also been employed to produce AAV vector. In this system, Sf9 insect cells are infected with recombinant baculovirus vectors that variously contain the AAV rep and cap genes and the AAV genome. The exogenous genes are expressed, followed by genome packaging into vector particles within the cells. In early versions of the system, each component, rep, cap, and genome, were carried by three separate baculoviruses. Later, modifications were made, such as combining rep and cap into a single baculovirus, so that only two types of baculovirus were required, as well as producing Sf9 cell lines containing stably integrated AAV rep and cap genes, which only require infection with a single type of recombinant baculovirus containing an AAV vector genome. Use of the baculovirus system to produce AAV vector is described further in, e.g., Urabe, M, et al., Insect Cells as a Factory to Produce Adeno-Associated Virus Type 2 Vectors, Hum. Gene Ther., 13:1935-43 (2002); Virag, T, et al., Producing recombinant adeno-associated virus in foster cells: Overcoming production limitations using a baculovirus-insect cell expression strategy, Hum. Gene Ther., 20:807-17 (2009); Smith, R H, et al., A simplified baculovirus-AAV expression vector system coupled with one-step affinity purification yields high-titer rAAV stocks from insect cells, Mol. Ther., 17:1888-96 (2009); Mietzsch, M, et al., OneBac: platform for scalable and high-titer production of adeno-associated virus serotype 1-12 vectors for gene therapy, Hum. Gene. Ther. 25(3):212-22 (2014). Other cellular systems for producing AAV vectors in insect cells, including at commercial scale, are possible.

Lysing Host Cells

Host cells comprising a desired biological product can be lysed in any way known in the art to be effective to disrupt a cell's plasma membrane, permitting the cell's internal contents to make contact with the surrounding medium, while not denaturing the biological product sought to be purified. Lysing host cells produces a crude host cell lysate comprising host cell DNA and biological product, among other cellular components, dispersed in the fluid in which the cells had been suspended, or which had otherwise surrounded the cells immediately before lysis.

In some embodiments of the disclosure, host cell lysis can be effected mechanically, such as with a high pressure homogenizer or bead mill, or non-mechanically, which can encompass physical, chemical, or biological methods. Examples of physical methods include exposing cells to heating, freeze-thaw cycles, osmotic shock, sonication or cavitation; examples of chemical methods include treating cells with alkali or detergents; and examples of biological methods include treating cells with enzymes. More information about lysis methods can be found in Islam, M S, et al., A Review on Macroscale and Microscale Cell Lysis Methods, Micromachines 8, 83 (pp. 1-27) (2017).

In some embodiments, host cell lysis is effected by contacting the host cells with a detergent in sufficient concentration to cause disruption, dissolution, or lysis of the cells' plasma membrane. As noted above, host cells may be grown and maintained in adherent cell culture or suspension cell culture. In some embodiments, lysis of host cells grown in adherent cell culture can conveniently be performed in several ways. The growth medium can be removed and then a lysis solution comprising a detergent, and optionally other components such as buffers, at a final concentration sufficient to cause cell lysis can be added to the container in which the cells are grown and then be caused to make contact with the cells until cell lysis results. The container may be agitated, rocked, etc. to cause effective distribution of the lysis solution over the cells and mixing of the lysed cells and their contents with the lysis solution. Alternatively, a concentrated stock solution comprising the detergent, and optionally other components such as buffers, can be prepared and added directly to the growth media (or other physiologically compatible fluid in which the cells are being maintained, such as phosphate buffered saline (PBS), or the like) to a desired final detergent concentration (e.g., as a % weight by volume, % weight by weight, or molarity) sufficient to cause cell lysis. The media and lysis solution can then be mixed and caused to make contact with the adherent cells in the container until cell lysis results. In other embodiments, host cells grown in adherent cell culture can be chemically or enzymatically detached from their substrate, after which a lysis solution, as described above, is added to the container and allowed to make contact with cells in suspension until cell lysis results. Alternatively, the detached cells can be removed from their growth container and transferred in suspension to a new container where lysis would be performed, as described above.

In some embodiments, host cells grown or maintained in suspension cell culture can be lysed by adding to the growth media (or other physiologically compatible fluid in which the cells are maintained, such as phosphate buffered saline (PBS), or the like) a concentrated stock solution comprising the detergent, and optionally other components such as buffers, to a desired final detergent concentration (e.g., as a % weight by volume, % weight by weight, or molarity) sufficient to cause cell lysis. The media and lysis solution can then be mixed to evenly distribute the detergent throughout the culture volume, and allowed to contact the cells until cell lysis results. In some embodiments, the host cells are grown or maintained in bioreactors of any desired volume to which the detergent lysis solution is added as one or more boluses, or continually until the entire desired volume of lysis solution has been added. A detergent lysis solution can be added to a bioreactor, or any container in which host cells are to be lysed, in any way that is known in the art, for example, from above, such as through a tube positioned above the fluid in which the host cells are suspended, or from below the surface of such fluid at any desired level of the bioreactor, such as through subsurface addition lines or tubes. Mixing of the media in which the cells are suspended and the lysis solution can proceed over the entire period during which cells are lysed, or for a shorter period followed by an incubation period in which cells are allowed to lyse without mixing or agitation. Mixing can be performed in any way that is known the art, such as using impellers or pumps. In some embodiments, the host cells and the media in which they were grown in suspension culture can be separated, such as by allowing host cells to settle out, and the media removed and replaced with a different fluid, such as fresh media of the same or different kind, or some other physiologically compatible fluid, to which the detergent lysis solution is then added followed by mixing and cell lysis. This can occur in the same container in which the cells were grown or in a new container of any suitable size.

Any detergent known in the art to be effective for causing host cell lysis while not damaging or denaturing the desired biological product can be used, although use of otherwise denaturing detergents may be possible if their concentration is low, or if cells are exposed to them for limited periods of time. Detergents for use in the methods of the disclosure can be anionic detergents, cationic detergents, zwitterionic detergents or non-ionic detergents. Examples of anionic detergents include sodium deoxycholate and alkyl sulfates, such as sodium dodecyl sulfate, with others being possible. Many examples of cationic detergents are known in the art. Examples of zwitterionic detergents include (3-((3-cholamidopropyl) dimethylammonio)-1-propanesulfonate) (CHAPS) and 3-([3-cholamidopropyl] dimethylammonio)-2-hydroxy-1-propanesulfonate (CHAPSO), with others being possible. Examples of non-ionic detergents include n-alkyl-beta-D-maltopyranosides (such as where the alkyl group is octyl, nonyl, decyl, undecyl, or dodecyl), n-alkyl-beta-D-glucopyranosides (such as where the alkyl group is octyl, nonyl, decyl, undecyl, or dodecyl), n-alkyl-beta-D-thioglucopyranosides (such as where the alkyl group is octyl, nonyl, decyl, undecyl, or dodecyl), digitonin, polyethylene glycol sorbitan monolaurate (e.g., polysorbate 20 (Tween 20), polysorbate 80 (Tween 80)), Brij 35, Brij 58, and alkylphenol ethoxylate detergents.

In some embodiments, non-ionic detergents useful in the methods of the disclosure for lysing host cells are alkylphenol ethoxylate detergents, such as 2-alkylphenol ethoxylate, 3-alkylphenol ethoxylate, or 4-alkylphenol ethoxylate detergents, which are provided by the general formula CₙH₂ₙ₊₁-Phenyl-O—[—CH2-CH2-O—]ₓ—H, where the alkyl group can be linear or branched, n is 1, 2, 3, 4, 5, 6, 7, 8, 9, 10, 11, 12, 13, 14, 15, 16, 17, 18, 19, 20, or some other integer, and x is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or some other integer.

In some embodiments, nonionic detergents useful in the methods of the disclosure for lysing host cells are octylphenol ethoxylate detergents, such as 2-octylphenol ethoxylate, 3-octylphenol ethoxylate, or 4-octylphenol ethoxylate detergents, which are provided by the general formula C₈H₁₇-Phenyl-O—[—CH2-CH2-O—]ₓ—H, where the octyl group can be linear or branched, and x is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or some other integer.

In some embodiments, nonionic detergents useful in the methods of the disclosure for lysing host cells are 4-octylphenol ethoxylate detergents having the following structure:

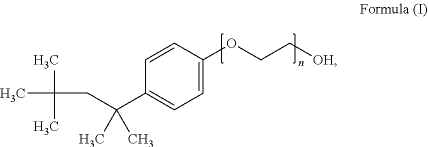

where n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or some other integer.

Exemplary octylphenol ethoxylate detergents include those in the Triton X series, such as Triton X-15, X-35, X-45, X-100, X-165, X-305, X-405, X-102, X-114, or X-705. Exemplary octylphenol ethoxylate detergents also include those in the Igepal series, such as Igepal CA-630 and CA-720. In some embodiments, octylphenol ethoxylate detergents for use in the methods of the disclosure are known by the following (I) chemical names: 4-(1,1,3,3-tetramethylbutyl)phenyl-polyethylene glycol; polyethylene glycol 4-tert-octylphenyl ether; t-octylphenoxypolyethoxyethanol; polyethylene glycol tert-octylphenyl ether; polyoxyethylene (10) isooctylcyclohexyl ether; (1,1,3,3-tetramethylbutyl)phenyl-polyethylene glycol; 4-(1,1,3,3-tetramethylbutyl)phenyl-polyethylene glycol; polyoxyethylene (40) isooctylphenyl ether; polyethylene glycol tert-octylphenyl ether; polyoxyethylene (40) isooctylcyclohexyl ether; polyoxyethylene (12) isooctylphenyl ether; polyoxyethylene (12) octylphenyl ether, branched; (II) chemical formulas: t-oct-C₆H₄—(OCH₂CH₂)ₓOH, x=^{˜}5; 4-(C₈H₁₇)C₆H₁₀(OCH₂CH₂)ₙOH, n^{˜}10; t-Oct-C₆H₄—(OCH₂CH₂)ₓOH, x=9-10; (C₂H₄O)n C₁₄H₂₂O, n=7 or 8; or (III) CAS Registry Numbers: 9036-19-5, 92046-34-9, and 9002-93-1.

In some embodiments, nonionic detergents useful in the methods of the disclosure for lysing host cells are nonylphenol ethoxylate detergents, such as 2-nonylphenol ethoxylate, 3-nonylphenol ethoxylate, or 4-nonylphenol ethoxylate detergents, which are provided by the general formula C₉H₁₉-Phenyl-O—[—CH2-CH2-O—]ₓ—H, where the nonyl group can be linear or branched, and x is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or some other integer.

Exemplary nonylphenol ethoxylate detergents include those in the Tergitol NP series, including NP-4, NP-6, NP-7, NP-8, NP-9, NP-9.5, NP-10, NP-11, NP-12, NP-13, NP-15, NP-30, NP-40, and NP-50. Exemplary nonylphenol ethoxylate detergents also include those in the Igepal series, such as Igepal CO-520, CO-630, CO-720, and CO-890. In some embodiments, nonylphenol ethoxylate detergents for use in the methods of the disclosure are known by the following (I) chemical names: polyoxyethylene (5) nonylphenylether, branched; polyoxyethylene (9) nonylphenylether, branched; polyoxyethylene (12) nonylphenyl ether, branched; polyoxyethylene (40) nonylphenyl ether, branched; (II) chemical formulas: (C₂H₄O)ₙ·C₁₅H₂₄O, n^{˜}5; (C₂H₄O)ₙ·C₁₅H₂₄O·n=9-10; (C₂H₄O)ₙ·C₁₅H₂₄O, n=10.5-12; (C₂H₄O)ₙ·C₁₅H₂₄O n=40; or (III) CAS Registry Numbers: 68412-54-4.

In some embodiments, alkylphenol ethoxylate detergent compositions, such as octylphenol ethoxylate and nonylphenol ethoxylate detergent compositions, comprise a heterogenous population of species having different alkyl chain structures, e.g., some being linear and some being branched of different configurations, as well as hydrophile chains with different numbers of ethoxylate (OCH 2 CH 2) monomers. In some embodiments, alkylphenol ethoxylate detergent compositions, such as octylphenol ethoxylate and nonylphenol ethoxylate detergent compositions, can be characterized by the average number of ethoxylate monomers per molecule, for example, an average of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 ethoxylate monomers per molecule, or between 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, or 19-20 ethoxylate monomers per molecule.

In some embodiments, detergent compositions for use in the methods of the disclosure comprise octylphenol ethoxylate detergents having the structure of Formula (I), where the average value of n is between 9 and 10.

In some embodiments, particularly when host cells are grown or maintained in suspension culture, the step of host cell lysis can be performed at a predetermined viable cell density, meaning the number of viable host cells in a defined volume of media, or other physiologically compatible fluid in which they are suspended, for example viable cells per milliliter (vc/mL). Cell viability can be determined using any technique known in the art. For example, a sample of cells can be withdrawn from the culture in which they are grown or maintained, mixed with a vital dye such as trypan blue, and then the total number of cells excluding the dye counted on a hemocytometer from which the number of viable cells per mL (or any other volume) can readily be calculated. Alternatively, viable cell density can be monitored in real time using sensors, such as permittivity sensors, more information about which can be found, e.g., in Metze, S, et al., Monitoring online biomass with a capacitance sensor during scale-up of industrially relevant CHO cell culture fed-batch processes in single-use bioreactors, Bioprocess Biosys. Eng. 43:193-205 (2020).

In some embodiments, host cells producing a desired biological product, such as an AAV vector, are lysed (harvested) at a certain viable cell density, where such viable cell density can be at least or about 0.01×10⁶ vc/mL, 0.1×10⁶ vc/mL, 1×10⁶ vc/mL, 2×10⁶ vc/mL, 3×10⁶ vc/mL, 4×10⁶ vc/mL, 5×10⁶ vc/mL, 6×10⁶ vc/mL, 7×10⁶ vc/mL, 8×10⁶ vc/mL, 9×10⁶ vc/mL, 10×10⁶ vc/mL, 11×10⁶ vc/mL, 12×10⁶ vc/mL, 13×10⁶ vc/mL, 14×10⁶ vc/mL, 15×10⁶ vc/mL, 16×10⁶ vc/mL, 17×10⁶ vc/mL, 18×10⁶ vc/mL, 19×10⁶ vc/mL, 20×10⁶ vc/mL, 21×10⁶ vc/mL, 22×10⁶ vc/mL, 23×10⁶ vc/mL, 24×10⁶ vc/mL, 25×10⁶ vc/mL, 26×10⁶ vc/mL, 27×10⁶ vc/mL, 28×10⁶ vc/mL, 29×10⁶ vc/mL, 30×10⁶ vc/mL, or higher, or a range of viable cell density including and between any two of the foregoing values, such as about 2×10⁶ to 25×10⁶ vc/mL; 5×10⁶ to 25×10⁶ vc/mL; 2×10⁶ to 30×10⁶ vc/mL; 5×10⁶ to 30×10⁶ vc/mL; 10×10⁶ to 20×10⁶ vc/mL; 11×10⁶ to 20×10⁶ vc/mL; 12×10⁶ to 20×10⁶ vc/mL; 13×10⁶ to 20×10⁶ vc/mL; 14×10⁶ to 20×10⁶ vc/mL; 15×10⁶ to 20×10⁶ vc/mL; 16×10⁶ to 20×10⁶ vc/mL; 17×10⁶ to 20×10⁶ vc/mL; 18×10⁶ to 20×10⁶ vc/mL; 19×10⁶ to 20×10⁶ vc/mL; 10×10⁶ to 21×10⁶ vc/mL; 11×10⁶ to 21×10⁶ vc/mL; 12×10⁶ to 21×10⁶ vc/mL; 13×10⁶ to 21×10⁶ vc/mL; 14×10⁶ to 21×10⁶ vc/mL; 15×10⁶ to 21×10⁶ vc/mL; 16×10⁶ to 21×10⁶ vc/mL; 17×10⁶ to 21×10⁶ vc/mL; 18×10⁶ to 21×10⁶ vc/mL; 19×10⁶ to 21×10⁶ vc/mL; 20×10⁶ to 21×10⁶ vc/mL; 10×10⁶ to 22×10⁶ vc/mL; 11×10⁶ to 22×10⁶ vc/mL; 12×10⁶ to 22×10⁶ vc/mL; 13×10⁶ to 22×10⁶ vc/mL; 14×10⁶ to 22×10⁶ vc/mL; 15×10⁶ to 22×10⁶ vc/mL; 16×10⁶ to 22×10⁶ vc/mL; 17×10⁶ to 22×10⁶ vc/mL; 18×10⁶ to 22×10⁶ vc/mL; 19×10⁶ to 22×10⁶ vc/mL; 20×10⁶ to 22×10⁶ vc/mL; 21×10⁶ to 22×10⁶ vc/mL; 10×10⁶ to 23×10⁶ vc/mL; 11×10⁶ to 23×10⁶ vc/mL; 12×10⁶ to 23×10⁶ vc/mL; 13×10⁶ to 23×10⁶ vc/mL; 14×10⁶ to 23×10⁶ vc/mL; 15×10⁶ to 23×10⁶ vc/mL; 16×10⁶ to 23×10⁶ vc/mL; 17×10⁶ to 23×10⁶ vc/mL; 18×10⁶ to 23×10⁶ vc/mL; 19×10⁶ to 23×10⁶ vc/mL; 20×10⁶ to 23×10⁶ vc/mL; 21×10⁶ to 23×10⁶ vc/mL; 10×10⁶ to 24×10⁶ vc/mL; 11×10⁶ to 24×10⁶ vc/mL; 12×10⁶ to 24×10⁶ vc/mL; 13×10⁶ to 24×10⁶ vc/mL; 14×10⁶ to 24×10⁶ vc/mL; 15×10⁶ to 24×10⁶ vc/mL; 16×10⁶ to 24×10⁶ vc/mL; 17×10⁶ to 24×10⁶ vc/mL; 18×10⁶ to 24×10⁶ vc/mL; 19×10⁶ to 24×10⁶ vc/mL; 20×10⁶ to 24×10⁶ vc/mL; 21×10⁶ to 24×10⁶ vc/mL; 22×10⁶ to 24×10⁶ vc/mL; 23×10⁶ to 24×10⁶ vc/mL; 10×10⁶ to 25×10⁶ vc/mL; 11×10⁶ to 25×10⁶ vc/mL; 12×10⁶ to 25×10⁶ vc/mL; 13×10⁶ to 25×10⁶ vc/mL; 14×10⁶ to 25×10⁶ vc/mL; 15×10⁶ to 25×10⁶ vc/mL; 16×10⁶ to 25×10⁶ vc/mL; 17×10⁶ to 25×10⁶ vc/mL; 18×10⁶ to 25×10⁶ vc/mL; 19×10⁶ to 25×10⁶ vc/mL; 20×10⁶ to 25×10⁶ vc/mL; 21×10⁶ to 25×10⁶ vc/mL; 22×10⁶ to 25×10⁶ vc/mL; 23×10⁶ to 25×10⁶ vc/mL; 24×10⁶ to 25×10⁶ vc/mL; 10×10⁶ to 26×10⁶ vc/mL; 11×10⁶ to 26×10⁶ vc/mL; 12×10⁶ to 26×10⁶ vc/mL; 13×10⁶ to 26×10⁶ vc/mL; 14×10⁶ to 26×10⁶ vc/mL; 15×10⁶ to 26×10⁶ vc/mL; 16×10⁶ to 26×10⁶ vc/mL; 17×10⁶ to 26×10⁶ vc/mL; 18×10⁶ to 26×10⁶ vc/mL; 19×10⁶ to 26×10⁶ vc/mL; 20×10⁶ to 26×10⁶ vc/mL; 21×10⁶ to 26×10⁶ vc/mL; 22×10⁶ to 26×10⁶ vc/mL; 23×10⁶ to 26×10⁶ vc/mL; 24×10⁶ to 26×10⁶ vc/mL; 25×10⁶ to 26×10⁶ vc/mL; 10×10⁶ to 27×10⁶ vc/mL; 11×10⁶ to 27×10⁶ vc/mL; 12×10⁶ to 27×10⁶ vc/mL; 13×10⁶ to 27×10⁶ vc/mL; 14×10⁶ to 27×10⁶ vc/mL; 15×10⁶ to 27×10⁶ vc/mL; 16×10⁶ to 27×10⁶ vc/mL; 17×10⁶ to 27×10⁶ vc/mL; 18×10⁶ to 27×10⁶ vc/mL; 19×10⁶ to 27×10⁶ vc/mL; 20×10⁶ to 27×10⁶ vc/mL; 21×10⁶ to 27×10⁶ vc/mL; 22×10⁶ to 27×10⁶ vc/mL; 23×10⁶ to 27×10⁶ vc/mL; 24×10⁶ to 27×10⁶ vc/mL; 25×10⁶ to 27×10⁶ vc/mL; 26×10⁶ to 27×10⁶ vc/mL; 10×10⁶ to 28×10⁶ vc/mL; 11×10⁶ to 28×10⁶ vc/mL; 12×10⁶ to 28×10⁶ vc/mL; 13×10⁶ to 28×10⁶ vc/mL; 14×10⁶ to 28×10⁶ vc/mL; 15×10⁶ to 28×10⁶ vc/mL; 16×10⁶ to 28×10⁶ vc/mL; 17×10⁶ to 28×10⁶ vc/mL; 18×10⁶ to 28×10⁶ vc/mL; 19×10⁶ to 28×10⁶ vc/mL; 20×10⁶ to 28×10⁶ vc/mL; 21×10⁶ to 28×10⁶ vc/mL; 22×10⁶ to 28×10⁶ vc/mL; 23×10⁶ to 28×10⁶ vc/mL; 24×10⁶ to 28×10⁶ vc/mL; 25×10⁶ to 28×10⁶ vc/mL; 26×10⁶ to 28×10⁶ vc/mL; 27×10⁶ to 28×10⁶ vc/mL; 10×10⁶ to 29×10⁶ vc/mL; 11×10⁶ to 29×10⁶ vc/mL; 12×10⁶ to 29×10⁶ vc/mL; 13×10⁶ to 29×10⁶ vc/mL; 14×10⁶ to 29×10⁶ vc/mL; 15×10⁶ to 29×10⁶ vc/mL; 16×10⁶ to 29×10⁶ vc/mL; 17×10⁶ to 29×10⁶ vc/mL; 18×10⁶ to 29×10⁶ vc/mL; 19×10⁶ to 29×10⁶ vc/mL; 20×10⁶ to 29×10⁶ vc/mL; 21×10⁶ to 29×10⁶ vc/mL; 22×10⁶ to 29×10⁶ vc/mL; 23×10⁶ to 29×10⁶ vc/mL; 24×10⁶ to 29×10⁶ vc/mL; 25×10⁶ to 29×10⁶ vc/mL; 26×10⁶ to 29×10⁶ vc/mL; 27×10⁶ to 29×10⁶ vc/mL; 28×10⁶ to 29×10⁶ vc/mL; 10×10⁶ to 30×10⁶ vc/mL; 11×10⁶ to 30×10⁶ vc/mL; 12×10⁶ to 30×10⁶ vc/mL; 13×10⁶ to 30×10⁶ vc/mL; 14×10⁶ to 30×10⁶ vc/mL; 15×10⁶ to 30×10⁶ vc/mL; 16×10⁶ to 30×10⁶ vc/mL; 17×10⁶ to 30×10⁶ vc/mL; 18×10⁶ to 30×10⁶ vc/mL; 19×10⁶ to 30×10⁶ vc/mL; 20×10⁶ to 30×10⁶ vc/mL; 21×10⁶ to 30×10⁶ vc/mL; 22×10⁶ to 30×10⁶ vc/mL; 23×10⁶ to 30×10⁶ vc/mL; 24×10⁶ to 30×10⁶ vc/mL; 25×10⁶ to 30×10⁶ vc/mL; 26×10⁶ to 30×10⁶ vc/mL; 27×10⁶ to 30×10⁶ vc/mL; 28×10⁶ to 30×10⁶ vc/mL; or 29×10⁶ to 30×10⁶ vc/mL, or some other range. In some embodiments, the host cells are HEK293 cells in suspension culture.

In some embodiments, host cells producing a desired biological product, such as an AAV vector, are lysed while suspended in a volume of a physiologically compatible fluid, such as the media in which they were grown, or in some embodiments the media in which they were transfected (which can be the same media as that in which they were grown), where such volume can be at least or about 2 liters (L), 5 L, 10 L, 20 L, 25 L, 50 L, 75 L, 100 L, 150 L, 200 L, 300 L, 400 L, 500 L, 600 L, 700 L, 750 L, 800 L, 900 L, 1000 L, 1250 L, 1500 L, 1750 L, 2000 L, 3000 L, 4000 L, 5000 L, or a greater volume, or a range including and between any of the foregoing volumes, such as 2 L to 5 L, 2 L to 10 L, 2 L to 50 L, 2 L to 100 L, 5 L to 10 L, 5 L to 50 L, 5 L to 100 L, 10 L to 50 L, 10 L to 100 L, 50 L to 250 L, 50 L to 500 L, 100 L to 250 L, 100 L to 500 L, 100 L to 1000 L, 250 L to 500 L, 250 L to 1000 L, 250 L to 2000 L, 500 L to 1000 L, 500 L to 2000 L, 500 L to 5000 L, or some other range. In some embodiments, the volume is that of a sample of the host cells, which sample can comprise the entire volume of a suspension cell culture in a bioreactor, for example. In some embodiments, the host cells are HEK293 cells in suspension culture. In other embodiments, before lysis, host cells can be concentrated into a volume of physiologically compatible fluid that is smaller compared to the volume of media in which they were grown.

The final concentration of detergent added to a suspension of host cells or mixture thereof and detergent solution (or in a cell lysis solution, in the case of where adherent cells are lysed while attached to a substrate) can be any concentration effective to lyse the host cells while not damaging or denaturing a desired biological product, such as an AAV vector, released from the cells after lysis. Concentrations can be expressed in terms of percentage or molarity, and if expressed as a percentage, can be calculated as % weight by volume or % weight by weight, the values of which will not be significantly different for dilute aqueous solutions.

In some embodiments, the final concentration of detergent (for example, an anionic detergent, cationic detergent, zwitterionic detergent or non-ionic detergent) can be at least or about 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, 1.10%, 1.20%, 1.25%, 1.30%, 1.40%, 1.50%, 1.60%, 1.70%, 1.80%, 1.90%, 2.00%, 3.00%, 4.00%, or 5.00%, or a range of concentrations including and between any two of the foregoing values, such as from about 0.05% to 5.00%, 0.05% to 1.25%, 0.10% to 2.50%, 0.10% to 0.90%, 0.20% to 0.80%, 0.20% to 1.25%, 0.20% to 0.75%, 0.25% to 0.75%, 0.25% to 0.65%, 0.20% to 0.70%, 0.30% to 0.70%, 0.35% to 0.65%, 0.40% to 0.60%, 0.45% to 0.55%, or some other range. In some embodiments, the detergent used to lyse host cells is a non-ionic detergent, for example an alkylphenol ethoxylate detergent, such as an octylphenol ethoxylate detergent (e.g., a 4-octylphenol ethoxylate detergent) or a nonylphenol ethoxylate detergent (e.g., a 4-nonylphenol ethoxylate detergent), with a specific non-limiting example being Triton X-100, any of which can be used to lyse host cells at a final concentration of at least or about 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, 1.10%, 1.20%, 1.25%, 1.30%, 1.40%, 1.50%, 1.60%, 1.70%, 1.80%, 1.90%, 2.00%, 3.00%, 4.00%, or 5.00%, or a range of concentrations including and between any two of the foregoing values, such as from about 0.05% to 5.00%, 0.05% to 1.25%, 0.10% to 2.50%, 0.10% to 0.90%, 0.20% to 0.80%, 0.20% to 1.25%, 0.20% to 0.75%, 0.25% to 0.75%, 0.25% to 0.65%, 0.20% to 0.70%, 0.30% to 0.70%, 0.30% to 0.60%, 0.35% to 0.65%, 0.40% to 0.60%, 0.45% to 0.55%, or some other range.

In some embodiments, a sample of host cells in suspension is lysed by adding to the suspension a solution comprising Triton X-100, wherein the final concentration of Triton X-100 in the mixture is about 0.30% to 0.70%, 0.35% to 0.65%, 0.40% to 0.60%, 0.45% to 0.55%, or about 0.5%. In some embodiments, the host cells are HEK293 cells in suspension culture. In some embodiments, the host cells produce AAV vector.

In some embodiments, Triton X-100 at a final concentration of about 0.5% is effective to lyse host cells even at high viable cell densities at the time of lysis (harvest), such as at least or about 9×10⁶ vc/mL, 10×10⁶ vc/mL, 11×10⁶ vc/mL, 12×10⁶ vc/mL, 13×10⁶ vc/mL, 14×10⁶ vc/mL, 15×10⁶ vc/mL, 16×10⁶ vc/mL, 17×10⁶ vc/mL, 18×10⁶ vc/mL, 19×10⁶ vc/mL, 20×10⁶ vc/mL, 21×10⁶ vc/mL, 22×10⁶ vc/mL, 23×10⁶ vc/mL, 24×10⁶ vc/mL, 25×10⁶ vc/mL, 26×10⁶ vc/mL, 27×10⁶ vc/mL, 28×10⁶ vc/mL, 29×10⁶ vc/mL, 30×10⁶ vc/mL, or a range of viable cell density including and between any two of the foregoing values, such as from about 9×10⁶ vc/mL to 15×10⁶ vc/mL, 10×10⁶ vc/mL to 14×10⁶ vc/mL, 18×10⁶ vc/mL to 24×10⁶ vc/mL, 10×10⁶ vc/mL to 30×10⁶ vc/mL, or 15×10⁶ vc/mL to 25×10⁶ vc/mL. In the latter two exemplary ranges, a final concentration of 0.5% Triton X-100 is equivalent to 0.05% to 0.017% per 10⁶ viable host cells per mL, or to 0.033% to 0.02% per 10⁶ viable host cells per mL, respectively. These factors can be used to calculate final concentration ranges of Triton X-100 that could be used to effectively lyse host cells at any particular viable cell density. For example, in some embodiments, Triton X-100 at a final concentration of about 0.17% to 0.5%, or about 0.2% to 0.33%, is effective to lyse host cells at a viable cell density of about 10×10⁶ vc/mL, Triton X-100 at a final concentration of about 0.33% to 1.0%, or about 0.4% to 0.67%, is effective to lyse host cells at a viable cell density of about 20×10⁶ vc/mL, and Triton X-100 at a final concentration of about 0.5% to 1.5%, or about 0.6% to 1%, is effective lyse host cells at a viable cell density of about 30×10⁶ vc/mL. In like fashion, in some embodiments, Triton X-100 at a final concentration of about 1% to 3%, or about 1.2% to 2%, is effective lyse host cells at a viable cell density of about 60×10⁶ vc/mL. In some non-limiting embodiments, the host cells are HEK293 cells grown in suspension culture which produce an AAV vector.

In some other embodiments, Triton X-100 added to a sample of host cells, such as HEK293 cells grown in suspension culture, at a final concentration of about 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, 0.020% to 0.060% per 1×10⁶ vc/mL, 0.023% to 0.070% per 1×10⁶ vc/mL, 0.024% to 0.040% per 1×10⁶ vc/mL, or about 0.028% to 0.047% per 1×10⁶ vc/mL, is effective to lyse the host cells, producing a host cell detergent lysate.

In some embodiments, the suspension of host cells and the lysis solution comprising detergent are mixed for a period of time during the addition of the lysis solution and/or after all lysis solution has been added, to effect thorough mixing of the two solutions. In some embodiments, such mixing can be performed for at least or about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 3 hrs, 4 hrs, 5 hrs, 6 hrs, or more, or a range including and between any two of the foregoing times, such as 15 mins to 90 mins, or some other range of time. In some embodiments, after mixing, the mixture of the host cell suspension and the detergent lysis solution are held, or incubated, for a period of time without active mixing. In some embodiments, the hold period can be at least or about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 3 hrs, 4 hrs, 5 hrs, 6 hrs, or more, or a range including and between any two of the foregoing times, such as 15 mins to 90 mins, or some other range of time. In some embodiments, the mixing and/or holding are performed at about room temperature, for example, 20 to 22° C., or some other temperature, such as about 2° C. to 8° C., 4° C., or 37° C.

Precipitating Host Cell DNA

Lysis of host cells releases host cell DNA into the surrounding fluid, for example growth medium, in which the cells are suspended. A significant proportion of the host cell DNA is genomic DNA, but can include any DNA released from a lysed host cell, for example mitochondrial DNA and/or plasmid DNA. Whilst a goal of the present methods is to reduce the amount of host cell DNA in a sample of lysed host cells, the methods may also be effective, in some embodiments, to at least partially remove RNA from a lysate, as well as proteins, such as histones, complexed with host cell DNA as chromatin. Host cell DNA can be removed from lysates of host cells by any technique known in the art to be effective to remove host cell DNA from a lysate. As used herein, removal of host cell DNA from lysates of host cells does not require removal of all such DNA, but merely reduction in the amount of host cell DNA in a portion of the lysate, as will be made clearer below.

In some embodiments, host cell DNA can be removed from a lysate by precipitating the host cell DNA by contacting the DNA with a cationic organic compound, such as a cationic detergent. Such contacting can conveniently be performed by adding a stock solution comprising a cationic detergent (i.e., DNA precipitation solution), and optionally other components such as buffers, to a host cell lysate in a suitable container to achieve a final concentration of the cationic detergent in the lysate (e.g., as a % weight by volume, % weight by weight, or molarity) sufficient to precipitate the host cell DNA, and then mixing to evenly distribute the cationic detergent throughout the lysate and contact the DNA. In some embodiments, the container in which DNA precipitation is performed is the same container, such as a bioreactor, in which host cell lysis was performed. DNA precipitation solution can be added to a bioreactor, or any container in which host cell DNA is to be precipitated as one or more boluses, or continually until the entire desired volume of DNA precipitation solution has been added, in any way that is known in the art, for example, from above, such as through a tube positioned above the fluid in which the host cells are suspended, or from below the surface of such fluid at any desired level of the bioreactor, such as through subsurface addition lines or tubes. Mixing can occur while DNA precipitation solution is being added, and/or for some period thereafter, in each case to thoroughly mix the lysate and DNA precipitation solutions together. Mixing can be performed in any way that is known the art, such as using impellers or pumps.

While not wishing to be bound by theory, it is believed that the positively charged groups in cationic detergents can interact electrostatically with the negatively charged phosphates in the DNA backbone, while hydrophobic groups within the detergent molecules interact non-covalently to exclude water, causing complexes of DNA and detergent molecules to precipitate. Initially, the particles are very small and remain suspended in solution, although they may be detectible by causing an increase in turbidity, which can be monitored if desired. As the precipitation reaction proceeds, however, ever larger particles of complexed DNA and detergent coalesce, eventually forming aggregates of sufficient size (flocs) that are able to settle out of the solution under the influence of gravity, forming a flocculant at the bottom of a containment vessel (e.g., a bottle, tank, or stirred-tank bioreactor, or the like) in which the precipitation reaction occurs, above which forms a partially clarified supernatant containing biological product, such as AAV vector. Typically, the flocculant is low density and easily disturbed, and so after mixing to distribute the cationic detergent in the lysate, the mixture is often held, or incubated, without significant mixing or agitation, or at most gentle mixing, to permit cationic detergent and DNA to interact to form a precipitate which settles out of solution as the flocculant.

In some embodiments, the cationic detergent is a quaternary ammonium compound, such as alkyl-dimethyl-(2-phenoxyethyl)azanium or its salt in which the charge on the quaternary ammonium cation is balanced by a halide, such as bromide, chloride, or iodide. In some embodiments, the cationic detergent is an alkyl-dimethyl-(2-phenoxyethyl)azanium halide, in which the alkyl substituent is C₆-C₁₈, or C₈-C₁₆, C₁₀-C₁₄, or C₁₂, and the halide is bromide, chloride, or iodide. In some embodiments, the cationic detergent is dodecyl-dimethyl-(2-phenoxyethyl)azanium, also called domiphen (CAS Registry Number 13900-14-6), and the halide is bromide, chloride, or iodide. In yet other embodiments, the cationic detergent is dodecyl-dimethyl-(2-phenoxyethyl)azanium bromide, also called domiphen bromide (CAS Registry Number 538-71-6), abbreviated “DB.”

In some embodiments, the final concentration (% weight by volume (w/v) or weight by weight (w/w), which are comparable for relatively dilute aqueous solutions) of the cationic detergent, such as alkyl-dimethyl-(2-phenoxyethyl)azanium halide, such as dodecyl-dimethyl-(2-phenoxyethyl)azanium halide, such as domiphen bromide (DB) in the mixture with the lysate is at least or about 0.05%, 0.10%, 0.15%, 0.20%, 0.25%, 0.30%, 0.35%, 0.40%, 0.45%, or 0.50%, or a range of concentrations including and between any two of the foregoing values, such as 0.10% to 0.50%, 0.10% to 0.40%, 0.10% to 0.30%, 0.10% to 0.20%, 0.15% to 0.45%, 0.20% to 0.50%, 0.20% to 0.40%, 0.20% to 0.30%, 0.25% to 0.35%, 0.25% to 0.45%, 0.30% to 0.50%, 0.30% to 0.40% to 0.50%, or some other range of concentrations.

In some embodiments, DB at a final concentration of about 0.3% is effective to precipitate host cell DNA from a detergent lysate of host cells lysed even at high viable cell densities at harvest, such as at least or about 9×10⁶ vc/mL, 10×10⁶ vc/mL, 11×10⁶ vc/mL, 12×10⁶ vc/mL, 13×10⁶ vc/mL, 14×10⁶ VC/M1_, 15×10⁶ vc/mL, 16×10⁶ vc/mL, 17×10⁶ vc/mL, 18×10⁶ vc/mL, 19×10⁶ vc/mL, 20×10⁶ vc/mL, 21×10⁶ VC/M1_, 22×10⁶ vc/mL, 23×10⁶ vc/mL, 24×10⁶ vc/mL, 25×10⁶ vc/mL, 26×10⁶ VC/M1_, 27×10⁶ vc/mL, 28×10⁶ vc/mL, 29×10⁶ vc/mL, 30×10⁶ vc/mL, or a range of viable cell density including and between any two of the foregoing values, such as from about 9×10⁶ vc/mL to 15×10⁶ VC/M1_, 10×10⁶ vc/mL to 14×10⁶ vc/mL, 18×10⁶ vc/mL to 24×10⁶ vc/mL, 10×10⁶ vc/mL to 30×10⁶ vc/mL, or 15×10⁶ vc/mL to 25×10⁶ vc/mL. In the latter two exemplary ranges, a final concentration of 0.3% DB is equivalent to 0.03% to 0.01% per 10⁶ viable host cells per mL, or to 0.02% to 0.012% per 10⁶ viable host cells per mL, respectively. These factors can be used to calculate final concentration ranges of DB that could be used to effectively precipitate host cell DNA at any particular viable cell density. For example, in some embodiments, DB at a final concentration of about 0.1% to 0.3%, or about 0.12% to 0.2%, is effective to precipitate host cell DNA at a viable cell density of about 10×10⁶ vc/mL, DB at a final concentration of about 0.2% to 0.6%, or about 0.24% to 0.4%, is effective to precipitate host cell DNA at a viable cell density of about 20×10⁶ vc/mL, and DB at a final concentration of about 0.3% to 0.9%, or about 0.36% to 0.6%, is effective to precipitate host cell DNA at a viable cell density of about 30×10⁶ vc/mL. In like fashion, in some embodiments, DB at a final concentration of about 0.6% to 1.8%, or about 0.72% to 1.2%, is effective to precipitate host cell DNA at a viable cell density of about 60×10⁶ vc/mL. In some embodiments, DB at a final concentration of not less than 0.009%, 0.008%, or 0.007% per 1×10⁶ vc/mL is effective to precipitate host cell DNA from a detergent lysate of host cells. In yet other embodiments related to any of the foregoing embodiments, the host cells can be HEK293 cells grown in suspension that produced an AAV vector, the amount of residual host cell DNA in the supernatant can be less than about 100, 90, 80, 70, 60, 50, 40, 30, or 20 picograms per 1×10⁹ vector genomes (pg/1×10⁹ vg), and the amount of AAV vector in the supernatant relative to the amount of AAV vector in the lysate (yield) can be at least about 30%, 35%, 40%, 45%, or 50%.

In the foregoing embodiments, the ratio of the final concentration of DB to the final concentration of Triton X-100 is about 0.6. In other embodiments, this ratio can vary somewhat without substantially reducing the yield of biological product, such as an AAV vector, and without substantially increasing the amount of residual host cell DNA in the supernatant. For example, in some embodiments, the final concentration of Triton X-100 can be about 0.3% and the final concentration of DB can vary from about 0.10% to 0.60% (or a DB to Triton X-100 ratio of about 0.33 to 2), 0.15% to 0.55% (or a DB to Triton X-100 ratio of about 0.5 to 1.83), 0.20% to 0.50% (or a DB to Triton X-100 ratio of about 0.67 to 1.67), 0.20% to 0.45% (or a DB to Triton X-100 ratio of about 0.67 to 1.5), 0.20% to 0.40% (or a DB to Triton X-100 ratio of about 0.67 to 1.33), 0.20% to 0.35% (or a DB to Triton X-100 ratio of about 0.67 to 1.17), 0.25% to 0.50% (or a DB to Triton X-100 ratio of about 0.83 to 1.67), 0.25% to 0.45% (or a DB to Triton X-100 ratio of about 0.83 to 1.5), 0.25% to 0.40% (or a DB to Triton X-100 ratio of about 0.83 to 1.33), 0.25% to 0.35% (or a DB to Triton X-100 ratio of about 0.83 to 1.17), be about 0.3% (or a DB to Triton X-100 ratio of about 1), or be about 0.2% (or a DB to Triton X-100 ratio of about 0.67). In any of the foregoing embodiments, the concentration of viable host cells at harvest can be at least or about 9×10⁶ vc/mL, 10×10⁶ VC/ML 11×10⁶ VC/ML 12×10⁶ VC/ML 13×10⁶ VC/ML 14×10⁶ VC/ML, 15×10⁶ vc/mL, 16×10⁶ vc/mL, 17×10⁶ vc/mL, 18×10⁶ VC/M1_, 19×10⁶ vc/mL, 20×10⁶ vc/mL, 21×10⁶ vc/mL, 22×10⁶ vc/mL, 23×10⁶ vc/mL, 24×10⁶ VC/M1_, 25×10⁶ vc/mL, 26×10⁶ vc/mL, 27×10⁶ vc/mL, 28×10⁶ vc/mL, 29×10⁶ vc/mL, 30×10⁶ vc/mL, or a range of viable cell density including and between any two of the foregoing values, such as from about 9×10⁶ vc/mL to 15×10⁶ vc/mL, 10×10⁶ vc/mL to 14×10⁶ vc/mL, 18×10⁶ vc/mL to 24×10⁶ vc/mL, 10×10⁶ vc/mL to 30×10⁶ vc/mL, or 15×10⁶ vc/mL to 25×10⁶ vc/mL. In some embodiments, DB at a final concentration of not less than 0.009%, 0.008%, or 0.007% per 1×10⁶ vc/mL is effective to precipitate host cell DNA from a detergent lysate of host cells. In yet other embodiments related to any of the foregoing embodiments, the host cells can be HEK293 cells grown in suspension that produced an AAV vector, the amount of residual host cell DNA in the supernatant can be less than about 100, 90, 80, 70, 60, 50, 40, 30, or 20 picograms per 1×10⁹ vector genomes (pg/1×10⁹ vg), and the amount of AAV vector in the supernatant relative to the amount of AAV vector in the lysate (yield) can be at least about 30%, 35%, 40%, 45%, or 50%.

In some other embodiments, the final concentration of Triton X-100 can be about 0.3% to 0.4%, or about 0.4%, and the final concentration of DB can vary from about 0.10% to 0.60% (or a DB to Triton X-100 ratio of about 0.25 to 1.5), 0.15% to 0.55% (or a DB to Triton X-100 ratio of about 0.38 to 1.38), 0.20% to 0.50% (or a DB to Triton X-100 ratio of about 0.5 to 1.25), 0.20% to 0.45% (or a DB to Triton X-100 ratio of about 0.5 to 1.13), 0.20% to 0.40% (or a DB to Triton X-100 ratio of about 0.5 to 1), 0.20% to 0.35% (or a DB to Triton X-100 ratio of about 0.5 to 0.88), 0.25% to 0.50% (or a DB to Triton X-100 ratio of about 0.63 to 1.15), 0.25% to 0.45% (or a DB to Triton X-100 ratio of about 0.63 to 1.13), 0.25% to 0.40% (or a DB to Triton X-100 ratio of about 0.63 to 1), 0.25% to 0.35% (or a DB to Triton X-100 ratio of about 0.63 to 0.88), be about 0.3% (or a DB to Triton X-100 ratio of about 0.75), or be about 0.2% (or a DB to Triton X-100 ratio of about 0.5). In any of the foregoing embodiments, the concentration of viable host cells at harvest can be at least or about 9×10⁶ VC/ML, 10×10⁶ vc/mL, 11×10⁶ vc/mL, 12×10⁶ vc/mL, 13×10⁶ vc/mL, 14×10⁶ vc/mL, 15×10⁶ vc/mL, 16×10⁶ VC/M1_, 17×10⁶ vc/mL, 18×10⁶ vc/mL, 19×10⁶ vc/mL, 20×10⁶ vc/mL, 21×10⁶ vc/mL, 22×10⁶ VC/M1_, 23×10⁶ vc/mL, 24×10⁶ vc/mL, 25×10⁶ vc/mL, 26×10⁶ vc/mL, 27×10⁶ vc/mL, 28×10⁶ vc/mL, 29×10⁶ vc/mL, 30×10⁶ vc/mL, or a range of viable cell density including and between any two of the foregoing values, such as from about 9×10⁶ vc/mL to 15×10⁶ vc/mL, 10×10⁶ VC/ML to 14×10⁶ vc/mL, 18×10⁶ vc/mL to 24×10⁶ vc/mL, 10×10⁶ vc/mL to 30×10⁶ vc/mL, or 15×10⁶ vc/mL to 25×10⁶ vc/mL. In some embodiments, DB at a final concentration of not less than 0.009%, 0.008%, or 0.007% per 1×10⁶ vc/mL is effective to precipitate host cell DNA from a detergent lysate of host cells. In yet other embodiments related to any of the foregoing embodiments, the host cells can be HEK293 cells grown in suspension that produced an AAV vector, the amount of residual host cell DNA in the supernatant can be less than about 100, 90, 80, 70, 60, 50, 40, 30, or 20 picograms per 1×10⁹ vector genomes (pg/1×10⁹ vg), and the amount of AAV vector in the supernatant relative to the amount of AAV vector in the lysate (yield) can be at least about 30%, 35%, 40%, 45%, or 50%.

In some other embodiments, the final concentration of Triton X-100 can be about 0.4% to 0.5%, or about 0.5%, and the final concentration of DB can vary from about 0.10% to 0.60% (or a DB to Triton X-100 ratio of about 0.2 to 1.2), 0.15% to 0.55% (or a DB to Triton X-100 ratio of about 0.3 to 1.1), 0.20% to 0.50% (or a DB to Triton X-100 ratio of about 0.4 to 1), 0.20% to 0.45% (or a DB to Triton X-100 ratio of about 0.4 to 0.9), 0.20% to 0.40% (or a DB to Triton X-100 ratio of about 0.4 to 0.8), 0.20% to 0.35% (or a DB to Triton X-100 ratio of about 0.4 to 0.7), 0.25% to 0.50% (or a DB to Triton X-100 ratio of about 0.5 to 1), 0.25% to 0.45% (or a DB to Triton X-100 ratio of about 0.5 to 0.9), 0.25% to 0.40% (or a DB to Triton X-100 ratio of about 0.5 to 0.8), 0.25% to 0.35% (or a DB to Triton X-100 ratio of about 0.5 to 0.7), be about 0.3% (or a DB to Triton X-100 ratio of about 0.6), or be about 0.2% (or a DB to Triton X-100 ratio of about 0.4). In any of the foregoing embodiments, the concentration of viable host cells at harvest can be at least or about 9×10⁶ VC/ML 10×10⁶ VC/ML, 11×10⁶ vc/mL, 12×10⁶ vc/mL, 13×10⁶ vc/mL, 14×10⁶ vc/mL, 15×10⁶ vc/mL, 16×10⁶ vc/mL, 17×10⁶ VC/M1_, 18×10⁶ vc/mL, 19×10⁶ vc/mL, 20×10⁶ vc/mL, 21×10⁶ vc/mL, 22×10⁶ vc/mL, 23×10⁶ VC/M1_, 24×10⁶ vc/mL, 25×10⁶ vc/mL, 26×10⁶ vc/mL, 27×10⁶ vc/mL, 28×10⁶ vc/mL, 29×10⁶ vc/mL, 30×10⁶ vc/mL, or a range of viable cell density including and between any two of the foregoing values, such as from about 9×10⁶ vc/mL to 15×10⁶ vc/mL, 10×10⁶ VC/ML to 14×10⁶ vc/mL, 18×10⁶ vc/mL to 24×10⁶ vc/mL, 10×10⁶ vc/mL to 30×10⁶ vc/mL, or 15×10⁶ vc/mL to 25×10⁶ vc/mL. In some embodiments, DB at a final concentration of not less than 0.009%, 0.008%, or 0.007% per 1×10⁶ vc/mL is effective to precipitate host cell DNA from a detergent lysate of host cells. In yet other embodiments related to any of the foregoing embodiments, the host cells can be HEK293 cells grown in suspension that produced an AAV vector, the amount of residual host cell DNA in the supernatant can be less than about 100, 90, 80, 70, 60, 50, 40, 30, or 20 picograms per 1×10⁹ vector genomes (pg/1×10⁹ vg), and the amount of AAV vector in the supernatant relative to the amount of AAV vector in the lysate (yield) can be at least about 30%, 35%, 40%, 45%, or 50%.

In some other embodiments, the final concentration of Triton X-100 can be about 0.5% to 0.6%, or about 0.6%, and the final concentration of DB can vary from about 0.10% to 0.60% (or a DB to Triton X-100 ratio of about 0.17 to 1), 0.15% to 0.55% (or a DB to Triton X-100 ratio of about 0.25 to 0.92), 0.20% to 0.50% (or a DB to Triton X-100 ratio of about 0.33 to 0.83), 0.20% to 0.45% (or a DB to Triton X-100 ratio of about 0.33 to 0.75), 0.20% to 0.40% (or a DB to Triton X-100 ratio of about 0.33 to 0.67), 0.20% to 0.35% (or a DB to Triton X-100 ratio of about 0.33 to 0.58), 0.25% to 0.50% (or a DB to Triton X-100 ratio of about 0.42 to 0.83), 0.25% to 0.45% (or a DB to Triton X-100 ratio of about 0.42 to 0.75), 0.25% to 0.40% (or a DB to Triton X-100 ratio of about 0.42 to 0.67), 0.25% to 0.35% (or a DB to Triton X-100 ratio of about 0.42 to 0.58), be about 0.3% (or a DB to Triton X-100 ratio of about 0.5), or be about 0.2% (or a DB to Triton X-100 ratio of about 0.33). In any of the foregoing embodiments, the concentration of viable host cells at harvest can be at least or about 9×10⁶ vc/mL, 10×10⁶ vc/mL, 11×10⁶ vc/mL, 12×10⁶ vc/mL, 13×10⁶ vc/mL, 14×10⁶ vc/mL, 15×10⁶ vc/mL, 16×10⁶ vc/mL, 17×10⁶ vc/mL, 18×10⁶ vc/mL, 19×10⁶ vc/mL, 20×10⁶ vc/mL, 21×10⁶ vc/mL, 22×10⁶ vc/mL, 23×10⁶ vc/mL, 24×10⁶ vc/mL, 25×10⁶ vc/mL, 26×10⁶ vc/mL, 27×10⁶ vc/mL, 28×10⁶ vc/mL, 29×10⁶ vc/mL, 30×10⁶ vc/mL, or a range of viable cell density including and between any two of the foregoing values, such as from about 9×10⁶ vc/mL to 15×10⁶ vc/mL, 10×10⁶ vc/mL to 14×10⁶ vc/mL, 18×10⁶ vc/mL to 24×10⁶ vc/mL, 10×10⁶ vc/mL to 30×10⁶ vc/mL, or 15×10⁶ vc/mL to 25×10⁶ vc/mL. In some embodiments, DB at a final concentration of not less than 0.009%, 0.008%, or 0.007% per 1×10⁶ vc/mL is effective to precipitate host cell DNA from a detergent lysate of host cells. In yet other embodiments related to any of the foregoing embodiments, the host cells can be HEK293 cells grown in suspension that produced an AAV vector, the amount of residual host cell DNA in the supernatant can be less than about 100, 90, 80, 70, 60, 50, 40, 30, or 20 picograms per 1×10⁹ vector genomes (pg/1×10⁹ vg), and the amount of AAV vector in the supernatant relative to the amount of AAV vector in the lysate (yield) can be at least about 30%, 35%, 40%, 45%, or 50%.

In some other embodiments, the final concentration of Triton X-100 can be about 0.6% to 0.7%, or about 0.7%, and the final concentration of DB can vary from about 0.10% to 0.60% (or a DB to Triton X-100 ratio of about 0.14 to 0.86), 0.15% to 0.55% (or a DB to Triton X-100 ratio of about 0.21 to 0.79), 0.20% to 0.50% (or a DB to Triton X-100 ratio of about 0.29 to 0.71), 0.20% to 0.45% (or a DB to Triton X-100 ratio of about 0.29 to 0.64), 0.20% to 0.40% (or a DB to Triton X-100 ratio of about 0.29 to 0.57), 0.20% to 0.35% (or a DB to Triton X-100 ratio of about 0.29 to 0.5), 0.25% to 0.50% (or a DB to Triton X-100 ratio of about 0.36 to 0.71), 0.25% to 0.45% (or a DB to Triton X-100 ratio of about 0.36 to 0.64), 0.25% to 0.40% (or a DB to Triton X-100 ratio of about 0.36 to 0.57), 0.25% to 0.35% (or a DB to Triton X-100 ratio of about 0.36 to 0.5), be about 0.3% (or a DB to Triton X-100 ratio of about 0.43), or be about 0.2% (or a DB to Triton X-100 ratio of about 0.29). In any of the foregoing embodiments, the concentration of viable host cells at harvest can be at least or about 9×10⁶ vc/mL, 10×10⁶ vc/mL, 11×10⁶ vc/mL, 12×10⁶ vc/mL, 13×10⁶ vc/mL, 14×10⁶ vc/mL, 15×10⁶ VC/M1_, 16×10⁶ vc/mL, 17×10⁶ vc/mL, 18×10⁶ vc/mL, 19×10⁶ vc/mL, 20×10⁶ vc/mL, 21×10⁶ vc/mL, 22×10⁶ VC/M1_, 23×10⁶ vc/mL, 24×10⁶ vc/mL, 25×10⁶ vc/mL, 26×10⁶ vc/mL, 27×10⁶ vc/mL, 28×10⁶ vc/mL, 29×10⁶ vc/mL, 30×10⁶ vc/mL, or a range of viable cell density including and between any two of the foregoing values, such as from about 9×10⁶ vc/mL to 15×10⁶ vc/mL, 10×10⁶ VC/ML to 14×10⁶ vc/mL, 18×10⁶ vc/mL to 24×10⁶ vc/mL, 10×10⁶ vc/mL to 30×10⁶ vc/mL, or 15×10⁶ vc/mL to 25×10⁶ vc/mL. In some embodiments, DB at a final concentration of not less than 0.009%, 0.008%, or 0.007% per 1×10⁶ vc/mL is effective to precipitate host cell DNA from a detergent lysate of host cells. In yet other embodiments related to any of the foregoing embodiments, the host cells can be HEK293 cells grown in suspension that produced an AAV vector, the amount of residual host cell DNA in the supernatant can be less than about 100, 90, 80, 70, 60, 50, 40, 30, or 20 picograms per 1×10⁹ vector genomes (pg/1×10⁹ vg), and the amount of AAV vector in the supernatant relative to the amount of AAV vector in the lysate (yield) can be at least about 30%, 35%, 40%, 45%, or 50%.

In some other embodiments, DB added to a detergent lysate of host cells, such as HEK293 cells grown in suspension culture, at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 added to a sample of host cells, such as HEK293 cells grown in suspension culture, at a final concentration of about 0.010% to 0.030% per 1×10⁶ vc/mL is effective to lyse the host cells, producing a host cell detergent lysate, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 at a final concentration of about 0.012% to 0.020% per 1×10⁶ vc/mL is effective to lyse the host cells, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 at a final concentration of about 0.013% to 0.040% per 1×10⁶ vc/mL is effective to lyse the host cells, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 at a final concentration of about 0.016% to 0.027% per 1×10⁶ vc/mL is effective to lyse the host cells, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 at a final concentration of about 0.017% to 0.050% per 1×10⁶ vc/mL is effective to lyse the host cells, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 at a final concentration of about 0.020% to 0.033% per 1×10⁶ vc/mL is effective to lyse the host cells, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 at a final concentration of about 0.020% to 0.060% per 1×10⁶ vc/mL is effective to lyse the host cells, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 at a final concentration of about 0.023% to 0.070% per 1×10⁶ vc/mL is effective to lyse the host cells, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 at a final concentration of about 0.024% to 0.040% per 1×10⁶ vc/mL is effective to lyse the host cells, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate. In some embodiments, Triton X-100 at a final concentration of about 0.028% to 0.047% per 1×10⁶ vc/mL is effective to lyse the host cells, and DB at a final concentration of about 0.003% to 0.010% per 1×10⁶ vc/mL, 0.004% to 0.007% per 1×10⁶ vc/mL, 0.007% to 0.020% per 1×10⁶ vc/mL, 0.008% to 0.013% per 1×10⁶ vc/mL, 0.010% to 0.030% per 1×10⁶ vc/mL, 0.012% to 0.020% per 1×10⁶ vc/mL, 0.013% to 0.040% per 1×10⁶ vc/mL, 0.016% to 0.027% per 1×10⁶ vc/mL, 0.017% to 0.050% per 1×10⁶ vc/mL, 0.020% to 0.033% per 1×10⁶ vc/mL, is effective to precipitate host cell DNA from the host cell detergent lysate.

In some embodiments, the viable cell density at harvest ranges from about 10×10⁶ vc/mL to 30×10⁶ vc/mL, 15×10⁶ vc/mL to 25×10⁶ vc/mL, the cells produce AAV vector, the final concentration of Triton X-100 used to lyse the cells ranges from about 0.35% to 0.65%, or 0.4% to 0.6%, the final concentration of DB used to precipitate host cell DNA ranges from about 0.15% to 0.45%, or 0.2% to 0.4%, the amount of residual host cell DNA in the supernatant is less than about 100, 90, 80, 70, 60, 50, 40, 30, or 20 picograms per 1×10⁹ vector genomes (pg/1×10⁹ vg), and the amount of AAV vector in the supernatant relative to the amount of AAV vector in the lysate (yield) is at least about 30%, 35%, 40%, 45%, or 50%. In any of these embodiments, the host cells can be HEK293 cells grown in suspension culture.

In some embodiments, the viable cell density at harvest ranges from about 15×10⁶ vc/mL to 25×10⁶ vc/mL, the cells produce AAV vector, the final concentration of Triton X-100 used to lyse the cells ranges from about 0.4% to 0.6%, the final concentration of DB used to precipitate host cell DNA ranges from about 0.2% to 0.4%, the amount of residual host cell DNA in the supernatant is less than about 50 picograms per 1×10⁹ vector genomes (pg/1×10⁹ vg), and the amount of AAV vector in the supernatant relative to the amount of AAV vector in the lysate (yield) is at least about 40%. In any of these embodiments, the host cells can be HEK293 cells grown in suspension culture.

In some embodiments, the viable cell density at harvest ranges from about 15×10⁶ vc/mL to 25×10⁶ vc/mL, the cells produce AAV vector, the final concentration of Triton X-100 used to lyse the cells is about 0.5%, the final concentration of DB used to precipitate host cell DNA ranges from about 0.2% to 0.3%, the amount of residual host cell DNA in the supernatant is less than about 50 picograms per 1×10⁹ vector genomes (pg/1×10⁹ vg), and the amount of AAV vector in the supernatant relative to the amount of AAV vector in the lysate (yield) is at least about 40%. In any of these embodiments, the host cells can be HEK293 cells grown in suspension culture.

In some embodiments, the host cell lysate and the DNA precipitation solution comprising the cationic detergent, such as DB, are mixed for a period of time during the addition of the DNA precipitation solution and/or after all DNA precipitation solution has been added, to effect thorough mixing of the two solutions. In some embodiments, such mixing can be performed for at least or about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 180 mins, or a range of time including and between any two of the foregoing values, such as 15 mins to 60 mins, or some other range of time. In some embodiments, after mixing, the mixture of the host cell lysate and the DNA precipitation solution comprising the cationic detergent, such as DB, is held for a period of time without active mixing to permit settling of flocs. In some embodiments, the hold period can be at least or about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 3 hrs, 4 hrs, 5 hrs, 6 hrs, or more, or a range including and between any two of the foregoing times, such as 30 mins to 3 hrs, 1 hr to 6 hrs, or some other range of time. In some embodiments, the mixing and/or holding are performed at about room temperature, for example, about 20° C. to 22° C., or some other temperature, such as about 2° C. to 8° C., 4° C., or 37° C.

Separating Flocculated Host Cell DNA from Supernatant

Flocculated host cell DNA can be separated from supernatant by any technique known in the art to be effective to separate flocculated host cell DNA and a supernatant. As noted above, in some embodiments, flocculated host cell DNA can be separated from supernatant by allowing flocs to settle under the influence of gravity for a period of time to the bottom of a container in which host cell lysate and DNA precipitation solution were mixed, usually without mixing while settling is occurring. Alternatively, the flocculated host cell DNA can be separated from the supernatant by centrifugation, such as by continuous flow centrifugation. Flocs can also be removed from the mixture through one or more depth filters.

Once partially clarified supernatant (lysate) has formed above the layer of flocculated host cell DNA, the supernatant or the flocculant can be removed by pumping. For example, the supernatant can be pumped out through a tube inserted from above, the end of which is immersed in the supernatant but positioned above the flocculant layer, or through a port inserted through a wall of the container located above the layer of flocculant. Alternatively, the flocculant can be pumped out through a tube inserted from above, the end of which is immersed in the flocculant, or through a port at the bottom of the container or inserted through a wall of the container located below the supernatant. A combination of these methods can also be used. Typically, after being removed or separated from the flocculated host cell DNA, the supernatant is transferred to a new container.

In some embodiments, the partially clarified supernatant, after having been removed or separated from the flocculated host cell DNA, such as by pumping, is filtered to remove any flocs that may have been carried along during the removal process, for example by filtering through one or more depth filters and/or membrane filters. Filtering the partially clarified supernatant (lysate) results in a clarified supernatant (lysate). In some embodiments, one or more of the filters has a nominal retention rating, or average pore size, of less than or equal to about 100 μm, 50 μm, 40 μm, 30 μm, 25 μm, 20 μm, 15 μm, 10 μm, 5 μm, 2 μm, 1 μm, or 0.5 μm.

Inhibiting Precipitation of Residual Host Cell DNA

Although the step of precipitating host cell DNA will often be highly effective to remove a significant proportion of DNA from the crude host cell detergent lysate, it is expected that some residual and uncomplexed host cell DNA and cationic detergent can remain in the partially clarified or clarified supernatant (lysate) from the prior steps, particularly when the host cells were grown or maintained to a high viable cell density while the desired biological product was being produced within the cells and just prior to their lysis. With no intervention, the residual DNA and detergent, such as DB, can continue to complex, although usually at a slower rate due to their lower concentration, gradually forming particles of sufficient size to be detected as increasing turbidity with time. Even when the increase in turbidity is of modest extent, this ongoing process can produce a sufficient concentration of such particles to reduce the efficiency, sometimes severely, of downstream processing steps intended to further purify the desired biological product, such as AAV vectors. To contend with this issue, in some embodiments, the methods of the disclosure further comprise a step of inhibiting precipitation of residual host cell DNA in the supernatant.

Precipitation of residual host cell DNA in the supernatant can be inhibited using any technique known in the art to be effective to inhibit precipitation of residual host cell DNA in a supernatant. In some embodiments, precipitation of residual host cell DNA in the supernatant is inhibited by adding to the supernatant an amount of a salt sufficient to inhibit precipitation of the host cell DNA, for example by a domiphen halide, such as DB. In some embodiments, the salt is sodium chloride (NaCl), potassium chloride (KCl), magnesium sulfate (MgSO₄), or magnesium chloride (MgCl₂). In some embodiments, the salt can be added to the supernatant in solid form, or in a concentrated stock solution comprising the salt (i.e., salt solution), and optionally other ingredients, such as detergent, such as Triton X-100, to any desired final concentration, for example in an amount sufficient to achieve a final concentration in the supernatant of about 0.5% (w/v or w/w).

In some embodiments, the supernatant and the solution comprising the salt are mixed for a period of time to effect thorough mixing of the two solutions, forming a mixture, and then optionally filtered and/or held (incubated) for a period time, such as in storage, without mixing. In some embodiments, the mixing and/or holding are performed at about room temperature, for example, about 20° C. to 22° C., or some other temperature, such as about 2° C. to 8° C., 4° C., or 37° C. In some embodiments, the mixing of the supernatant and the solution comprising the salt is performed for at least or about 5 mins, 10 mins, 15 mins, 20 mins, 30 mins, 40 mins, 50 mins, 60 mins, 70 mins, 75 mins, 80 mins, 90 mins, 100 mins, 115 mins, 120 mins, 150 mins, 180 mins, or a range of time including and between any two of the foregoing values, such as 15 mins to 60 mins, or some other range of time.

In some embodiments, the step of inhibiting precipitation of residual host cell DNA in the supernatant, for example by a domiphen halide, such as DB, is performed shortly after the prior step of removing or separating the supernatant from the flocculated host cell DNA, and optionally filtering the supernatant. In some embodiments, the delay between the conclusion of the step of removing and optionally filtering supernatant and commencing the step of inhibiting precipitation is less than about 12 hrs, 6 hrs, 3 hrs, 2 hrs, 1 hr, 45 mins, 30 mins, 15 mins, 10 mins, 5 mins, or less time.

In some embodiments, the final concentration of added salt, for example of NaCl, KCl, MgSO₄, or MgCl₂, or another added salt, in the supernatant after its addition is at least or about 5 mM, 10 mM, 20 mM, 30 mM, 40 mM, 50 mM, 75 mM, 100 mM, 125 mM, 150 mM, 175 mM, 200 mM, 225 mM, 250 mM, 300 mM, 350 mM, 400 mM, 450 mM, 500 mM, 550 mM, 600 mM, 650 mM, 700 mM, 750 mM, or 800 mM, or a range of concentrations including and between any two of the foregoing values, such as 10 mM to 200 mM, 10 mM to 400 mM, 200 mM to 600 mM, 200 mM to 800 mM, 300 mM to 500 mM, 300 mM to 600 mM, 250 mM to 600 mM, 250 mM to 400 mM, or 400 mM to 600 mM. In some embodiments, the host cell DNA is precipitated with a domiphen halide, such as DB.

For clarity, it is to be noted that the final concentration of an added salt as described herein refers to the final concentration of the salt that is added without regard to the concentration of the same type of salt that may pre-exist in the supernatant. For example, as is well known in the art, cell culture media of various kinds already include salts, such as NaCl and KCl, and the concentration of such salts already present is not included in the value of the final concentration of added salt, unless otherwise indicated.

In some embodiments, the salt is NaCl or KCl, and the final concentration of added salt in the supernatant is at least or about 100 mM, 125 mM, 150 mM, 175 mM, 200 mM, 225 mM, 250 mM, 300 mM, 350 mM, 400 mM, 450 mM, 500 mM, 550 mM, or 600 mM, or a range of concentrations including and between any two of the foregoing values, such as about 200 mM to 300 mM, 200 mM to 400 mM, 200 mM to 500 mM, 200 mM to 600 mM, 200 mM to 700 mM, 200 mM to 800 mM, 300 mM to 500 mM, 300 mM to 600 mM, 250 mM to 600 mM, 250 mM to 400 mM, 250 mM to 350 mM, or 400 to 600 mM. In some embodiments, the salt is MgSO₄, and the final concentration of added salt in the treated supernatant is at least or about 10 mM, 50 mM, 100 mM, 200 mM, 300 mM, or 400 mM, whereas in other embodiments, the salt is MgCl₂, and the final concentration of added salt in the treated supernatant is at least or about 1 mM, 5 mM, 10 mM, 20 mM, 50 mM, or 100 mM. In some embodiments, the host cell DNA is precipitated with a domiphen halide, such as DB.

In some embodiments, the host cell DNA is precipitated with a domiphen halide, such as DB, and the DB at a final concentration of about 0.1% to 0.5%, or about 0.2% to 0.4%, such as about 0.2% or 0.3%, is effective to precipitate host cell DNA when added to a detergent lysate of host cells lysed even at high viable cell densities at harvest, such as at least about 10×10⁶ vc/mL, least about 15×10⁶ vc/mL, least about 20×10⁶ vc/mL, or about 10×10⁶ vc/mL to 30×10⁶ vc/mL, or 10×10⁶ vc/mL to 25×10⁶ vc/mL, and subsequent addition of a salt to the supernatant, such as NaCl or KCl, to a final concentration of added salt of at least about 200 mM, 300 mM, 400 mM, 500 mM, 600 mM, 700 mM, 800 mM, or in a range of about 200 mM to 800 mM, is effective to inhibit, or quench, precipitation of residual host cell DNA in the mixture. Such quenching can usefully enhance the efficiency of downstream processing steps intended to further purify a biological product, such as an AAV vector, for example by reducing column fouling during a downstream chromatography step, such as immunoaffinity chromatography.

In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 10×⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 10×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 10×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 10×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 10×10⁶ vc/mL, and NaCl or KCl can be added to the supernatant to a final added salt concentration of about 600 mM, or about 600 mM to 700 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 10×10⁶ vc/mL, and NaCl or KCl can be added to the supernatant to a final added salt concentration of about 700 mM, or about 700 mM to 800 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 10×10⁶ vc/mL, and NaCl or KCl can be added to the supernatant to a final added salt concentration of about 200 mM to 300 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 15×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 15×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 15×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 15×10⁶ vc/mL, and NaCl or KCl can be added to the supernatant to a final added salt concentration of about 600 mM, or about 600 mM to 700 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 15×10⁶ vc/mL, and NaCl or KCl can be added to the supernatant to a final added salt concentration of about 700 mM, or about 700 mM to 800 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 20×10⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 20×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 20×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 20×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 20×10⁶ vc/mL, and NaCl or KCl can be added to the supernatant to a final added salt concentration of about 600 mM, or about 600 mM to 700 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is at least about 20×10⁶ vc/mL, and NaCl or KCl can be added to the supernatant to a final added salt concentration of about 700 mM, or about 700 mM to 800 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 10×10⁶ vc/mL to 30×10⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 10×10⁶ vc/mL to 30×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 10×10⁶ vc/mL to 30×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 10×10⁶ vc/mL to 30×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 10×10⁶ vc/mL to 30×10⁶ vc/mL, and NaCl or KCl can be added to the supernatant to a final added salt concentration of about 600 mM, or about 600 mM to 700 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 10×10⁶ vc/mL to 30×10⁶ vc/mL, and NaCl or KCl can be added to the supernatant to a final added salt concentration of about 700 mM, or about 700 mM to 800 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 15×10⁶ vc/mL to 25×10⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 15×10⁶ vc/mL to 25×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 15×10⁶ vc/mL to 25×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 15×10⁶ vc/mL to 25×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 15×10⁶ vc/mL to 25×10⁶ vc/mL, and NaCl or KCl can be added to the supernatant to a final added salt concentration of about 600 mM, or about 600 mM to 700 mM to inhibit precipitation of residual host cell DNA. In some embodiments, DB can be added to a detergent lysate of host cells to a final concentration of about 0.1% to 0.5%, or about 0.2% to 0.4%, such as about 0.2% or 0.3%, to precipitate host cell DNA from the lysate, where the viable cell density at harvest is about 15×10⁶ vc/mL to 25×10⁶ vc/mL, and NaCl or KCl can be added to the supernatant to a final added salt concentration of about 700 mM, or about 700 mM to 800 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.2% to precipitate host cell DNA from the lysate, where the viable cell density at harvest is about 10×10⁶ vc/mL to 30×10⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.2% to precipitate host cell DNA from the lysate, where the viable cell density at harvest is about 10×10⁶ vc/mL to 30×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.2% to precipitate host cell DNA from the lysate, where the viable cell density at harvest is about 10×10⁶ vc/mL to 30×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.2% to precipitate host cell DNA from the lysate, where the viable cell density at harvest is about 10×10⁶ vc/mL to 30×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.2% to precipitate host cell DNA from the lysate, where the viable cell density at harvest is about 10×10⁶ vc/mL to 30×10⁶ vc/mL, and NaCl or KCl can be added to the supernatant to a final added salt concentration of about 600 mM, or about 600 mM to 700 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.3% to precipitate host cell DNA from the lysate, where the viable cell density at harvest is about 10×10⁶ vc/mL to 30×10⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.3% to precipitate host cell DNA from the lysate, where the viable cell density at harvest is about 10×10⁶ vc/mL to 30×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.3% to precipitate host cell DNA from the lysate, where the viable cell density at harvest is about 10×10⁶ vc/mL to 30×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.3% to precipitate host cell DNA from the lysate, where the viable cell density at harvest is about 10×10⁶ vc/mL to 30×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.3% to precipitate host cell DNA from the lysate, where the viable cell density at harvest is about 10×10⁶ vc/mL to 30×10⁶ vc/mL, and NaCl or KCl can be added to the supernatant to a final added salt concentration of about 600 mM, or about 600 mM to 700 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.4% to precipitate host cell DNA from the lysate, where the viable cell density at harvest is about 10×10⁶ vc/mL to 30×10⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.4% to precipitate host cell DNA from the lysate, where the viable cell density at harvest is about 10×10⁶ vc/mL to 30×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.4% to precipitate host cell DNA from the lysate, where the viable cell density at harvest is about 10×10⁶ vc/mL to 30×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.4% to precipitate host cell DNA from the lysate, where the viable cell density at harvest is about 10×10⁶ vc/mL to 30×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.4% to precipitate host cell DNA from the lysate, where the viable cell density at harvest is about 10×10⁶ vc/mL to 30×10⁶ vc/mL, and NaCl or KCl can be added to the supernatant to a final added salt concentration of about 600 mM, or about 600 mM to 700 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.2% to precipitate host cell DNA from the lysate, where the viable cell density at harvest is about 15×10⁶ vc/mL to 25×10⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.2% to precipitate host cell DNA from the lysate, where the viable cell density at harvest is about 15×10⁶ vc/mL to 25×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.2% to precipitate host cell DNA from the lysate, where the viable cell density at harvest is about 15×10⁶ vc/mL to 25×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.2% to precipitate host cell DNA from the lysate, where the viable cell density at harvest is about 15×10⁶ vc/mL to 25×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.2% to precipitate host cell DNA from the lysate, where the viable cell density at harvest is about 15×10⁶ vc/mL to 25×10⁶ vc/mL, and NaCl or KCl can be added to the supernatant to a final added salt concentration of about 600 mM, or about 600 mM to 700 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.3% to precipitate host cell DNA from the lysate, where the viable cell density at harvest is about 15×10⁶ vc/mL to 25×10⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.3% to precipitate host cell DNA from the lysate, where the viable cell density at harvest is about 15×10⁶ vc/mL to 25×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.3% to precipitate host cell DNA from the lysate, where the viable cell density at harvest is about 15×10⁶ vc/mL to 25×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.3% to precipitate host cell DNA from the lysate, where the viable cell density at harvest is about 15×10⁶ vc/mL to 25×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.3% to precipitate host cell DNA from the lysate, where the viable cell density at harvest is about 15×10⁶ vc/mL to 25×10⁶ vc/mL, and NaCl or KCl can be added to the supernatant to a final added salt concentration of about 600 mM, or about 600 mM to 700 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, DB can be added to a detergent lysate of host cells to a final concentration of about 0.4% to precipitate host cell DNA from the lysate, where the viable cell density at harvest is about 15×10⁶ vc/mL to 25×10⁶ vc/mL, and NaCl or KCl can be added to the supernatant to a final added salt concentration of about 200 mM, or about 200 mM to 300 mM to inhibit precipitation of residual host cell DNA. In some embodiments, DB can be added to a detergent lysate of host cells to a final concentration of about 0.4% to precipitate host cell DNA from the lysate, where the viable cell density at harvest is about 15×10⁶ vc/mL to 25×10⁶ vc/mL, and NaCl or KCl can be added to the supernatant to a final added salt concentration of about 300 mM, or about 300 mM to 400 mM to inhibit precipitation of residual host cell DNA. In some embodiments, DB can be added to a detergent lysate of host cells to a final concentration of about 0.4% to precipitate host cell DNA from the lysate, where the viable cell density at harvest is about 15×10⁶ vc/mL to 25×10⁶ vc/mL, and NaCl or KCl can be added to the supernatant to a final added salt concentration of about 400 mM, or about 400 mM to 500 mM to inhibit precipitation of residual host cell DNA. In some embodiments, DB can be added to a detergent lysate of host cells to a final concentration of about 0.4% to precipitate host cell DNA from the lysate, where the viable cell density at harvest is about 15×10⁶ vc/mL to 25×10⁶ vc/mL, and NaCl or KCl can be added to the supernatant to a final added salt concentration of about 500 mM, or about 500 mM to 600 mM to inhibit precipitation of residual host cell DNA. In some embodiments, DB can be added to a detergent lysate of host cells to a final concentration of about 0.4% to precipitate host cell DNA from the lysate, where the viable cell density at harvest is about 15×10⁶ vc/mL to 25×10⁶ vc/mL, and NaCl or KCl can be added to the supernatant to a final added salt concentration of about 600 mM, or about 600 mM to 700 mM to inhibit precipitation of residual host cell DNA. In any of the foregoing embodiments, the host cells can be lysed with Triton X-100 at a final concentration ranging from about 0.3% to 0.7%, or 0.4% to 0.6%, or about 0.5%, and the host cells can be HEK293 cells grown in suspension culture and which produce AAV vector.

In some embodiments, HEK293 cells grown in suspension culture producing an AAV vector are harvested at a viable cell density of at least or about 10×10⁶ vc/mL by adding Triton X-100 to a final concentration of at least or about 0.5% to lyse the cells, DB is added to a final concentration of at least or about 0.2% to precipitate host cell DNA from the detergent lysate, and NaCl or KCl is added to the supernatant to a final added salt concentration of at least or about 200 mM to inhibit precipitation of residual host cell DNA. In some embodiments, HEK293 cells grown in suspension culture producing an AAV vector are harvested at a viable cell density of at least or about 15×10⁶ vc/mL by adding Triton X-100 to a final concentration of at least or about 0.5% to lyse the cells, DB is added to a final concentration of at least or about 0.2% to precipitate host cell DNA from the detergent lysate, and NaCl or KCl is added to the supernatant to a final added salt concentration of at least or about 200 mM to inhibit precipitation of residual host cell DNA. In some embodiments, HEK293 cells grown in suspension culture producing an AAV vector are harvested at a viable cell density of at least or about 20×10⁶ vc/mL by adding Triton X-100 to a final concentration of at least or about 0.5% to lyse the cells, DB is added to a final concentration of at least or about 0.2% to precipitate host cell DNA from the detergent lysate, and NaCl or KCl is added to the supernatant to a final added salt concentration of at least or about 200 mM to inhibit precipitation of residual host cell DNA. In some embodiments, HEK293 cells grown in suspension culture producing an AAV vector are harvested at a viable cell density of about 10×10⁶ vc/mL to 30 vc/mL by adding Triton X-100 to a final concentration of at least or about 0.5% to lyse the cells, DB is added to a final concentration of at least or about 0.2% to precipitate host cell DNA from the detergent lysate, and NaCl or KCl is added to the supernatant to a final added salt concentration of at least or about 200 mM to inhibit precipitation of residual host cell DNA. In some embodiments, HEK293 cells grown in suspension culture producing an AAV vector are harvested at a viable cell density of about 15×10⁶ vc/mL to 25×10⁶ vc/mL by adding Triton X-100 to a final concentration of at least or about 0.5% to lyse the cells, DB is added to a final concentration of at least or about 0.2% to precipitate host cell DNA from the detergent lysate, and NaCl or KCl is added to the supernatant to a final added salt concentration of at least or about 200 mM to inhibit precipitation of residual host cell DNA. In yet other embodiments related to any of the foregoing embodiments, the amount of residual host cell DNA in the supernatant can be less than about 50 picograms per 1×10⁹ vector genomes (pg/1×10⁹ vg), and the amount of AAV vector in the supernatant relative to the amount of AAV vector in the lysate (i.e., the yield) can be at least about 40%.

In some embodiments, the concentration of NaCl or KCl added to a supernatant prepared from detergent lysed host cells (e.g., HEK293 cells grown in suspension culture) from which host cell DNA was precipitated by adding DB to the crude lysate that is effective to inhibit precipitation of residual host cell DNA by DB can be expressed in terms of the amount of salt added relative to the viable cell density of the host cells (e.g., number of viable cells per milliliter (vc/mL) of the fluid in which the cells were suspended) at the time when they were lysed. Thus, for example, in some embodiments, the final concentration of NaCl or KCl added to a supernatant that is effective to inhibit precipitation of residual host cell DNA by DB is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL. In some embodiments, the final concentration of NaCl or KCl added to a supernatant that is effective to inhibit precipitation of residual host cell DNA by DB which had been added to the crude lysate at a final concentration of about 0.003% to 0.010% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL. In some embodiments, the final concentration of NaCl or KCl added to a supernatant that is effective to inhibit precipitation of residual host cell DNA by DB which had been added to the crude lysate at a final concentration of about 0.004% to 0.007% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL. In some embodiments, the final concentration of NaCl or KCl added to a supernatant that is effective to inhibit precipitation of residual host cell DNA by DB which had been added to the crude lysate at a final concentration of about 0.007% to 0.020% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL. In some embodiments, the final concentration of NaCl or KCl added to a supernatant that is effective to inhibit precipitation of residual host cell DNA by DB which had been added to the crude lysate at a final concentration of about 0.008% to 0.013% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL. In some embodiments, the final concentration of NaCl or KCl added to a supernatant that is effective to inhibit precipitation of residual host cell DNA by DB which had been added to the crude lysate at a final concentration of about 0.010% to 0.030% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL. In some embodiments, the final concentration of NaCl or KCl added to a supernatant that is effective to inhibit precipitation of residual host cell DNA by DB which had been added to the crude lysate at a final concentration of about 0.012% to 0.020% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL. In some embodiments, the final concentration of NaCl or KCl added to a supernatant that is effective to inhibit precipitation of residual host cell DNA by DB which had been added to the crude lysate at a final concentration of about 0.013% to 0.040% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL.

In some embodiments, the final concentration of NaCl or KCl added to a supernatant that is effective to inhibit precipitation of residual host cell DNA by DB which had been added to the crude lysate at a final concentration of about 0.016% to 0.027% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL. In some embodiments, the final concentration of NaCl or KCl added to a supernatant that is effective to inhibit precipitation of residual host cell DNA by DB which had been added to the crude lysate at a final concentration of about 0.017% to 0.050% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL. In some embodiments, the final concentration of NaCl or KCl added to a supernatant that is effective to inhibit precipitation of residual host cell DNA by DB which had been added to the crude lysate at a final concentration of about 0.020% to 0.033% per 1×10⁶ vc/mL is about 6.7 mM to 20.0 mM per 1×10⁶ vc/mL, 8.0 mM to 13.3 mM per 1×10⁶ vc/mL, 10.0 mM to 30.0 mM per 1×10⁶ vc/mL, 12.0 mM to 20.0 mM per 1×10⁶ vc/mL, 13.3 mM to 40.0 mM per 1×10⁶ vc/mL, 16.0 mM to 26.7 mM per 1×10⁶ vc/mL, 16.7 mM to 50.0 mM per 1×10⁶ vc/mL, 20.0 mM to 33.3 mM per 1×10⁶ vc/mL, 20.0 mM to 60.0 mM per 1×10⁶ vc/mL, 23.3 mM to 70.0 mM per 1×10⁶ vc/mL, 24.0 mM to 40.0 mM per 1×10⁶ vc/mL, 26.7 mM to 80.0 mM per 1×10⁶ vc/mL, 28.0 mM to 46.7 mM per 1×10⁶ vc/mL, or about 32.0 mM to 53.3 mM per 1×10⁶ vc/mL.

In some embodiments, the ratio of the final concentration of the added salt in the supernatant expressed in moles per liter (M) relative to the final concentration of domiphen halide, such as DB, added to precipitate host cell DNA from the crude host cell lysate expressed as % w/v or % w/w is at least or about 0.1, 0.2, 0.3, 0.4, 0.5, 0.6, 0.7, 0.8, 0.9, 1.0, 1.1, 1.2, 1.25, 1.3, 1.33, 1.4, 1.5, 1.6, 1.7, 1.8, 1.9, 2.0, 2.1, 2.2, 2.3, 2.4, 2.5, 2.6, 2.7, 2.8, 2.9, or 3.0, or a range including and between any two of the foregoing values, such as from about 3.0 to 0.1, 2.9 to 0.2, 2.8 to 0.2, 2.8 to 0.3, 2.7 to 0.2, 2.7 to 0.3, 2.7 to 0.4, 2.6 to 0.2, 2.6 to 0.3, 2.6 to 0.4, 2.6 to 0.5, 2.5 to 0.2, 2.5 to 0.3, 2.5 to 0.4, 2.5 to 0.5, 2.5 to 0.6, 2.5 to 0.7, 2.5 to 0.8, 2.5 to 0.9, 2.5 to 1.0, 2.5 to 1.1, 2.5 to 1.2, 2.5 to 1.3, 2.5 to 1.4, 2.5 to 1.5, 2.5 to 1.6, 2.5 to 1.7, 2.5 to 1.8, 2.5 to 1.9, 2.5 to 2.0, 2.5 to 2.1, 2.5 to 2.2, 2.5 to 2.3, 2.5 to 2.4, 2.4 to 0.2, 2.4 to 0.3, 2.4 to 0.4, 2.4 to 0.5, 2.4 to 0.6, 2.4 to 0.7, 2.4 to 0.8, 2.4 to 0.9, 2.4 to 1.0, 2.4 to 1.1, 2.4 to 1.2, 2.4 to 1.3, 2.4 to 1.4, 2.4 to 1.5, 2.4 to 1.6, 2.4 to 1.7, 2.4 to 1.8, 2.4 to 1.9, 2.4 to 2.0, 2.4 to 2.1, 2.4 to 2.2, 2.4 to 2.3, 2.3 to 0.2, 2.3 to 0.3, 2.3 to 0.4, 2.3 to 0.5, 2.3 to 0.6, 2.3 to 0.7, 2.3 to 0.8, 2.3 to 0.9, 2.3 to 1.0, 2.3 to 1.1, 2.3 to 1.2, 2.3 to 1.3, 2.3 to 1.4, 2.3 to 1.5, 2.3 to 1.6, 2.3 to 1.7, 2.3 to 1.8, 2.3 to 1.9, 2.3 to 2.0, 2.3 to 2.1, 2.3 to 2.2, 2.2 to 0.2, 2.2 to 0.3, 2.2 to 0.4, 2.2 to 0.5, 2.2 to 0.6, 2.2 to 0.7, 2.2 to 0.8, 2.2 to 0.9, 2.2 to 1.0, 2.2 to 1.1, 2.2 to 1.2, 2.2 to 1.3, 2.2 to 1.4, 2.2 to 1.5, 2.2 to 1.6, 2.2 to 1.7, 2.2 to 1.8, 2.2 to 1.9, 2.2 to 2.0, 2.2 to 2.1, 2.1 to 0.2, 2.1 to 0.3, 2.1 to 0.4, 2.1 to 0.5, 2.1 to 0.6, 2.1 to 0.7, 2.1 to 0.8, 2.1 to 0.9, 2.1 to 1.0, 2.1 to 1.1, 2.1 to 1.2, 2.1 to 1.3, 2.1 to 1.4, 2.1 to 1.5, 2.1 to 1.6, 2.1 to 1.7, 2.1 to 1.8, 2.1 to 1.9, 2.1 to 2.0, 2.0 to 0.2, 2.0 to 0.3, 2.0 to 0.4, 2.0 to 0.5, 2.0 to 0.6, 2.0 to 0.7, 2.0 to 0.8, 2.0 to 0.9, 2.0 to 1.0, 2.0 to 1.1, 2.0 to 1.2, 2.0 to 1.3, 2.0 to 1.4, 2.0 to 1.5, 2.0 to 1.6, 2.0 to 1.7, 2.0 to 1.8, 2.0 to 1.9, 1.9 to 0.2, 1.9 to 0.3, 1.9 to 0.4, 1.9 to 0.5, 1.9 to 0.6, 1.9 to 0.7, 1.9 to 0.8, 1.9 to 0.9, 1.9 to 1.0, 1.9 to 1.1, 1.9 to 1.2, 1.9 to 1.3, 1.9 to 1.4, 1.9 to 1.5, 1.9 to 1.6, 1.9 to 1.7, 1.9 to 1.8, 1.8 to 0.2, 1.8 to 0.3, 1.8 to 0.4, 1.8 to 0.5, 1.8 to 0.6, 1.8 to 0.7, 1.8 to 0.8, 1.8 to 0.9, 1.8 to 1.0, 1.8 to 1.1, 1.8 to 1.2, 1.8 to 1.3, 1.8 to 1.4, 1.8 to 1.5, 1.8 to 1.6, 1.8 to 1.7, 1.7 to 0.2, 1.7 to 0.3, 1.7 to 0.4, 1.7 to 0.5, 1.7 to 0.6, 1.7 to 0.7, 1.7 to 0.8, 1.7 to 0.9, 1.7 to 1.0, 1.7 to 1.1, 1.7 to 1.2, 1.7 to 1.3, 1.7 to 1.4, 1.7 to 1.5, 1.7 to 1.6, 1.6 to 0.2, 1.6 to 0.3, 1.6 to 0.4, 1.6 to 0.5, 1.6 to 0.6, 1.6 to 0.7, 1.6 to 0.8, 1.6 to 0.9, 1.6 to 1.0, 1.6 to 1.1, 1.6 to 1.2, 1.6 to 1.3, 1.6 to 1.4, 1.6 to 1.5, 1.5 to 0.2, 1.5 to 0.3, 1.5 to 0.4, 1.5 to 0.5, 1.5 to 0.6, 1.5 to 0.7, 1.5 to 0.8, 1.5 to 0.9, 1.5 to 1.0, 1.5 to 1.1, 1.5 to 1.2, 1.5 to 1.3, 1.5 to 1.4, 1.4 to 0.2, 1.4 to 0.3, 1.4 to 0.4, 1.4 to 0.5, 1.4 to 0.6, 1.4 to 0.7, 1.4 to 0.8, 1.4 to 0.9, 1.4 to 1.0, 1.4 to 1.1, 1.4 to 1.2, 1.4 to 1.3, 1.3 to 0.2, 1.3 to 0.3, 1.3 to 0.4, 1.3 to 0.5, 1.3 to 0.6, 1.3 to 0.7, 1.3 to 0.8, 1.3 to 0.9, 1.3 to 1.0, 1.3 to 1.1, 1.3 to 1.2, 1.2 to 0.2, 1.2 to 0.3, 1.2 to 0.4, 1.2 to 0.5, 1.2 to 0.6, 1.2 to 0.7, 1.2 to 0.8, 1.2 to 0.9, 1.2 to 1.0, 1.2 to 1.1, 1.1 to 0.2, 1.1 to 0.3, 1.1 to 0.4, 1.1 to 0.5, 1.1 to 0.6, 1.1 to 0.7, 1.1 to 0.8, 1.1 to 0.9, 1.1 to 1.0, 1.0 to 0.2, 1.0 to 0.3, 1.0 to 0.4, 1.0 to 0.5, 1.0 to 0.6, 1.0 to 0.7, 1.0 to 0.8, 1.0 to 0.9, 0.9 to 0.2, 0.9 to 0.3, 0.9 to 0.4, 0.9 to 0.5, 0.9 to 0.6, 0.9 to 0.7, 0.9 to 0.8, 0.8 to 0.2, 0.8 to 0.3, 0.8 to 0.4, 0.8 to 0.5, 0.8 to 0.6, 0.8 to 0.7, 0.7 to 0.2, 0.7 to 0.3, 0.7 to 0.4, 0.7 to 0.5, 0.7 to 0.6, 0.6 to 0.2, 0.6 to 0.3, 0.6 to 0.4, 0.6 to 0.5, 0.5 to 0.2, 0.5 to 0.3, 0.5 to 0.4, 0.4 to 0.2, 0.4 to 0.3, or 0.3 to 0.2.

Addition of a salt to the supernatant raises the ionic strength of the supernatant, which can be expressed in various ways, such as an increase in the conductivity of the solution, which can be measured using a conductivity probe and meter, use of which is within the knowledge of those of ordinary skill, and can be expressed in suitable units such as milliSiemens per centimeter (mS/cm). In some embodiments, addition of a salt to the supernatant increases the conductivity of the supernatant relative to its conductivity before addition of a salt, by at least or about 5 mS/cm, 10 mS/cm, 15 mS/cm, 20 mS/cm, 25 mS/cm, 30 mS/cm, 35 mS/cm, 40 mS/cm, 45 mS/cm, 50 mS/cm, 55 mS/cm, 60 mS/cm, 65 mS/cm, 70 mS/cm, 75 mS/cm, 80 mS/cm, 85 mS/cm, 90 mS/cm, 95 mS/cm, or 100 mS/cm, or a range of conductivity including and between any two of the foregoing values, such as 5 mS/cm to 60 mS/cm, 10 mS/cm to 60 mS/cm, 15 mS/cm to 60 mS/cm, 15 mS/cm to 55 mS/cm, 15 mS/cm to 50 mS/cm, 15 mS/cm to 45 mS/cm, 15 mS/cm to 40 mS/cm, or 15 mS/cm to 35 mS/cm, or some other range.

In some embodiments, after mixing, the mixture of the supernatant and salt solution can be filtered, for example by depth filtration, ultrafiltration, nanofiltration, or diafiltration. In some embodiments, filtration is performed using one or more membrane filters, such as a membrane filter with an average pore size of less than or equal to about 10 μm, 5 μm, 2 μm, 1 μm, 0.5 μm, 0.2 μm, or 0.1 μm.

In some embodiments, after mixing and optionally filtering, the mixture of the supernatant and salt solution can be held (incubated), such as in storage in a suitable container of some kind, such as a bag, break tank, or single use mixer, or other container known in the art, for a period of time without active mixing before performing at least one additional downstream processing step intended to purify a biological product of the host cells in the mixture. In some embodiments, the holding period is less than or equal to about 96 hrs, 72 hrs, 48 hrs, 36 hrs, 24 hrs, 12 hrs, 9 hrs, 6 hrs, 3 hrs, 2 hrs, 90 mins, 60 mins, 45 mins, 30 mins, 15 mins, or 10 mins. In some embodiments, the mixture is held at about room temperature, for example, about 20° C. to 22° C., or some other temperature, such as about 2° C. to 8° C., 4° C., or 37° C.

As described in the Examples section, turbidity due to precipitation of residual host cell DNA by DB can increase with time and the resulting precipitate can dramatically reduce the efficiency of downstream processing steps, such as affinity chromatography, and addition of a salt, such as NaCl, KCl or others, can inhibit this ongoing precipitation in a concentration dependent manner. Furthermore, the inhibitory effect of higher salt concentrations is most apparent over longer periods of time during which precipitation might occur. Consequently, in some embodiments, it may be possible to use lower final concentrations of added salt to inhibit ongoing precipitation of residual host cell DNA if a hold time is relatively short, and conversely, higher final concentrations of added salt to inhibit ongoing precipitation if a hold time is comparatively long. For example, in some embodiments, a salt, such as NaCl or KCl, can be added to the supernatant to a final concentration of about 100 mM, or from about 100 mM to 200 mM, 100 mM to 300 mM, 100 mM to 400 mM, 100 mM to 500 mM, 100 mM to 600 mM, if the hold time between salt addition and a subsequent purification step, such as chromatography, is about 24 hours or less time. In other embodiments, a salt, such as NaCl or KCl, can be added to the supernatant to a final concentration of about 200 mM, or from about 200 mM to 300 mM, 200 mM to 400 mM, 200 mM to 500 mM, 200 mM to 600 mM, or about 300 mM, or from about 300 mM to 400 mM, 300 mM to 500 mM, or 300 mM to 600 mM, or about 400 mM, or from about 400 mM to 500 mM, or 400 mM to 600 mM if the hold time between salt addition and a subsequent purification step, such as chromatography, is about 36 hours or less time. In some other embodiments, a salt, such as NaCl or KCl, can be added to the supernatant to a final concentration of about 300 mM, or from about 300 mM to 400 mM, 300 mM to 500 mM, or 300 mM to 600 mM, or about 400 mM, or from about 400 mM to 500 mM, or 400 mM to 600 mM if the hold time between salt addition and a subsequent purification step, such as chromatography, is about 48 hours or less time. And in yet other embodiments, a salt, such as NaCl or KCl, can be added to the supernatant to a final concentration of about 400 mM, or from about 400 mM to 500 mM, or 400 mM to 600 mM, or about 500 mM, or from about 500 mM to 600 mM, or about 600 mM if the hold time between salt addition and a subsequent purification step, such as chromatography, is about 72 hours or less time.

In any of the embodiments described above, turbidity of a supernatant (i.e., clarified host cell lysate) caused by precipitation of residual host cell DNA can be monitored at any convenient stage or time, such as before or shortly after addition and mixing with a salt solution and optionally subsequent filtration, as well as at one or more subsequent times, such as during a period of holding or storage of the supernatant but before, in some embodiments, it is processed according to one or more additional steps directed to further purifying the desired biological product, such as an AAV vector. Turbidity can be detected and quantified in any way that is known in the art, such as by using an electronic turbidity meter (turbidimeter), nephelometer, spectrophotometer, or the like, and expressed in any convenient units, such as nephelometric turbidity units (NTU) or Jackson turbidity units (JTU). See, e.g., Zhu, Y, et al., Development of a New Method for Turbidity Measurement Using Two NIR Digital Cameras, ACS Omega 5:5421-8 (2020); Bin Omar, F A and M Z Bin MatJafri, Turbidimeter Design and Analysis: A Review on Optical Fiber Sensors for the Measurement of Water Turbidity, Sensors 9:8311-35 (2009).

In some embodiments adding salt to the supernatant prepared from detergent lysed host cells is effective to inhibit precipitation of residual host cell DNA by DB so that the turbidity of the supernatant is less than or about 100 NTUs, 50 NTUs, 40 NTUs, 30 NTUs, 20 NTUs, 15 NTUs, 14 NTUs, 13 NTUs, 12 NTUs, 11 NTUs, 10 NTUs, 9 NTUs, 8 NTUs, 7 NTUs, 6 NTUs, 5 NTUs, 4 NTUs, 3 NTUs, 2 NTUs, 1 NTU, or less, or a range of including and between any two of the foregoing values. In some embodiments, turbidity is measured shortly after addition and mixing with a salt solution and optionally subsequent filtration, such as within an hour or less time, or some period of time after salt addition, such as during or after a holding period of less than or equal to about 96 hrs, 72 hrs, 48 hrs, 36 hrs, 24 hrs, 12 hrs, 9 hrs, 6 hrs, 3 hrs, 2 hrs, 90 mins, 60 mins, 45 mins, 30 mins, 15 mins, or 10 mins, or some range of time including and between any two of the foregoing values.

Systems for Performing Methods

In some embodiments, the salt solution is added to the supernatant in a batch as one or more boluses, and mixed with the supernatant during and/or after the addition of the salt solution. Such mixing can be performed in a containment vessel of any suitable size and configuration, such as a bottle, tank, stirred-tank bioreactor, or the like. In this manner, the entire volumes of the supernatant and salt solution are combined with each other over the course of one or more discrete steps. In such embodiments, mixing can be performed in any way known the art to be effective for mixing solutions, such as by using impellers or pumping.

In other embodiments, the bulk of the supernatant and salt solution can be maintained separately while being mixed together in a continuous process. In some embodiments, a continuous mixing process can be effected using a system comprising one or more container means separately containing the supernatant and the salt solution, a mixing means, and means for fluid communication from the respective container means of the separate solutions to the mixing means. In some embodiments, the system further comprises means for fluid communication from the mixing means to means downstream for variously storing the mixture, filtering the mixture, and/or further purifying a biological product from the mixture. In some embodiments, the system further comprises means for actively moving fluid from the several containment means to the mixing means, such as a pump means, for example, a peristaltic pump, and the like. Systems of the disclosure can comprise a single pump means or a plurality thereof. In some embodiments, systems of the disclosure further comprise means for filtering the supernatant before it is mixed with the salt solution, and/or filtering the mixture of the supernatant and the salt solution after mixing.

In some embodiments, a container means can be any type of container known the art to be suitable for holding a solution comprising a biological product, examples of which are bottles, tanks, carboys, plastic bags, or bioreactors. Fluid communication between containment means and a mixing means downstream can be effected in any way known in the art for continuously conveying solutions from one place to another, examples being pipes or tubes. Such fluid communication means can be a separate work piece temporarily brought into contact with the solution through an opening in the container means (such as a tube placed into a tank from an opening at its top), or affixed permanently or semi-permanently in some fashion to an attachment point of the containment means (e.g., by clamping, gluing, or via a friction joint), or can be an integral with the containment means (e.g., by welding). Mixing means can be any device or mechanism known the art to be effective for mixing together solutions, at least one containing a biological product, such as impellers of various configuration, stir bars, rotor/stator combinations, or static in-line mixers. Containment means, fluid communication means and mixing means can each be made of any material known in the art to be compatible with biological products, such as stainless steel, glass, and plastics, such as polyethylene, or a combination of such materials. Fluid communication means can comprise a single means or plurality of means for effecting fluid communication. For example, a system of the disclosure can comprise a plurality of tubes that attach to and fluidly connect other components of the system, such as container means, filter means, and mixing means.

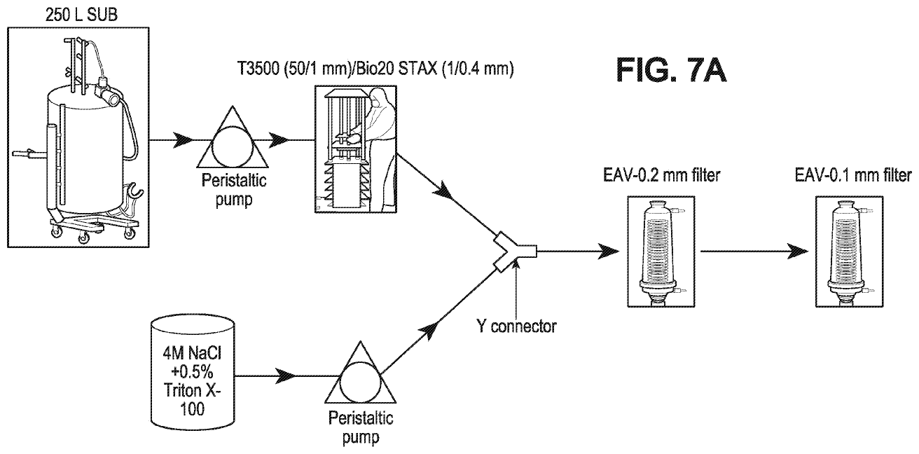

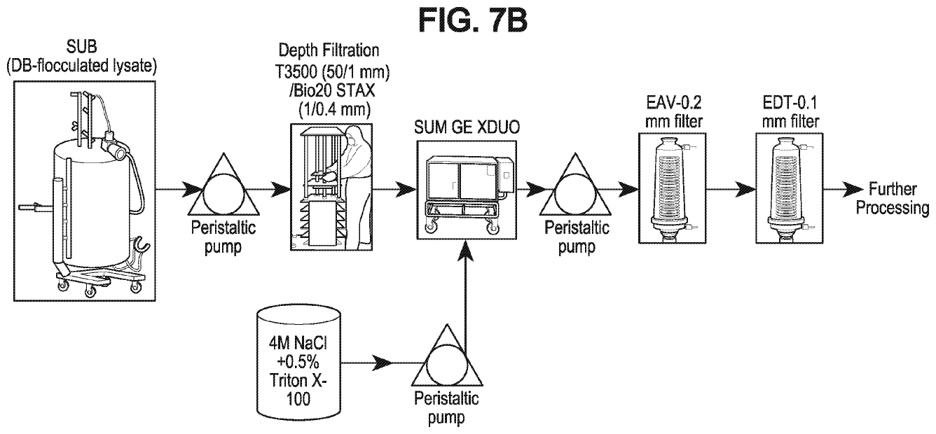

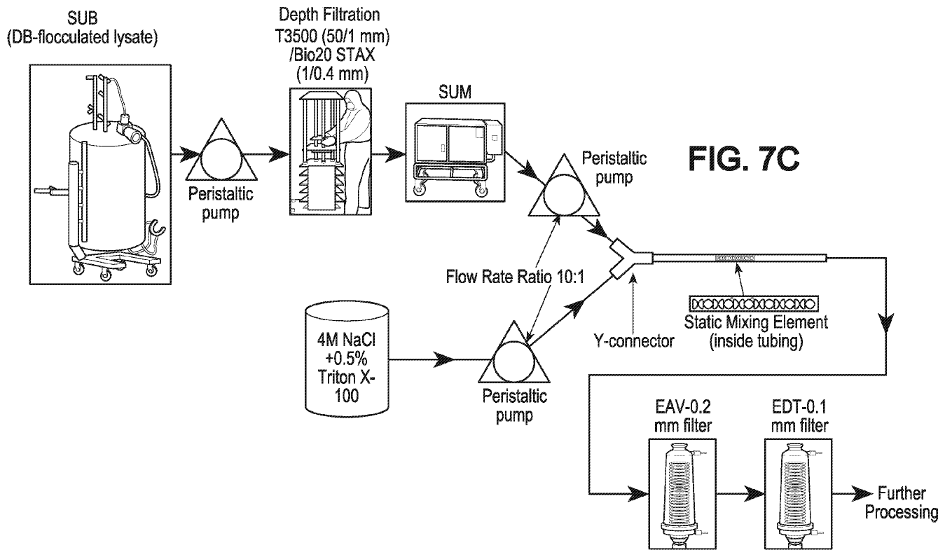

A non-limiting example of a system for performing methods of the disclosure is illustrated in FIG. 7A. In this system supernatant is held temporarily in a single use bioreactor (SUB) and a concentrated solution comprising a salt (here NaCl) is held in a separate container. A tube runs from the SUB and container for the salt solution allowing fluid to exit from each. Each tube is loaded into peristaltic pump so that each solution can be pumped out of its respective container at a defined rate, which can be the same or different. The tubes then meet at a hollow T connector or Y connector where the two solutions can mix together and then travel as a mixture downstream. As depicted, the supernatant can be filtered before being mixing, such as by positioning a depth filtration apparatus downstream pump for the supernatant and upstream of the hollow connector, through which the supernatant can pass and be filtered before it progresses via an outlet tube to the hollow connector for mixing. Also as depicted, after mixing, the mixture can be filtered, such as by positioning one or more membrane filters downstream of the hollow connector, and connected thereto by other tubing.

Another non-limiting example of a system for performing methods of the disclosure is illustrated in FIG. 7B. In this system, supernatant and salt solution are temporarily held, pumped and filtered as in the first system but instead of combining and mixing at a hollow connector, are instead pumped into a single use mixer (SUM), such as an Xcellerex XDUO SUM (Cytiva), in which supernatant and salt solution are mixed together. Thereafter, a peristaltic pump pumps the mixture out of the SUM and optionally through membrane filters via connecting tubing. This system can be operated batchwise, meaning the bulk of supernatant and salt solution can be pumped from their containers into the SUM where they are mixed, and thereafter pumped out of the SUM for downstream processing. Alternatively, this system can be operated continuously, meaning mixture formed in the SUM can be pumped out for downstream processing at the same time that supernatant and salt solution are pumped in for mixing.

Yet another non-limiting example of a system for performing methods of the disclosure is illustrated in FIG. 7C. In this system, supernatant and salt solution are temporarily held, pumped and filtered as in the first system, but mixing efficiency is enhanced by pumping the two solutions through a static in-line mixing element, of which numerous configurations are possible, positioned downstream of a hollow T or Y connector where supernatant and salt solution initially combine. Use of such static in-line mixing elements may improve mixing in various circumstances, such as when the supernatant and the salt solution have significantly different viscosities, which may occur when the salt solution contains a high concentration of salt, making it more viscous. A system can further include a break tank, here depicted as an SUM, allowing faster pumping of the supernatant if desired (for example, to maintain sufficient pressure for effective depth filtering), positioned downstream of the depth filter and upstream of the mixer, as well as an additional pump to pump supernatant from the break tank to the mixer. Also as depicted, after mixing, the mixture can be filtered, such as by positioning one or more membrane filters downstream of the mixer, and connected thereto by other tubing.

As noted above, pumping flow rates for the supernatant and salt solution can be the same or different for purposes of controlling the relative amount of salt solution to be mixed with supernatant to achieve a desired final salt concentration in the mixture. For example, if the desired final concentration of added salt in the mixture is to be about 400 mM, a stock solution comprising 4 M salt can be prepared. Thereafter, the supernatant and concentrated salt solution can be pumped at a relative rate of 10:1 to achieve the desired final salt concentration. In some embodiments, the conductance of the mixture can be monitored in real time with feedback control used to adjust the relative pump rate to bring the conductance back into an acceptable range. Any suitable relative pump rates are possible in view of the desired final concentration of salt in the mixture and its concentration in the stock solution.

Downstream Purification Steps

As used herein, the terms “purify,” “purified,” “purification,” and the like, when used in connection with a biological product, or sample or preparation thereof, indicate a relative increase or improvement in purity compared with a starting material from which the biological product is derived, and/or a prior intermediate purification step in some scheme of sequential purification steps intended to purify the biological product, and does not require a particular qualitative or quantitative degree of purity, unless otherwise specified.

In some embodiments, after mixing the supernatant and salt solution, and optionally filtering and/or holding the mixture, a biological product in the mixture can be further purified in at least one downstream processing step known in the art to be effective to purify such biological product. For example, if the product is a monoclonal antibody, then the product could be purified by pumping the mixture through an affinity chromatography column in which the resin or matrix used to fill the column contains protein A. In other embodiments, depending on the nature of the biological product, the downstream processing step can comprise precipitation in a lyotropic salt, such as ammonium sulfate. In other embodiments the downstream processing step can comprise performing at least type of chromatography. Many types of chromatography useful in the methods of the disclosure are known in the art including, without limitation, size exclusion chromatography (SEC); affinity chromatography, using any affinity ligand attached to the chromatography resin or matrix capable of specific binding to the biological product, such as an antibody, or antigen binding fragment thereof, lectin, protein A, protein G, protein L, or glycan, etc.; immobilized metal chelate chromatography (IMAC); thiophilic adsorption chromatography; hydrophobic interaction chromatography (HIC); multimodal chromatography (MMC); pseudoaffinity chromatography; and ion exchange chromatography (IEX or IEC), such as anion exchange chromatography (AEX) or cation exchange chromatography (CEX). In other embodiments, the downstream processing step can comprise desalting or buffer exchange, filtering, such as ultrafiltration, nanofiltration, and/or diafiltration, or concentrating the biological product, for example using tangential flow filtration (TFF). Use of more than one downstream processing step is possible, and the plurality of downstream processing steps can be performed in any order according to the knowledge of those ordinarily skilled in the art.

In some embodiments, the biological product is an AAV vector, and the downstream step useful for further purifying the vector can comprise, without limitation, performing at least one chromatography step. In some embodiments, the chromatography step comprises antibody-based affinity ligand purification in which an antibody, or antibody fragment thereof, is attached to a stationary phase matrix or resin loaded into a chromatography column which is then equilibrated with a suitable buffer, followed by pumping the supernatant and salt solution mixture containing the vector through the column, and then eluting the vector that specifically bound to the ligand. In some embodiments, the antibody bound to the solid phase can be an IgG, or fragment thereof, or a single-chain camelid antibody (such as a heavy chain variable region camelid antibody). Non-limiting examples of such resins include Sepharose AVB, POROS CaptureSelect AAVX, POROS CaptureSelect AAV8, and POROS CaptureSelect AAV9. See, e.g., Terova, O, et al., Affinity Chromatography Accelerates Viral Vector Purification for Gene Therapies, BioPharm Intl. eBook pp. 27-35 (2017); Mietzsch, M, et al., Characterization of AAV-Specific Affinity Ligands: Consequences for Vector Purification and Development Strategies, Mol. Ther. Meth. & Clin. Dev., 19:362-73 (2020); Rieser, R, et al., Comparison of Different Liquid Chromatography-Based Purification Strategies for Adeno-Associated Virus Vectors, Pharmaceutics 13, 748 (2021) (doi.org/10.3390/pharmaceutics13050748).

In other embodiments, the chromatography step comprises use of a stationary phase to which is bound the same type of ligand that certain AAV serotypes use in binding to cells, such as a glycan, such as sialic acid (e.g., an O-linked or N-linked sialic acid), galactose, heparin, or heparan sulfate, or a proteoglycan, such as a heparan or heparin sulfate proteoglycan (HSPG). For example, an affinity matrix containing sialic acid residues can be used to purify AAV vectors with capsids that specifically bind to sialic acid (e.g., AAV1, AAV4, AAV5, or AAV6); an affinity matrix containing galactose can be used to purify AAV vectors with capsids that specifically bind to galactose (e.g., AAV9); and an affinity matrix containing heparin, heparan, or HSPG can be used to purify AAV vectors with capsids that specifically bind to HSPG (e.g., AAV2, AAV3, AAV3b, AAV6, or AAV13). In yet other exemplary non-limiting embodiments, depending on the physicochemical characteristics of the vector, such as the charge on the capsid, AAV vectors can be further purified by performing anion exchange, cation exchange, or hydrophobic interaction chromatography. Any other downstream process step useful for purifying AAV vectors known in the art may be used as well.

In some embodiments, the methods of the disclosure are effective to improve the performance of at least one downstream processing step. In some embodiments, the downstream processing step is affinity chromatography and improved performance is measured as a number of purification cycles before yield of a biological product, for example, an AAV vector, falls below a certain threshold, such as less than 80%, 70%, 60%, or 50%. As known in the art, chromatography is typically performed by packing a chromatography column of any suitable size with fresh, unused stationary phase resin or matrix suitable for the type of chromatography being performed, such as an affinity resin, and then washing and/or equilibrating the resin with suitable equilibration solution(s) in preparation for loading the column with the sample to be purified. The column is then ready for the first purification cycle in which a liquid sample containing the biological product to be purified is loaded onto and pumped through the column. Once the entire sample has been pumped through, the column may be washed with any suitable non-denaturing wash solution(s) to remove contaminants while the biological product is retained, usually non-covalently, on the stationary phase. The product can thereafter be eluted by pumping through the column any suitable elution solution and collecting the eluate, which may be collected in elution fractions which are thereafter tested to determine the amount of biological product in each, after which fractions containing a significant amount of the biological product can be pooled. In some embodiments, after elution, the stationary phase can be cleaned in place with any suitable clean in place solution(s) containing chemicals, such as acids, bases or chaotropic salts, to remove any residual product or contaminants, and then re-equilibrated with the equilibration solution in preparation for a subsequent purification cycle. Thus, as used herein, a purification cycle comprises running a sample through a chromatography column and then eluting the desired biological product, such as an AAV vector, retained on the stationary phase.

In the context of a downstream process step involving chromatography, “yield” of a biological product means the total amount of the product in the eluate pool expressed as a percentage of the total amount of the product in a sample before the chromatography step. The amount of the biological product can be determined using any method known in the art. For example, if the product is an AAV vector, the amount of the vector can be quantified using quantitative PCR (pPCR) using primers against the ITRs, or sequences in the transgene or other parts of the expression cassette, or using digital droplet PCR (ddPCR), and expressed as a titer in terms of vector genomes per unit volume, such as milliliters (vg/mL). See, e.g., Dobnik, D, et al., Accurate Quantification and Characterization of Adeno-Associated Viral Vectors, Front. Microbiol., Vol. 10, Art. 1570, pp. 1-13 (2019); Wang, Y, et al., A qPCR Method for AAV Genome Titer with ddPCR-Level of Accuracy and Precision, Mol. Ther.: Methods & Clin. Devel., 19:341-6 (2020); Werling, N J, et al., Systematic Comparison and Validation of Quantitative Real-Time PCR Methods for the Quantitation of Adeno-Associated Viral Product, Hum. Gene Ther. Meth. 26:82-92 (2015).

In some embodiments, the methods of the disclosure are effective to purify an AAV vector by performing affinity chromatography, where the number of affinity chromatography purification cycles that can be performed before yield of an AAV vector falls below 80%, 70%, 60%, or 50% is at least 5, 6, 7, 8, 9, 10, 11, 12, 13, 14, 15, or more cycles. In some of these embodiments, the chromatography is immunoaffinity chromatography, and the vector contains a capsid that binds more strongly to sialic acid or galactose than to HSPG, or does not bind specifically, or only weakly binds to HSPG, for example, AAV1, AAV4, AAV5, or AAV9. In other embodiments, the methods of the disclosure are effective to purify an AAV vector by performing affinity chromatography, where the number of affinity chromatography purification cycles that can be performed before yield of an AAV vector falls below 50% is at least 5, 6, 7, 8, 9, 10, 11, 12, 13, 14, 15, or more cycles. In some of these embodiments, the chromatography is immunoaffinity chromatography, and the vector contains a capsid that binds more strongly to sialic acid or galactose than to HSPG, for example, AAV1, AAV4, AAV5, AAV6, or AAV9. In other embodiments, the methods of the disclosure are effective to purify an AAV vector by performing affinity chromatography, where the number of affinity chromatography purification cycles that can be performed before yield of an AAV vector falls below 60% is at least 5, 6, 7, 8, 9, 10, 11, 12, 13, 14, 15, or more cycles. In some of these embodiments, the chromatography is immunoaffinity chromatography, and the vector contains a capsid that binds more strongly to sialic acid or galactose than to HSPG, or does not specifically bind or weakly binds to HSPG, for example, AAV1, AAV4, AAV5, or AAV9. In other embodiments, the methods of the disclosure are effective to purify an AAV vector by performing affinity chromatography, where the number of affinity chromatography purification cycles that can be performed before yield of an AAV vector falls below 70% is at least 5, 6, 7, 8, 9, 10, 11, 12, 13, 14, 15, or more cycles. In some of these embodiments, the chromatography is immunoaffinity chromatography, and the vector contains a capsid that binds more strongly to sialic acid or galactose than to HSPG, or does not specifically bind or weakly binds to HSPG, for example, AAV1, AAV4, AAV5, or AAV9.

In some embodiments, the methods of the disclosure are effective to achieve a high yield and/or purity of an active ingredient, such as a desired biological product, such as an AAV vector, in drug substance or drug product. As used in this context, “yield” means the amount of an active ingredient in drug substance compared to the amount of the same compound or substance, or a precursor thereof, in a starting material used in the synthesis or production of the active ingredient. As used herein, “drug substance” means a preparation comprising a substantially purified active ingredient resulting from a complete process intended to purify the active ingredient where the process is complete if, as designed or used in practice, it does not include or require any further steps intended to remove contaminants or further purify the active ingredient. For clarity, such further steps would not include buffer exchange, volume reduction, or addition of excipients, or like, which are merely intended to prepare drug product from drug substance, where “drug product” is the finished dosage form of the active ingredient as it would be marketed or used for administration to patients. As used herein, an “active ingredient” is any compound or substance, including a biologically derived substance, intended to furnish pharmacological activity or other direct effect in the diagnosis, cure, mitigation, treatment, or prevention of disease, or to affect the structure or any function of the human body. Non-limiting examples of active ingredients include viruses, vaccines, and virally derived vectors, such as AAV vectors and lentiviral vectors, and the like.

Purity of AAV vectors in a sample or preparation of such vectors can be determined and expressed in a variety of ways known in the art. For example, vector preparations can be analyzed on denaturing polyacrylamide gels and silver stained to detect proportions of the different viral proteins, VP1, VP2, and VP3, relative to contaminating cellular proteins. Different techniques can also be used to detect the proportion of full compared to empty capsids, with a greater percentage of full capsids indicating higher purity. As used herein, a “full capsid” is one that is concluded to contain a vector genome, and an “empty capsid” is a one that is concluded to contain either no or little nucleic acid. For example, capsids in vector preparations can be visualized using transmission electron microscopy, including cryoEM, and the numbers of full and empty capsids counted manually or using computerized image recognition algorithms. Even greater resolution can be achieved using analytical ultracentrifugation, which can discriminate between full, partially full and empty capsids. A convenient method for estimating AAV vector purity in terms of amount of contaminating empty capsids is to measure the UV light absorbance of a vector preparation at 260 nm and 280 nm, deriving the A260/A280 ratio. By calculating the theoretical extinction coefficients for a particular vector's capsid and genome, the relative concentrations of its capsid and genome in a preparation can be calculated from the A260/A280 ratio, with higher A260/A280 values indicating a greater proportion of full capsids. Additional information about methods for testing vector purity are described in Burnham B, et al., Analytical ultracentrifugation as an approach to characterize recombinant adeno-associated viral vectors, Hum. Gene Ther. Meth., 26(6):228-242 (2015); Subramanian, S, et al., Filling Adeno-Associated Virus Capsids: Estimating Success by Cryo-Electron Microscopy, Hum. Gene Ther., 30(12):1449-60 (2019); McIntosh, N L, et al., Comprehensive characterization and quantification of adeno associated vectors by size exclusion chromatography and multi angle light scattering, Nat. Sci. Reports, 11:3012, pp. 1-12 (2021); Sommer, J M, et al., Quantification of Adeno-Associated Virus Particles and Empty Capsids by Optical Density Measurement, Mol. Ther., 7(1):122-8 (2003); Wu, D, et al., Rapid Characterization of AAV gene therapy vectors by Mass Photometry, bioRxiv 2021.02.18.431916 (doi.org/10.1101/2021.02.18.431916).

In some embodiments, the methods of the disclosure are effective to achieve an acceptably low burden of host cell DNA in drug substance or drug product containing a desired biological product, such as an AAV vector, produced by host cells. The amount of host cell DNA in a sample or preparation, such as a detergent lysate of such cells, or a composition comprising biological product (such as drug substance or drug product) purified from such cells, such as an AAV vector, can be determined in any way known in the art. For example, sensitive qPCR assays have been developed designed to specifically detect repetitive sequence elements unique to the human genome (e.g., Alu repeats), and that of other species whose host cells are commonly used in manufacturing. See, e.g., Zhang, W, et al., Development and qualification of a high sensitivity, high throughput Q-PCR assay for quantitation of residual host cell DNA in purification process intermediate and drug substance samples, J. Pharma Biomed Anal 100:145-9 (2014); Wang, Y, et al., A Digestion-free Method for Quantification of Residual Host Cell DNA in rAAV Gene Therapy Products, Mol. Ther. 13:526-31 (2019). The amount of host cell DNA can be quantified and expressed as an absolute amount, such as mass in picograms (pg) or nanograms (ng), etc., in a volume, such as milliliter, or other unit, such as dose. Amounts of host cell DNA can also be normalized relative to another variable, such as the amount of AAV vector in a sample, which in some embodiments can be quantified and expressed as the number of vector genomes per milliliter, dose, etc.

What constitutes an acceptably low burden of host cell DNA in drug substance or drug product will be apparent to those of ordinary skill in the art and may depend on the type of biological product in question, as well as expectations of industry, patients, and/or regulatory authorities, such as the US FDA or the EMA, which may change with time. See, e.g., Gombold, J, et al., Lot Release and Characterization Testing of Live-Virus-Based Vaccines and Gene Therapy Products, Part 2, Bioprocess Intl. 4:46-56 (2006); Wright, J F, Product-Related Impurities in Clinical-Grade Recombinant AAV Vectors: Characterization and Risk Assessment, Biomedicines 2(1):80-97 (2014); Wang, X, et al., Residual DNA Analysis in Biologics Development: Review of Measurement and Quantitation Technologies and Future Directions, Biotech Bioeng 109(2):307-17 (2012).

In some embodiments, the methods of the disclosure are effective to achieve a high yield and/or purity of an AAV vector and an acceptably low burden of host cell DNA in drug substance or drug product. In some embodiments, the yield of AAV vector produced using methods of the disclosure (including as well, in some embodiments, additional downstream purification steps) can be at least or about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, or 80% or more, or any percentage yield between and including any of the foregoing values. In some embodiments, the purity of AAV vector produced using methods of the disclosure (including as well, in some embodiments, additional downstream purification steps) can be expressed as the ratio of the UV absorbance measured at 260 nm and 280 nm (i.e., A₂₆₀/A₂₈₀) which, in some embodiments, can be at least or about 0.4, 0.5, 0.6, 0.7, 0.8, 0.9, 1.0, 1.1, 1.2, 1.3, 1.4, 1.5, 1.6, 1.7, or 1.8, or more, or an A₂₆₀/A₂₈₀ between and including any of the foregoing values. In some embodiments, the purity of AAV vector produced using methods of the disclosure (including as well, in some embodiments, additional downstream purification steps) can be expressed as the percentage of full capsids in a vector preparation which, in some embodiments, can be at least or about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or any percentage of full capsids between and including any of the foregoing values.

In some embodiments, an acceptably low burden of host cell DNA in drug substance or drug product is one that is less than or about 100 ng, 90 ng, 80 ng, 70 ng, 60 ng, 50 ng, 40 ng, 30 ng, 20 ng, 10 ng, 5 ng, 2 ng, or 1 ng per dose, or less, or any value between and including any of the foregoing values. In other embodiments, an acceptably low burden of host cell DNA in drug substance or drug product is one that is less than or about 50 ng, 40 ng, 30 ng, 20 ng, 10 ng, 5 ng, 2 ng, 1 ng, 0.9 ng, 0.8 ng, 0.7 ng, 0.6 ng, 0.5 ng, 0.4 ng, 0.3 ng, 0.2 ng, 0.1 ng, 0.09 ng, 0.08 ng, 0.07 ng, 0.06 ng, 0.05 ng, 0.04 ng, 0.03 ng, 0.02 ng, 0.01 ng, 0.009 ng, 0.008 ng, 0.007 ng, 0.006 ng, 0.005 ng, 0.004 ng, 0.003 ng, 0.002 ng, 0.001 ng per milliliter drug substance or drug product, or less, or any value between and including any of the foregoing values. In yet other embodiments, an acceptably low burden of host cell DNA in drug substance or drug product containing an AAV vector is one that is less than or about 1000, 500, 250, 200, 150, 100, 90, 80, 70, 60, 50, 45, 40, 35, 30, 25, 20, 15, 10, 5, 2, or 1 picograms per 1×10⁹ vector genomes (pg/1×10⁹ vg), or less, or any value between and including any of the foregoing values.

Other objects, features and advantages of the present invention will be apparent from the foregoing detailed description. It should be understood, however, that the detailed description and the specific examples that follow, while indicating specific embodiments of the invention, are given by way of illustration only, since various changes, modifications and equivalents within the spirit and scope of the invention will be apparent from the detailed description and examples to those of ordinary skill in the art, and fall within the scope of the appended claims.

Unless otherwise indicated, use of the term “or” in reference to one or more members of a set of embodiments is equivalent in meaning to “and/or,” and does not require that they be mutually exclusive of each other. Unless otherwise indicated, a plurality of expressly recited numeric ranges also describes a range the lower bound of which is derived from the lower or upper bound of any one of the expressly recited ranges, and the upper bound of which is derived from the lower or upper bound of any other of the expressly recited ranges. Thus, for example, the series of expressly recited ranges “10-20, 20-30, 30-40, 40-50, 100-150, 200-250, 275-300,” also describes the ranges 10-50, 50-100, 100-200, and 150-250, among many others. Unless otherwise indicated, use of the term “about” before a series of numerical values or ranges is intended to modify not only the value or range appearing immediately after it but also each and every value or range appearing thereafter in the same series. Thus, for example, the phrase “about 1, 2, or 3,” is equivalent to “about 1, about 2, or about 3.”

All publications and references, including but not limited to articles, abstracts, patents, patent applications (whether published or unpublished), and biological sequences (including, but not limited to those identified by specific database reference numbers) cited herein are hereby incorporated herein by reference in their entirety for all purposes to the same extent as if each individual publication or reference were specifically and individually indicated to be so incorporated by reference. Any patent application to which this application claims priority directly or indirectly is also incorporated herein by reference in its entirety.

Unless otherwise indicated, the examples below describe experiments that were or are performed using standard techniques well known and routine to those of ordinary skill in the art. The examples are illustrative, but do not limit the invention.

### EXAMPLES

### Example 1

### Concentration Dependent Precipitation of Host Cell DNA by Domiphen Bromide

A first series of experiments determined the effect of domiphen bromide (DB) concentrations on precipitation of host cell DNA (HC-DNA) and host cell protein (HCP) from detergent lysed host cells, and recovery of an adeno-associated viral (AAV) vector produced by the cells.

HEK293 cells were grown in suspension culture in a 50 L single use bioreactor (SUB) to a viable cell density of 4.9×10⁶ cells/mL and then transfected with three plasmids respectively containing AAV2 rep and AAV9 cap genes, adenoviral helper functions, and an expression cassette for producing a mini-dystrophin protein flanked by AAV2 ITRs (this exemplary vector is described further in WO 2017/221145). After incubation for 72 hours for AAV vector production, by which time the cells had reached a viable cell density of about 7.7×10⁶ cells/mL, 1000 mL samples of cell suspension were removed from the bioreactor and lysed by adding a 10% Triton X-100 stock solution (w/v) to a final Triton X-100 concentration of 0.5% (v/v) followed by agitation for 30 minutes at room temperature. A 10% DB stock solution (w/v) was then added to lysate samples to a final DB concentration ranging from 0.05% to 0.20% (v/v) and agitated for 30 minutes at room temperature to precipitate HC-DNA, which was allowed to settle out of solution for 30 minutes, also at room temperature. DB treated lysate samples were then centrifuged at 4000 RPM for 15 minutes to pellet the DNA, after which the supernatant (i.e., partially clarified lysate) was removed and reserved. To prevent precipitation of residual HC-DNA by DB (i.e., to quench precipitation), a 5 M NaCl stock solution was added to a final NaCl concentration of 0.25 M. The quenched supernatant was filtered through one or more 0.2 μm filters (EKV (Pall)) to produce the final clarified lysate. AAV vector was then purified from the samples by immunoaffinity chromatography specific for the AAV9 capsid in the vectors (8 mL of Poros CaptureSelect AAV9 in a 1 cm (d)×10 cm (h) column using an AKTA Avant 150 system).

The titer of AAV vector, and amounts of HCP and HC-DNA in the samples of final clarified lysate (starting material) and purified vector (chromatography column elutions) were tested. AAV vector titer was determined by quantitative polymerase chain reaction (qPCR) using primers against the transgene in the vector, and expressed as vector genomes per milliliter (VG/mL)); the amount of HCP was determined by ELISA and expressed as nanograms per mL (ng/mL); and the amount of HC-DNA was determined by qPCR and expressed as picograms per mL (pg/mL). To control for sample volume variability, the amounts of HCP and HC-DNA were normalized relative to the amount of vector. As shown in Table 1, the vector titer, and amounts of HCP and HC-DNA in the starting material were relatively consistent across all DB concentrations tested, whereas starting at 0.1% DB final concentration in the lysate, there was a concentration dependent reduction in HC-DNA demonstrating the effectiveness with which DB can precipitate HC-DNA from host cell detergent lysates.

TABLE 1

| | | Vector | Host Cell | Host Cell | Host Cell | Host Cell |
|---|---|---|---|---|---|---|
| | Vector Titer | Recovery | Protein | Protein | DNA | DNA |
| Sample Condition | (VG/mL) | Elution | (ng/mL) | (ng/VG) | (pg/mL) | (pg/VG) |
| 0.05% DB S/M | 4.43E+11 | | 185912.4187 | 4.20E−07 | 4172602.046 | 9.42E−06 |
| 0.05% DB Elution | 3.74E+13 | 60% | 10803.32454 | 2.89E−10 | 211181.319 | 5.65E−09 |
| 0.10% DB S/M | 3.77E+11 | | 186312.7268 | 4.94E−07 | 3686648.187 | 9.78E−06 |
| 0.10% DB Elution | 1.78E+13 | 72% | 2546.15416 | 1.43E−10 | 135064.6281 | 7.59E−09 |
| 0.15% DB S/M | 3.65E+11 | | 233679.9812 | 6.40E−07 | 67377.30411 | 1.85E−07 |
| 0.15% DB Elution | 3.75E+13 | 68% | 16766.41993 | 4.47E−10 | 242849.5903 | 6.48E−09 |
| 0.2% DB S/M | 3.05E+11 | | 191746.4728 | 6.29E−07 | 8917.267 | 2.92E−08 |
| 0.2% DB Elution | 3.21E+13 | 71% | 18642.66787 | 5.81E−10 | 173571.495 | 5.41E−09 |

A second series of experiments determined the effect of higher DB concentrations on precipitation of HC-DNA and host cell protein HCP from detergent lysed host cells, and recovery of an AAV vector produced by the cells.

Similar to that described above, HEK293 cells were grown in suspension culture and transfected to produce AAV vector in two bioreactors at 3.6 L volume. When the cells had reached a viable cell densities of about 1.4×10⁷ cells/mL and 1.7×10⁷ cells/mL, respectively, the cultures were combined and samples taken, including one of 3500 mL, and four of 200 mL. To each sample, Triton X-100 was added (0.5% final concentration) to lyse the cells, after which DB was added to precipitate HC-DNA. The final DB concentration in the 3500 mL sample as 0.2%, whereas the final DB concentrations in the four 200 mL samples ranged from 0.1% to 0.4%. Following DNA precipitation, all samples were filtered through a 1 μm depth filter (J700 (Pall)) and a 0.4 μm depth filter (Bio20 (Pall)). A solution of concentrated NaCl was then added to the filtered samples to different final concentrations ranging from 250 mM to 500 mM, followed by mixing for 30 mins at room temperature. After adding salt and mixing, the samples were filtered through 0.2 μm and 0.1 μm membrane filters (EAV and EDT, respectively (Pall)). On the same day (day 0), the samples were analyzed to quantify AAV vector titer, and amounts of HC-DNA and HCP, as described above. As shown in Table 2, the vector titer, and amounts of HCP and HC-DNA in the clarified lysates were comparable across all DB concentrations tested, whereas there was a concentration dependent reduction in HC-DNA demonstrating the effectiveness with which DB can precipitate HC-DNA in host cell detergent lysates at higher viable cell densities compared to those studied in the first experiments described in this example.

TABLE 2

| | | Host Cell | Host Cell | | |
|---|---|---|---|---|---|
| | Vector Titer | Protein | Protein | Host Cell DNA | Host Cell DNA |
| Sample Condition | (VG/mL) | (ng/mL) | (ng/VG) | (pg/mL) | (pg/VG) |
| 200 mL, post 0.5% Triton | 3.53E+12 | 445104.39 | 1.26271E−07 | 2947706.02 | 8.36229E−07 |
| (0.1% DB) | | | | | |
| 200 mL, post 0.1% DB, pre | 3.00E+12 | 438513.79 | 1.46171E−07 | 3721418.72 | 1.24047E−06 |
| flocculation settling | | | | | |
| 200 mL, post 0.1% DB, | 2.67E+12 | 472063.65 | 1.77135E−07 | 5041756.75 | 1.89184E−06 |
| post flocculation settling | | | | | |
| 200 mL 0.1% DB, post J700 | 2.51E+12 | 432594.14 | 1.72348E−07 | 4639292.23 | 1.84832E−06 |
| 200 mL 0.1% DB, post | 1.24E+12 | 250406.67 | 2.02349E−07 | 481445.89 | 3.89047E−07 |
| Bio20 | | | | | |
| 200 mL 0.1% DB, 0.25M | 1.21E+12 | 234586.31 | 1.93473E−07 | 840122.59 | 6.92885E−07 |
| NaCl, post EAV | | | | | |
| 200 mL, post 0.5% Triton | 3.52E+12 | 459569.48 | 1.30652E−07 | 4896184.62 | 1.39195E−06 |
| (0.2% DB) | | | | | |
| 200 mL, post 0.2% DB, pre | 4.84E+12 | 418035.09 | 8.63263E−08 | 595280.05 | 1.22928E−07 |
| flocculation settling | | | | | |
| 200 mL, post 0.2% DB, | 2.10E+12 | 401064.27 | 1.91211E−07 | 746076.35 | 3.55698E−07 |
| post flocculation settling | | | | | |
| 200 mL 0.2% DB, post J700 | 2.12E+12 | 389057.17 | 1.83734E−07 | 335859.2 | 1.58611E−07 |
| 200 mL 0.2% DB, post | 1.90E+12 | 421971.48 | 2.22676E−07 | 143200.53 | 7.55676E−08 |
| Bio20 | | | | | |
| 200 mL 0.2% DB, 0.25M | 1.89E+12 | 369643.03 | 1.95837E−07 | 137667.03 | 7.29362E−08 |
| NaCl, post EAV | | | | | |
| 200 mL 0.2% DB, 0.25M | 1.74E+12 | 366697.53 | 2.11049E−07 | 131352.63 | 7.55986E−08 |
| NaCl, post EDT | | | | | |
| 200 mL, post 0.3% DB, pre | 3.78E+12 | 391464.75 | 1.03699E−07 | 559565.87 | 1.48229E−07 |
| flocculation settling | | | | | |
| 200 mL, post 0.3% DB, | 1.99E+12 | 369953.54 | 1.86375E−07 | 15847.29 | 7.98352E−09 |
| post flocculation settling | | | | | |
| 200 mL 0.3% DB, post J700 | 2.00E+12 | 366680.83 | 1.8357E−07 | 19438.72 | 9.73152E−09 |
| 200 mL 0.3% DB, post | 1.81E+12 | 323113.55 | 1.7901E−07 | 17901.68 | 9.91783E−09 |
| Bio20 | | | | | |
| 200 mL 0.3% DB, 0.4M | 1.84E+12 | 331014.85 | 1.80144E−07 | 20055.39 | 1.09145E−08 |
| NaCl, post EAV | | | | | |
| 200 mL 0.3% DB, 0.4M | 1.69E+12 | 301536.29 | 1.78688E−07 | 20012.17 | 1.18591E−08 |
| NaCl, post EDT | | | | | |
| 200 mL, post 0.4% DB, pre | 4.59E+12 | 382219.99 | 8.3227E−08 | 1083153.64 | 2.35853E−07 |
| flocculation settling | | | | | |
| 200 mL, post 0.4% DB, | 1.97E+12 | 407982.27 | 2.07098E−07 | 10125.92 | 5.14006E−09 |
| post flocculation settling | | | | | |
| 200 mL 0.4% DB, post J700 | 1.69E+12 | 338323.92 | 2.00786E−07 | 6469.97 | 3.83974E−09 |
| 200 mL 0.4% DB, post | 1.68E+12 | 311574.64 | 1.85737E−07 | 6930.96 | 4.13172E−09 |
| Bio20 | | | | | |
| 200 mL 0.4% DB, 0.5M | 1.64E+12 | 337268.02 | 2.0628E−07 | 4523.2 | 2.76648E−09 |
| NaCl, post EAV | | | | | |
| 200 mL 0.4% DB, 0.5M | 1.57E+12 | 349968.22 | 2.22555E−07 | 4071.9 | 2.58944E−09 |
| NaCl, post EDT | | | | | |
| 3500 mL, post 0.5% Triton | 2.1525E+12 | 376497.58 | 1.74912E−07 | 5436338.86 | 2.52559E−06 |
| 3500 mL, post 0.2% DB, | 3.5325E+12 | 410765.41 | 1.16282E−07 | 1285951.82 | 3.64034E−07 |
| pre cell debris settling | | | | | |
| 3500 mL, post 0.2% DB, | 1.7925E+12 | 381034.17 | 2.12571E−07 | 478462.85 | 2.66925E−07 |
| post cell debris settling | | | | | |
| 3500 mL, post J700 | 9.495E+11 | 194576.49 | 2.04925E−07 | 30179 | 3.17841E−08 |
| 3500 mL, post Bio20 | 1.5225E+12 | 328094.29 | 2.15497E−07 | 21725.41 | 1.42696E−08 |
| 3500 mL, post EAV 0.2 um | 1.42E+12 | 359589.48 | 2.53232E−07 | 18250.59 | 1.28525E−08 |
| 3500 mL, post EDT 0.1 um | 1.3275E+12 | 337637.19 | 2.54341E−07 | 16349.73 | 1.23162E−08 |

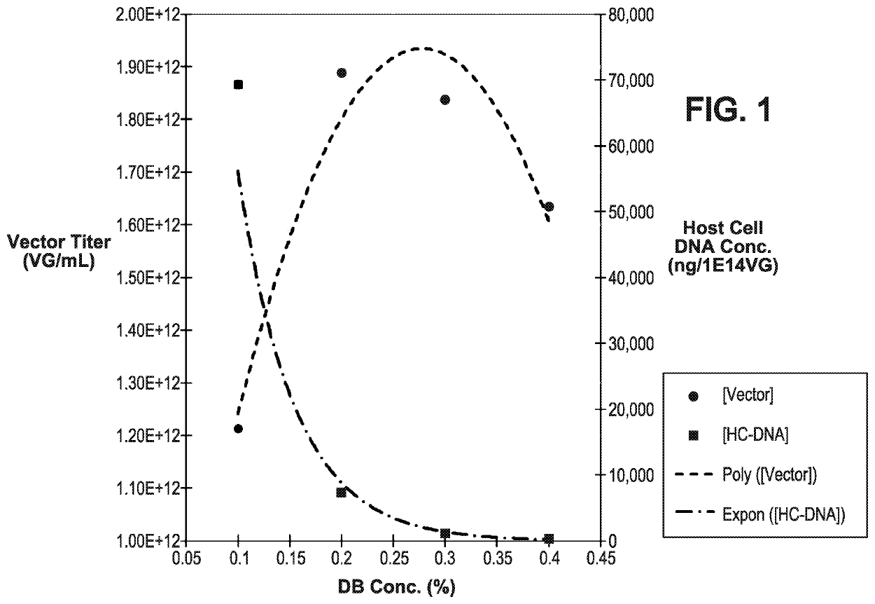

The concentration dependence of DB precipitation on vector titer and HC-DNA concentration in clarified HEK293 cell lysates is illustrated in FIG. 1, based on the data from measurements performed after the first membrane filtration step. In the graph, the vector titer curve is fitted to a polynomial equation, and the HC-DNA concentration is fitted to an exponential equation. The results indicate that under the conditions studied, DB concentration more than was required to effectively precipitate host cell DNA, and that DB concentrations above to 0.4% may start to reduce vector yield, possibly by sequestering vector in precipitated material.

Turbidity in the filtered lysates was also measured on day 0, and days 2 through 4 using a turbidimeter, and results expressed in nephelometric turbidity units (NTUs). As shown in Table 3, turbidity increased only modestly between day 0 and day 4 at all DB and NaCl concentrations tested.

TABLE 3

| Sample | DB final | NaCl final | NTUs | NTUs | NTUs | NTUs |
|---|---|---|---|---|---|---|
| Condition | conc. % | conc. mM | Day 0 | Day 2 | Day 3 | Day 4 |
| 3500 mL | 0.2 | 250 | 3.1 | 3.76 | 4.44 | 6.77 |
| 200 mL | 0.1 | 250 | 5.6 | 5.64 | 6.01 | 7.58 |
| 200 mL | 0.2 | 250 | 5.61 | 6.8 | 7.81 | 10.4 |
| 200 mL | 0.3 | 400 | 2.87 | 3.47 | 4.33 | 6.44 |
| 200 mL | 0.4 | 500 | 2.85 | 4.35 | 5.48 | 9.11 |

A third series of experiments examined the effect of varying the concentration of Triton X-100 used to lyse host cells and DB used to precipitate host cell DNA on AAV vector yield and the amount of HC-DNA in clarified lysate after DNA precipitation.

Similar to that described above, HEK293 cells were grown in suspension culture and transfected to produce AAV vector. After reaching a viable cell density of about 2×10⁷ cells/mL, mL samples were withdrawn and processed according to a matrix of final concentrations of Triton X-100 to lyse the cells, DB to precipitate host cell DNA in the crude lysates, and NaCl to inhibit precipitation by DB of residual host cell DNA in partially clarified lysates. Cells were mixed for 30 mins after Triton X-100 addition to produce cell lysate, to which DB was added and mixed for 30 mins to precipitate HC-DNA, followed by centrifugation to pellet the precipitated HC-DNA. Supernatants from the treated samples were reserved followed by salt addition and storage for two days. Vector titer and HC-DNA in the cell suspension and from the treated samples were quantified on day 0 and used to calculate the vector yield and the amount of residual host cell DNA in the samples relative to the starting cell suspension. Turbidity of the clarified lysates was measured on day 0 and again 47 hours later to measure precipitation by DB of residual host cell DNA in the clarified lysates. Experimental conditions and results are described in Table 4, including the final concentrations of Triton X-100, DB, and NaCl in the treated samples, vector titer and yield, and amount of HC-DNA expressed both as concentration and normalized to vector titer.

TABLE 4

| | Triton | | | | | | | | |
|---|---|---|---|---|---|---|---|---|---|
| | X-100 | DB | NaCl | | | | HC-DNA | | Turbidity |
| | Final | Final | Final | Vector | Vector | HC-DNA | Relative | Turbidity | After 47 |
| Sample | Conc. | Conc. | Conc. | Titer | Yield | Conc. | to Vector | Day 0 | Hrs |
| No. | (% w/w) | (% w/w) | (mM) | (VG/mL) | (%) | (pg/mL) | (pg/VG) | (NTUs) | (NTUs) |
| Cell | | | | 1.53E+12 | 100 | 13180640 | 8.61E−06 | | |
| suspension | | | | | | | | | |
| 1 | 0.32 | 0.29 | 364 | 7.30E+11 | 52 | 16535 | 2.27E−08 | 3.47 | 8.43 |
| 2 | 0.57 | 0.26 | 429 | 6.89E+11 | 52 | 44133 | 6.41E−08 | 3.73 | 5.38 |
| 3 | 0.48 | 0.29 | 249 | 6.85E+11 | 49 | 16996 | 2.48E−08 | 4.84 | 11.30 |
| 4 | 0.48 | 0.29 | 364 | 6.80E+11 | 50 | 10184 | 1.50E−08 | 3.25 | 5.64 |
| 5 | 0.48 | 0.29 | 364 | 7.31E+11 | 53 | 23783 | 3.25E−08 | 4.58 | 7.68 |
| 6 | 0.38 | 0.32 | 296 | 6.64E+11 | 47 | 7810 | 1.18E−08 | 3.95 | 9.49 |
| 7 | 0.57 | 0.32 | 296 | 6.81E+11 | 49 | 19506 | 2.86E−08 | 5.04 | 8.71 |
| 8 | 0.63 | 0.29 | 364 | 6.99E+11 | 52 | 36502 | 5.22E−08 | 4.21 | 6.39 |
| 9 | 0.48 | 0.29 | 364 | 6.80E+11 | 50 | 15675 | 2.31E−08 | 4.69 | 7.69 |
| 10 | 0.48 | 0.29 | 364 | 6.53E+11 | 48 | 11371 | 1.74E−08 | 4.00 | 6.75 |
| 11 | 0.38 | 0.32 | 429 | 6.36E+11 | 45 | 18553 | 2.92E−08 | 3.57 | 6.85 |
| 12 | 0.38 | 0.26 | 429 | 6.86E+11 | 49 | 22527 | 3.28E−08 | 3.39 | 6.33 |
| 13 | 0.57 | 0.26 | 296 | 7.38E+11 | 53 | 39537 | 5.36E−08 | 3.77 | 6.50 |
| 14 | 0.48 | 0.29 | 364 | 6.19E+11 | 44 | 26014 | 4.20E−08 | 4.21 | 7.67 |
| 15 | 0.48 | 0.34 | 364 | 6.36E+11 | 44 | 10383 | 1.63E−08 | 3.93 | 7.50 |
| 16 | 0.57 | 0.32 | 429 | 6.63E+11 | 49 | 17545 | 2.65E−08 | 4.38 | 7.01 |
| 17 | 0.48 | 0.24 | 364 | 7.44E+11 | 54 | 37583 | 5.05E−08 | 5.80 | 8.95 |
| 18 | 0.48 | 0.29 | 472 | 6.67E+11 | 50 | 18937 | 2.84E−08 | 4.01 | 6.20 |
| 19 | 0.48 | 0.29 | 364 | 6.70E+11 | 49 | 14027 | 2.09E−08 | 3.91 | 6.92 |
| 20 | 0.38 | 0.26 | 296 | 6.86E+11 | 49 | 22369 | 3.26E−08 | 3.37 | 7.53 |

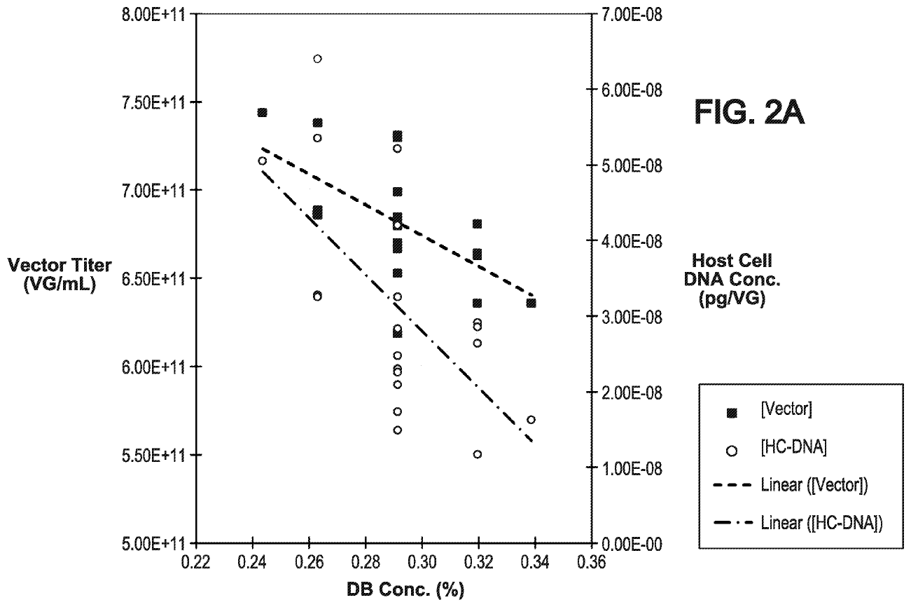

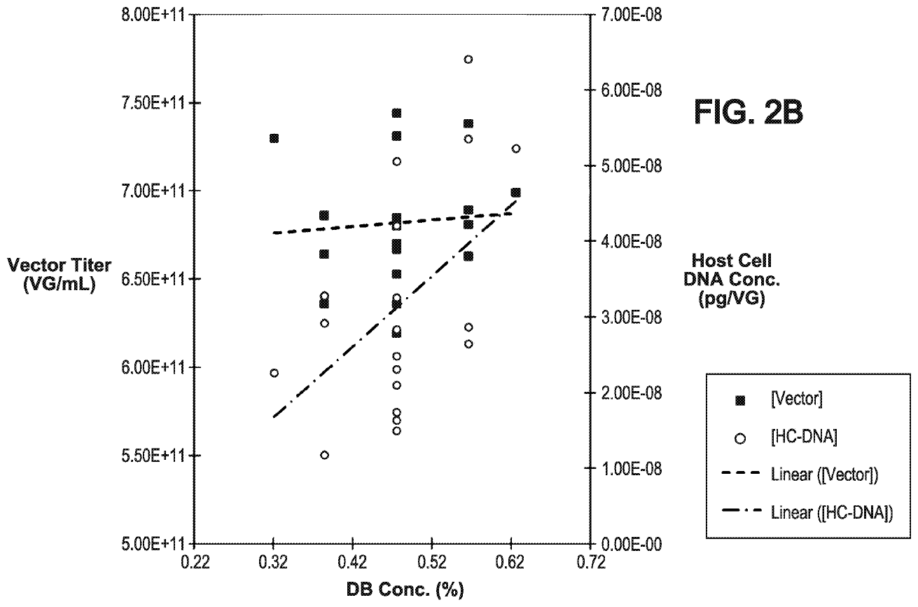

Based on the results in Table 4, FIG. 2A illustrates the relationship between the concentration of DB used to precipitate HC-DNA and the quantity of both vector and HC-DNA in samples of clarified lysates of HEK293 cells. Within the range tested, increasing DB concentration reduced both the amount of vector and HC-DNA in clarified lysates, although the rate at which HC-DNA was reduced was greater as shown by the linear trend lines fitted to the individual data points. Similarly, FIG. 2B illustrates the relationship between the concentration of Triton X-100 and the quantity of vector and HC-DNA in the same samples. Within the range tested, increasing Triton X-100 concentration increases the amount of HC-DNA in clarified lysates while not significantly affecting vector titer.

### Example 2

### Sodium Chloride Inhibits Precipitation of Residual Host Cell DNA by Domiphen Bromide

Although precipitation of host cell DNA with DB is effective to remove a substantial portion of the DNA released from lysed cells, clarified lysate contains sufficient residual HC-DNA and DB which continue to react leading to increasing turbidity with time. If not controlled, this effect can result in fouling of chromatography columns which are commonly employed in downstream processes to purify AAV vectors. Additional experiments were conducted to determine what concentration of sodium chloride would be most effective to inhibit, or quench, unwanted precipitation of residual host cell DNA by DB in clarified cell lysate.

Similar as described in Example 1, HEK293 cells were grown and transfected to produce AAV vector in two bioreactors at 3.6 L volume. When the cells had reached a viable cell densities of about 1.8×10⁷ cells/mL and 2.1×10⁷ cells/mL, respectively, the cultures were combined, Triton X-100 added (0.5% final concentration) to lyse the cells, DB added (0.2% final concentration) to precipitate HC-DNA, followed by filtration through a 1 μm depth filter (J700 (Pall)) and a 0.4 μm depth filter (Bio20 (Pall)). Samples of filtrate were then taken, including one of 2000 mL, and four of 300 mL. A solution of concentrated NaCl was then added to the filtered samples to different final concentrations ranging from 250 mM to 500 mM, followed by mixing for 30 mins at room temperature. After adding salt and mixing, the samples were filtered through 0.2 μm and 0.1 μm membrane filters (EAV and EDT, respectively (Pall)). A sample to which no NaCl was added was processed similarly and served as a control. On the same day (day 0), the samples were analyzed to quantify AAV vector titer, and amounts of HC-DNA and HCP. Vector titer was determined using qPCR and expressed as VG/mL; HCP level was determined by ELISA and expressed as ng/mL, as well as normalized to vector titer and expressed as ng/VG; and HC-DNA level was determined by qPCR and expressed as pg/mL, as well as normalized to vector titer and expressed as pg/VG. Unlike in Example 1, the samples were not further purified by chromatography. Results are reported in Table 5. As in other experiments, DB at a final concentration of 0.2% was effective to precipitate HC-DNA. In the experiment with the 2000 mL sample, the amount of HC-DNA in the clarified lysate after membrane filtration was more than 100 times less than in the untreated lysate.

TABLE 5

| | | Host Cell | Host Cell | | |
|---|---|---|---|---|---|
| | Vector Titer | Protein | Protein | Host Cell DNA | Host Cell DNA |
| Sample Condition | (VG/mL) | (ng/ml) | (ng/VG) | (pg/mL) | (pg/VG) |
| 2000 mL, 0.25M NaCl, | 2.42E+12 | 490133.51 | 2.02535E−07 | 8367647.35 | 3.45771E−06 |
| post Triton | | | | | |
| 2000 mL, 0.25M NaCl, | 6.15E+12 | 413206.03 | 6.7188E−08 | 1082954.24 | 1.7609E−07 |
| post DB | | | | | |
| 2000 mL, 0.25M NaCl, | 1.56E+12 | 413154.61 | 2.64843E−07 | 79147.47 | 5.07356E−08 |
| post DB Flocculation | | | | | |
| 2000 mL, 0.25M NaCl, | 4.65E+11 | 162110.99 | 3.48626E−07 | 3497.93 | 7.52243E−09 |
| post J700 | | | | | |
| 2000 mL, 0.25M NaCl, | 1.11E+12 | 375185.44 | 3.38005E−07 | 24933.91 | 2.2463E−08 |
| post Bio20 | | | | | |
| 2000 mL, 0.25M NaCl, | 1.01E+12 | 343906.1 | 3.40501E−07 | 30285.54 | 2.99857E−08 |
| post EAV | | | | | |
| 2000 mL, 0.25M NaCl, | 9.46E+11 | 352858.11 | 3.73E−07 | 30731.09 | 3.24853E−08 |
| post EDT | | | | | |
| 300 mL, 0M NaCl | 1.17E+12 | 365081.69 | 3.12036E−07 | 18364.73 | 1.56964E−08 |
| 300 mL, 0.25M NaCl | 1.2E+12 | 360253.24 | 3.00211E−07 | 20265.99 | 1.68883E−08 |
| 300 mL, 0.4M NaCl | 1.35E+12 | 351993.51 | 2.60736E−07 | 26215.4 | 1.94188E−08 |
| 300 mL, 0.5M NaCl | 1.02E+12 | 337888.83 | 3.31264E−07 | 26073.21 | 2.5562E−08 |

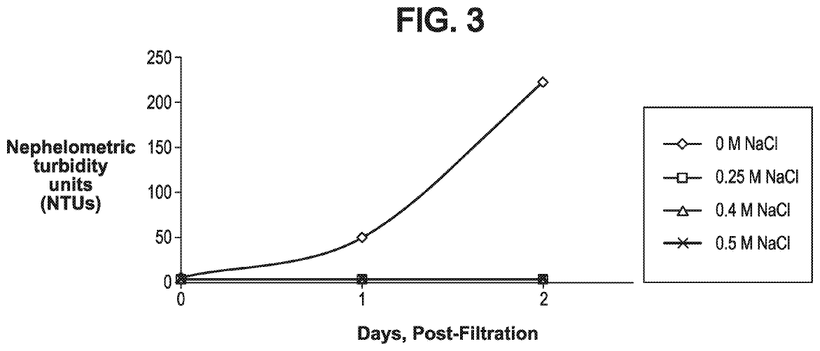

Turbidity was also measured on days 0, 1 and 2 using a turbidimeter, and results expressed in nephelometric turbidity units (NTUs). Results are reported in Table 6 and illustrated in FIG. 3. As shown in FIG. 3 when no salt (0 M NaCl) was added to lysate, turbidity increased nearly 40-fold over the course of the 2 day experiment, which indicated that precipitation of residual HC-DNA by DB was an ongoing process. By contrast, addition of NaCl to a final concentration ranging from 0.25 M to 0.50 M resulted in turbidity levels that were essentially unchanged over the same time, indicating that addition of salt at these concentrations was effective to inhibit the ongoing precipitation of residual HC-DNA by DB. As shown in Table 5, AAV vector titers were comparable across all salt concentrations tested (including the no salt control), as was the amount of host cell protein.

TABLE 6

| | NaCl final | | | |
|---|---|---|---|---|
| Sample Condition | conc. mM | NTUs Day 0 | NTUs Day 1 | NTUs Day 2 |
| 300 mL | 0 | 5.6 | 49 | 222 |
| 300 mL | 250 | 3.1 | 4 | 4.19 |
| 300 mL | 400 | 3 | 3.2 | 4 |
| 300 mL | 500 | 3 | 3.4 | 4 |

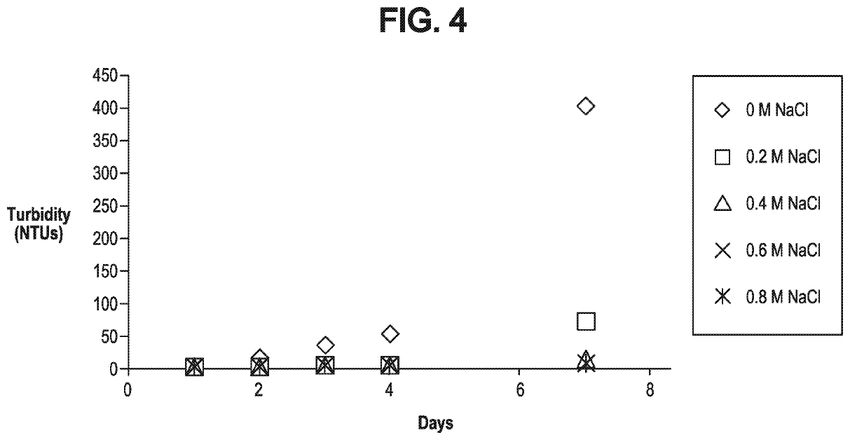

In another series of experiments, HEK293 cells were grown, transfected for AAV vector production and harvested similarly as in other experiments described above. Cells were lysed with 0.5% Triton X-100 (30 mins at room temperature), host cell DNA precipitated with 0.3% DB (30 mins at room temperature), and then filtered through J700, Bio20, EAV, and EDT filters as described herein. A concentrated salt solution was then added to the samples to achieve a final NaCl concentration ranging from 200 mM to 800 mM, with a no salt control. A portion of the treated samples were then held for 7 days and turbidity measured on days 1, 2, 3, 4, and 7, and the other portion purified by immunoaffinity column chromatography to purify AAV vector. Results of the turbidity measurements are reported in Table 7 which, as is further illustrated in FIG. 4, demonstrate that NaCl concentrations greater than 200 mM were effective to prevent significant increases in turbidity even after 7 days.

TABLE 7

| NaCl final | NTUs | NTUs | NTUs | NTUs | NTUs |
|---|---|---|---|---|---|
| conc. mM | Day 1 | Day 2 | Day 3 | Day 4 | Day 5 |
| 0 | 2.6 | 16.2 | 35.1 | 52.2 | 404 |
| 200 | 1.93 | 2.04 | 3.25 | 4.58 | 73.4 |
| 400 | 1.88 | 2.13 | 3.02 | 3.85 | 15.1 |
| 600 | 2.16 | 2.24 | 2.98 | 3.78 | 7.76 |
| 800 | 2.13 | 2.22 | 3.01 | 3.71 | 6.11 |

After isolating AAV vector from the different salt treated samples by immunoaffinity column chromatography, the purified preparations were analyzed to determine vector titer by qPCR, the proportion of full capsids by measuring absorbance at 260 nm and 280 nm and calculating the A260/A280 ratio, and to quantify the amount of HC-DNA and HCP. Results are reported in Table 8, which demonstrate no significant differences in vector titer and purity or in the amounts of HC-DNA or HCP in the purified vector preparations.

TABLE 8

| NaCl final conc. | Titer | | HC-DNA (ng/ | HCP (μg/ |
|---|---|---|---|---|
| mM | (vg/ml) | A260/A280 | 1E14 VG) | 1E14 VG) |
| 0 | 2.14E+12 | 1.11 | 414 | 24,446 |
| 200 | 1.80E+12 | 1.28 | 480 | 25,380 |
| 400 | 1.60E+12 | 1.17 | 415 | 31,315 |
| 600 | 1.60E+12 | 1.18 | 293 | 30,236 |
| 800 | 1.43E+12 | 1.19 | 369 | 31,675 |

### Example 3

Effect of Anion and Cation Replacement on the Inhibition of Residual Host Cell DNA Precipitation by Domiphen Bromide

While not wishing to be bound by theory, a possible explanation of the effect of sodium chloride on HC-DNA precipitation by DB is that the chloride anion electrostatically interacts with the positively charged domiphen moiety, shielding it and reducing its interaction with negatively charged DNA. To further investigate this potential mechanism, a series of experiments were conducted that tested the effect of different salts on precipitation of HC-DNA and AAV vector production. Test conditions included a wider range of NaCl concentration than tested in Example 2, use of salts in which sodium was paired with different inorganic and organic anions, use of salts in which chloride was paired with different inorganic cations, and a salt (MgSO₄) in which neither sodium nor chloride was present.

As in previous examples, HEK293 cells were grown and transfected for AAV vector production. When cells reached a viable cell density of about 1.4×10⁷ cells/mL, cells were lysed with Triton X-100 at a final concentration of 0.5% for 30 minutes. After lysis, DB was added to final concentration of 0.3% with mixing for 30 minutes to precipitate HC-DNA. Flocculated HC-DNA was allowed to settle, after which supernatant was pumped out of the bioreactor and clarified by filtration through 19 μm and 0.4 μm depth filters. To 300 mL samples of the clarified lysate, stock solutions containing 0.5% Triton X-100 and salts were added as described in Table 9, mixed, and filtered through 0.22 μm and 0.1 μm filters to produce final clarified lysates which were then dispensed into storage bottles and held at room temperature for 4 days. On day 0, samples were analyzed to measure pH, conductivity, AAV vector titer, and concentrations of HC-DNA and HCP, whereas turbidity was measured with a Hach 2100 Q turbidimeter on days 0-4 before and after filtration through 0.2 μm and 0.1 μm filters.

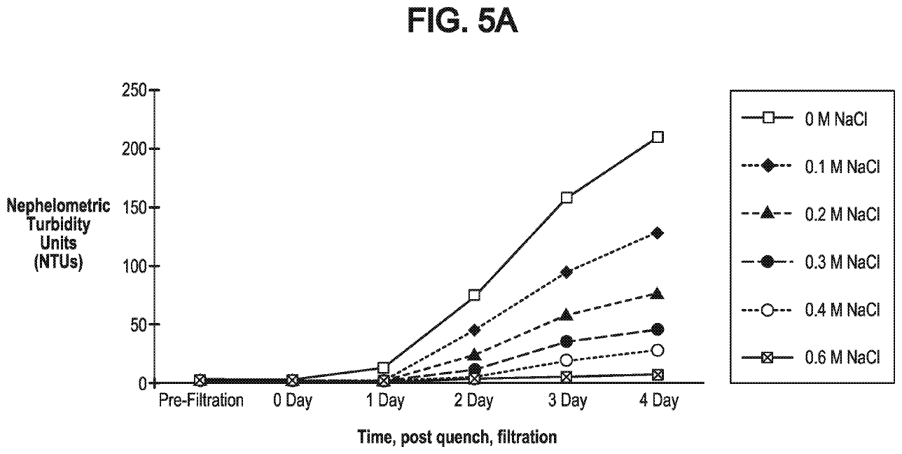

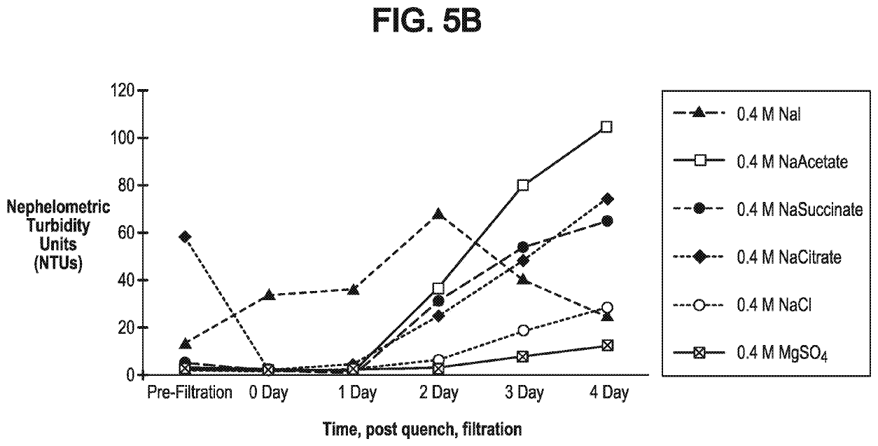

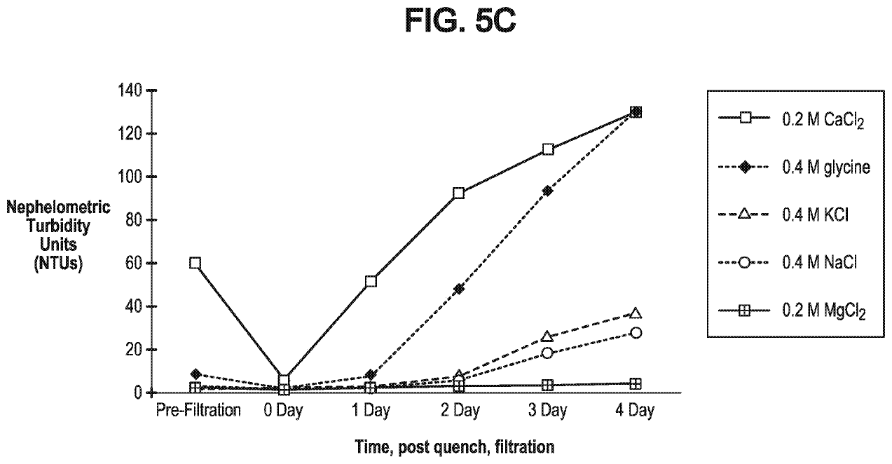

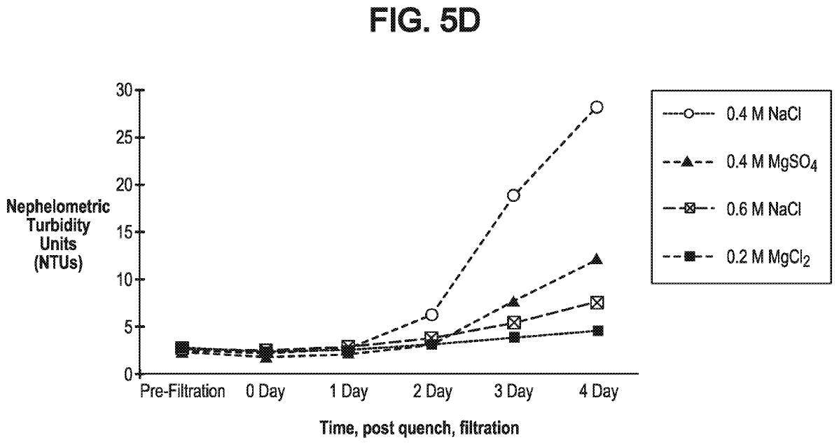

Table 10 reports the results of turbidity measurements reported in nephelometric turbidity units (NTUs) (average of 3 measurements) over time in the presence of the different salts, which are also illustrated in the graphs in FIGS. 5A-5D. Table 9 reports the results of measurements on day 0 of lysate conductivity, whereas Table 11 reports the results of measurements on the same day of AAV vector titer, and normalized concentrations of HCP and HC-DNA in the lysate samples.

TABLE 9

| | | | Inhibitor | | | | | |
|---|---|---|---|---|---|---|---|---|
| | | | Stock | mL Inhibitor | | | | Conductivity |
| | | | Solution in | Added to 300 | Final | Final | pH, | (mS/cm), |
| | | Inhibitor | 0.5% Triton | mL Clarified | Inhibitor | Anion | Post | Post |
| Group | Rationale | Condition | X-100 | Lysate | Molarity | Molarity | Filtration | Filtration |
| NaCl | Negative Control | 0M NaCl | 0M NaCl | 30 | 0.00 | 0.00 | 7.37 | 10.7 |
| Gradient | 0.1M NaCl | 0.1M NaCl | 1M NaCl | 30 | 0.09 | 0.09 | 7.43 | 17.8 |
| | 0.2M NaCl | 0.2M NaCl | 2M NaCl | 30 | 0.18 | 0.18 | 7.33 | 25.6 |
| | 0.3M NaCl | 0.3M NaCl | 3M NaCl | 30 | 0.27 | 0.27 | 7.36 | 32.2 |
| | 0.4M NaCl | 0.4M NaCl | 4M NaCl | 30 | 0.36 | 0.36 | 7.33 | 38.8 |
| | 0.6M NaCl | 0.6M NaCl | 4M NaCl | 50 | 0.57 | 0.57 | 7.29 | 55.3 |
| Anion | Halogen Replacement | 0.4M NaI | 4M NaI | 30 | 0.36 | 0.36 | 7.34 | 41.9 |
| Replacement | Monovalent Organic | 0.4M NaAcetate | 4M NaAcetate* | 30 + 10 | 0.38 | 0.38 | 7.37 | 27.7 |
| | Anion | | | AcOH* | | | | |
| | Divalent Organic | 0.4M NaSuccinate | 1M NaSuccinate | 170 | 0.36 | 0.36 | 7.65 | 41.5 |
| | Anion | | | | | | | |
| | Trivalent Organic Anion | 0.4M NaCitrate | 1.5M NaCitrate** | 95 + 10 C.A.** | 0.39 | 0.39 | 6.97 | 43.3 |
| Anion | Divalent, Chaotropic | 0.4M MgSO₄ | 2.5M MgSO₄ | 50 | 0.36 | 0.36 | 7.20 | 28.9 |
| Replacement + | Cation + Divalent Anion | | | | | | | |
| Cation | | | | | | | | |
| Replacement | | | | | | | | |
| Cation | Group 1 replacement | 0.4M KCl | 4M KCl | 30 | 0.36 | 0.36 | 7.41 | 47.0 |
| Replacement | Group 2 replacement | 0.2M MgCl₂ | 2M MgCl₂ | 30 | 0.18 | 0.36 | 7.24 | 33.9 |
| | Group 2 replacement | 0.2M CaCl₂ | 2M CaCl₂ | 30 | 0.18 | 0.36 | 7.06 | 35.7 |
| Amino Acid | Glycine | 0.4M glycine | 2M glycine | 65 | 0.36 | N/A | 7.34 | 8.9 |

*Addition of 30 mL of 4M NaAcetate in 0.5% Triton X-100 resulted in a solution with pH 9.7. The solution was neutralized to pH 7.37 by addition of 10 mL of 1M acetic acid (AcOH) in 0.5% Triton X-100.
**Addition of 95 mL of 1.5M NaCitrate in 0.5% Triton X-100 resulted in a solution with pH 8.4. The solution was neutralized to pH 6.97 by addition of 10 mL of 1.5M citric acid (C.A.) in 0.5% Triton X-100.

TABLE 10

| | 0 Day, Pre- | 0 Day, Post | 1 Day, Post | 2 Day, Post | 3 Day, Post | 4 Day, Post |
|---|---|---|---|---|---|---|
| Inhibitor | Filtration | Filtration | Filtration | Filtration | Filtration | Filtration |
| Concentration | NTUs | NTUs | NTUs | NTUs | NTUs | NTUs |
| 0M NaCl | 2.7 | 2.8 | 12.8 | 74.9 | 158.3 | 210.0 |
| 0.1M NaCl | 2.2 | 1.8 | 2.6 | 45.5 | 95.1 | 128.3 |
| 0.2M NaCl | 2.2 | 1.9 | 2.5 | 24.9 | 58.3 | 76.9 |
| 0.3M NaCl | 2.2 | 2.0 | 2.5 | 12.1 | 35.4 | 46.7 |
| 0.4M NaCl | 2.4 | 2.2 | 2.7 | 6.3 | 18.9 | 28.3 |
| 0.6M NaCl | 2.7 | 2.5 | 2.9 | 3.8 | 5.5 | 7.6 |
| 0.4M NaI | 13.3 | 33.9 | 36.1 | 68.4 | 40.4 | 24.7 |
| 0.4M NaAcetate | 2.8 | 2.1 | 2.8 | 36.4 | 79.7 | 104.7 |
| 0.4M NaSuccinate | 5.2 | 1.6 | 2.2 | 31.3 | 54.1 | 65.0 |
| 0.4M NaCitrate | 58.3 | 1.4 | 4.9 | 24.9 | 48.4 | 74.3 |
| 0.4M MgSO₄ | 2.3 | 1.8 | 2.1 | 2.9 | 7.7 | 12.2 |
| 0.4M KCl | 3.3 | 2.5 | 3.0 | 8.2 | 26.3 | 37.0 |
| 0.2M MgCl₂ | 2.8 | 2.4 | 2.7 | 3.2 | 3.9 | 4.6 |
| 0.2M CaCl₂ | 60.0 | 6.9 | 51.8 | 92.5 | 113.0 | 130.0 |
| 0.4M glycine | 9.2 | 1.9 | 8.4 | 48.3 | 93.8 | 130.0 |

As shown in FIG. 5A with additional detail in Table 10, clarified lysate demonstrated increasing turbidity after one day, which was inhibited in a concentration dependent manner as higher concentrations of NaCl were added. In these experiments, 0.6 M NaCl appeared to nearly completely inhibit further DB precipitation of residual HC-DNA.

Interestingly, as shown in FIG. 5B, salts comprising sodium and anions other than chloride did not perform better than NaCl when tested at the same concentration (0.4 M) at preventing gradual turbidity increase. Furthermore, two of the salts tested, NaI and sodium citrate, appeared to affect turbidity in surprising ways over time. The salt NaI appeared to dramatically accelerate turbidity initially, but inhibit it later, whereas sodium citrate appeared to accelerate turbidity initially followed by inhibition that gradually diminished at later time points. Chloride has a higher charge density than the anions that were less effective at inhibiting turbidity, suggesting that charge density may be an important variable affecting interaction with positively charged domiphen. Notably, also as shown in FIG. 5B, MgSO₄ was more effective for inhibiting turbidity than NaCl at the same concentration. Since chloride has a higher charge density than sulfate, the observed difference could be attributable to strong electrostatic interaction of Mg²⁺ ions with negatively charged DNA, which might interfere with the interaction of DNA with DB.

Two salts tested comprising chloride and cations other than sodium performed no better than NaCl for inhibiting turbidity, as shown in FIG. 5C. After initially appearing to inhibit turbidity, 0.2 M CaCl₂ caused turbidity to rapidly increase at later times, whereas KCl was effective, but slightly less so compared to NaCl at the same concentration. In contrast, when chloride was paired with Mg²⁺ counterions, inhibition of turbidity was more effective than NaCl even at one-half the concentration (0.2 M). This effect could be attributable to the high charge density of chloride (possibly contributing to more effective interaction with domiphen) combined with strong Mg²⁺ interaction with DNA. The amino acid glycine, which can behave as a zwitterion, was also tested but failed to inhibit turbidity to any appreciable extent. FIG. 5D provides additional detail regarding the comparative effectiveness of the most potent inhibitors of turbidity formation.

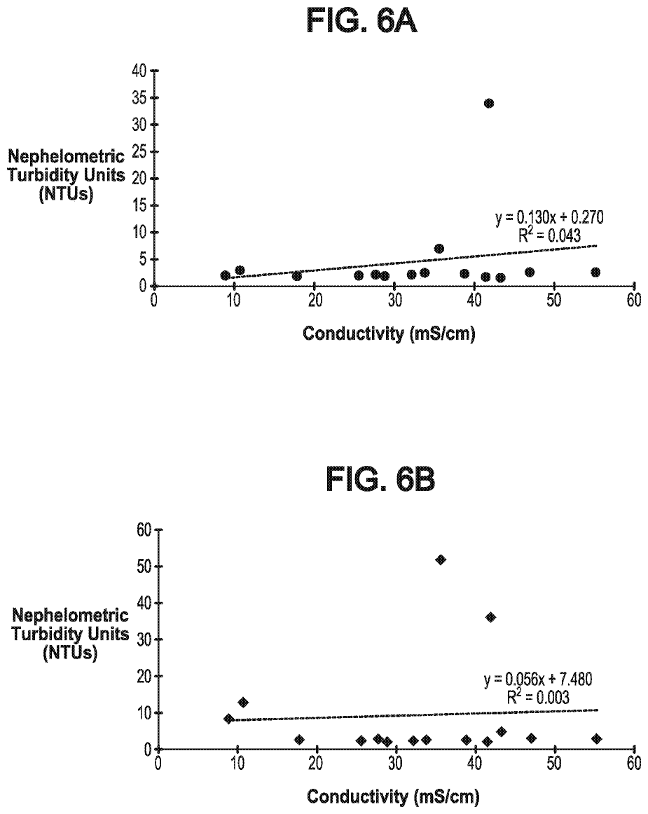

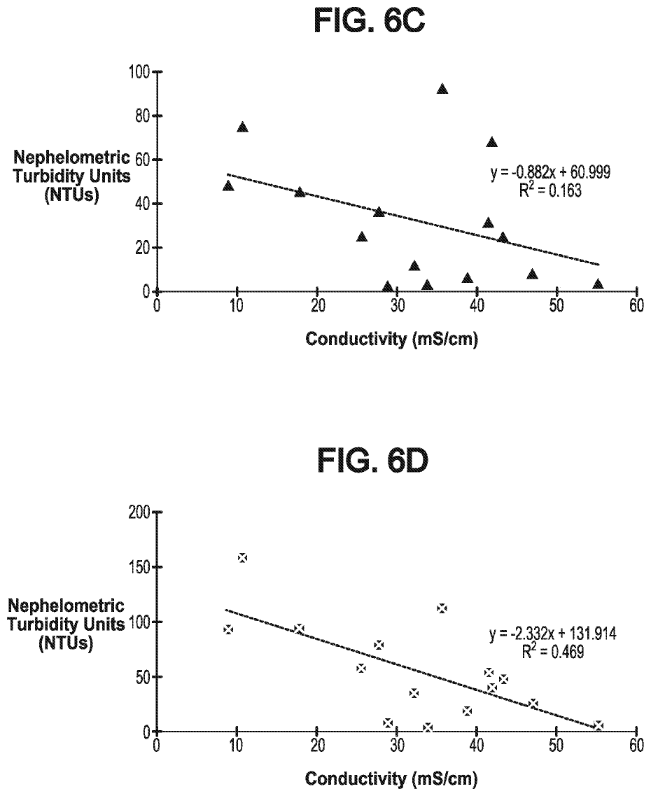

Since different amounts of the same salt, or similar amounts of different salts, can contribute to ionic strength to variable degrees, it was possible that inhibition of turbidity formation might be due to ionic strength instead of the chemical nature of the various ions and their interaction with DB and/or DNA. As shown in FIGS. 6A-6D, regression plots of turbidity over the test period (days 0-3, as reported in Table 10) as a function of conductivity (at day 0 as reported in Table 9), however, indicates that ionic strength is poorly predictive at early time points, and at most weakly predictive at later time points. Only at day 3 did regression analysis suggest that higher ionic strength might be positively correlated with a reduction in turbidity formation. Collectively, this data suggests that the chemical properties of the ions in the salts tested are a more significant contributing factor to turbidity inhibition than ionic strength.

As summarized in Table 11, addition of most precipitation inhibitors did not significantly affect AAV vector titers as reflected in the inhibition % VG yield values, which exceeded 100%. In contrast, addition of 0.4 M MgSO₄, 0.2 M MgCl₂, and 0.2 M CaCl₂) caused a reduction in post-filtration inhibition % VG yields. The data in Table 11 also demonstrates that DB is highly effective a removing HC-DNA from lysed cells (from 770.9 pg/1×10⁹ VG before adding DB to 50.6 pg/1×10⁹ VG after). Addition of the inhibitors followed by 0.2 μm and 0.1 μm filtrations did not significantly alter HC-DNA levels.

TABLE 11

| | Inhibition % VG Yield* | | Total % VG Yield^{†} | | HC-DNA (pg)/1 × 10⁹ VG | | HCP (ng)/1 × 10⁹ VG | |
|---|---|---|---|---|---|---|---|---|
| | (qPCR) | | (qPCR) | | (qPCR) | | (ELISA) | |
| Step or Inhibitor | Pre- | Post | Pre- | Post | Pre- | Post | Pre- | Post |
| Condition | Filtration | Filtration | Filtration | Filtration | Filtration | Filtration | Filtration | Filtration |
| Post Triton Lysis | N/A | N/A | 100 | N/A | 770.9 | N/A | 239 | N/A |
| Post DB Floc. | N/A | N/A | 198 | N/A | 50.6 | N/A | 119 | N/A |
| Post Bio 20 Filt. | N/A | 100 | N/A | 17 | N/A | 33.1 | N/A | 1048 |
| 0M NaCl | 108 | 104 | 18 | 18 | ≤32.9 | ≤34.1 | 1007 | 980 |
| 0.1M NaCl | 120 | 108 | 20 | 18 | ≤29.6 | ≤32.8 | 761 | 985 |
| 0.2M NaCl | 106 | 120 | 18 | 20 | ≤3.3 | ≤29.6 | 896 | 764 |
| 0.3M NaCl | 111 | 105 | 19 | 18 | ≤32.1 | ≤33.8 | 771 | 1077 |
| 0.4M NaCl | 111 | 101 | 19 | 17 | F.A. | F.A. | 931 | 999 |
| 0.6M NaCl | 103 | 107 | 17 | 18 | 8.8 | 10.5 | 1087 | 929 |
| 0.4M NaI | 102 | 128 | 17 | 22 | 44.2 | ≤27.8 | 952 | 790 |
| 0.4M NaAcetate | 128 | 110 | 22 | 19 | 32.5 | ≤33.3 | 759 | 839 |
| 0.4M | 108 | 162 | 18 | 27 | ≤4.7 | 11.3 | 929 | 588 |
| NaSuccinate | | | | | | | | |
| 0.4M NaCitrate | 142 | 125 | 24 | 21 | 34.1 | ≤34.7 | 676 | 660 |
| 0.4M MgSO₄ | 59 | 62 | 10 | 10 | 68.1 | ≤61.0 | 1124 | 1125 |
| 0.4M KCl | 103 | 135 | 17 | 23 | F.A. | 27.9 | 857 | 655 |
| 0.2M MgCl₂ | 82 | 64 | 14 | 11 | ≤43.5 | ≤55.9 | 1010 | 1164 |
| 0.2M CaCl₂ | 49 | 56 | 8 | 10 | 18.2 | F.A. | 1153 | 928 |
| 0.4M glycine | 129 | 87 | 22 | 15 | 7.5 | ≤45.2 | 596 | 1118 |

*Inhibition % VG Yield = (Total VG in inhibited solution)/(Total VG in Post Bio 20 filtrate)
^{†}Total % VG Yield = (Total VG in inhibited solution)/(Total VG in Post Triton Lysis solution)

### Example 4

### Inhibition of Residual Host Cell DNA Precipitation by Domiphen Bromide at Large Scale

The studies of DB precipitation of HC-DNA and its inhibition by addition of certain salts described above were conducted at relatively small scale, and relied on batch mixing techniques to combine clarified cell lysate with the salt solutions. Batch mixing, however, would be inefficient if applied to vector manufacturing at clinical or commercial scales, and experiments were conducted to test the effectiveness of a semi-continuous system for mixing DB treated cell lysate with concentrated salt solution and comparing it to a batch system of the same scale. The batch system is illustrated in FIG. 7B and the continuous system in FIG. 7C. An alternative system for continuous mixing is illustrated in FIG. 7A.

Experiments employing either the batch or continuous process system were performed. HEK293 cells were grown in suspension culture into a 250 L single use bioreactor (SUB) and transfected with plasmids for AAV vector production essentially as described in the examples above. About 68 hours after transfection, at which time the viable cell density had reached about 2.0×10⁷ cells/mL, 10% Triton X-100 was added to the SUB to a final concentration of 0.5% followed by mixing for 30 minutes. To precipitate HC-DNA, 10% domiphen bromide was added to the SUB to a final concentration of 0.3% followed by mixing for 30 minutes. The SUB impeller was turned off and flocculated HC-DNA allowed settled to the bottom of the SUB.

To quench precipitation of residual HC-DNA by DB in the batch system, partially clarified lysate overlying the layer of DB-precipitated HC-DNA was pumped out of the SUB and through 1.0 μm and a 0.4 μm depth filters (T3500 and Bio 20 Stax, respectively) into a single use mixer (SUM). A concentrated salt solution containing 4 M NaCl and 0.5% Triton X-100 was then pumped into the SUM followed by mixing with the filtrate to allow the salt to inhibit further HC-DNA precipitation. The quenched mixture was then pumped out of the SUM and passed through additional filters. In the continuous mixing system, the partially clarified lysate was pumped out of the SUB and filtered as above, and then stored temporarily in the SUM, which in this design serves as break tank. After a sufficient volume of filtered supernatant was added to the SUM, supernatant and a solution containing 4 M NaCl and 0.5% Triton X-100 stored in a separate tank were pumped out of their respective containers by peristaltic pumps through separate tubes meeting at a Y-connector. In these experiments, it was desired to achieve a final concentration of 0.4 M NaCl in the mixture. Accordingly, the relative pump rates for filtrate and concentrated NaCl solution was about 10:1. Downstream of the Y-connector was a single tube containing a static in-line mixing element to ensure thorough mixing of the filtrate and salt solution, after which the mixture was passed through additional filters.

In practice, the supernatant and salt solution mixture would be subjected to further downstream purification steps (e.g., chromatography), but in these experiments was stored at room temperature to monitor turbidity changes over time. AAV vector titer was determined on day 0 and turbidity measured on days 0-4 using a Hach 2100 Q turbidimeter. Aspects of the experimental systems are summarized in Table 12 and results summarized in Table 13. A control sample to which no precipitation inhibitor was added was included, as was one test sample prepared using the batch system, and three test samples prepared using the continuous system configured with different pump flow rates, tubing diameters and static mixers. Consistent with results discussed above, the control to which no NaCl was added exhibited increasing turbidity with time. By contrast, when NaCl was added to clarified lysate using either the batch or continuous systems, no significant increase in turbidity levels were observed by the last day of the experiment. AAV titer levels were also consistent across the different experiments. This data suggests that a continuous mixing system would be as effective as a batch system to mix clarified lysate with salt solutions to inhibit precipitation of residual HC-DNA by DB before storage, and/or use in downstream processes to further purify AAV vector.

TABLE 12

| | | NaCl | | Total | | |
|---|---|---|---|---|---|---|
| | Filtrate | Solution | | Volume | Post-Y- | Static In-Line |
| | Pump | Pump | Total | NaCl | Connector | Mixing |
| Sample | Rate | Rate | Volume | Solution | Tubing ID | Element |
| Condition | (mL/min) | (mL/min) | Filtrate | (mL) | (in.) | Properties |
| Batch mixing | N/A | N/A | N/A | N/A | N/A | N/A |
| control | | | | | | |
| (no NaCl | | | | | | |
| added) | | | | | | |
| Batch mixing | N/A | N/A | N/A | N/A | N/A | N/A |
| w/NaCl | | | | | | |
| added | | | | | | |
| Continuous | 100 | 10 | 2000 | 200 | 0.25 | 16 mixing |
| mixing | | | | | | elements |
| w/NaCl | | | | | | 6.35 mm OD |
| added | | | | | | 101.6 mm |
| (Experiment | | | | | | total length |
| 1) | | | | | | 1.000 L/D |
| | | | | | | ratio |
| Continuous | 400 | 40 | 5000 | 500 | 0.25 | 16 mixing |
| mixing | | | | | | elements |
| w/NaCl | | | | | | 6.35 mm OD |
| added | | | | | | 101.6 mm |
| (Experiment | | | | | | total length |
| 2) | | | | | | 1.000 L/D |
| | | | | | | ratio |
| Continuous | 1000 | 100 | 10000 | 1000 | 0.375 | 24 mixing |
| mixing | | | | | | elements |
| w/NaCl | | | | | | 9.47 mm OD |
| added | | | | | | 198.1 mm |
| (Experiment | | | | | | total length |
| 3) | | | | | | 0.872 L/D |
| | | | | | | ratio |

TABLE 13

| | AAV Vector | | | | | |
|---|---|---|---|---|---|---|
| | Titer | Day 0 | Day 1 | Day 2 | Day 3 | Day 4 |
| Sample Condition | (VG/mL) | NTUs | NTUs | NTUs | NTUs | NTUs |
| Batch mixing control | 1.49 × 10¹¹ | 2.1 | 9.07 | 30.3 | 73.1 | 102 |
| (no NaCl added) | | | | | | |
| Batch mixing | 1.14 × 10¹¹ | 2.73 | 2.79 | 2.84 | 2.84 | 3.18 |
| w/NaCl added | | | | | | |
| Continuous mixing | 1.45 × 10¹¹ | 2.94 | 2.81 | 2.73 | 2.87 | 3.23 |
| w/NaCl added | | | | | | |
| (Experiment 1) | | | | | | |
| Continuous mixing | 1.43 × 10¹¹ | 2.73 | 2.74 | 2.69 | 2.84 | 3.25 |
| w/NaCl added | | | | | | |
| (Experiment 2) | | | | | | |
| Continuous mixing | 1.30 × 10¹¹ | 2.70 | 2.69 | 2.63 | 2.74 | 3.23 |
| w/NaCl added | | | | | | |
| (Experiment 3) | | | | | | |

### Example 5

### Effect of Inhibiting Domiphen Bromide DNA Precipitation on Immunoaffinity Column Chromatography Efficiency

Using similar methods and reagents as described in the previous examples, separate preparations of an AAV vector with an AAV9 capsid were produced at 250 L scale by transfecting HEK293 cells in suspension culture. Vector was harvested by lysing cells with Triton X-100 and then precipitating host cell DNA (HC-DNA) with domiphen bromide at a final concentration of Flocculated HC-DNA was allowed to settle after which partially clarified cell lysate containing vector was removed and depth filtered to produce clarified lysate. Salt was not added to inhibit precipitation of residual host cell DNA. Vector was then purified by immunoaffinity chromatography using a column packed with POROS™ CaptureSelect™ AAV9 Affinity Resin (Thermo Fisher Scientific). Clarified lysate from multiple vector preparations was run through the same column to assess how many purification cycles were possible before column performance degraded to an undesirably low level. The amount of vector before and after purification from each preparation was determined by qPCR and the yield calculated by dividing the amount of vector in the combined elution pool by the amount of vector in the clarified lysate before chromatography.

Results are shown in Table 14 which demonstrate a rapid degradation of column performance to 30% vector yield after only four purification cycles. The total amount of purified vector from each purification cycle varies because different vector preparations were being purified, each containing different amounts of vector. Before the fifth purification cycle, the immunoaffinity resin was removed from the column, washed, and repacked to test if this could improve resin performance, but only a small improvement in yield was observed.

TABLE 14

| Column Use | Total AAV Vector After Immunoaffinity | Vector |
|---|---|---|
| Cycle No. | Chromatography (VG) | Yield |
| 1 | 2.94 × 10¹⁶ | 103% |
| 2 | 5.00 × 10¹⁶ | 88% |
| 3 | 2.33 × 10¹⁶ | 49% |
| 4 | 1.96 × 10¹⁶ | 30% |
| 5 | 8.16 × 10¹⁵ | 40% |

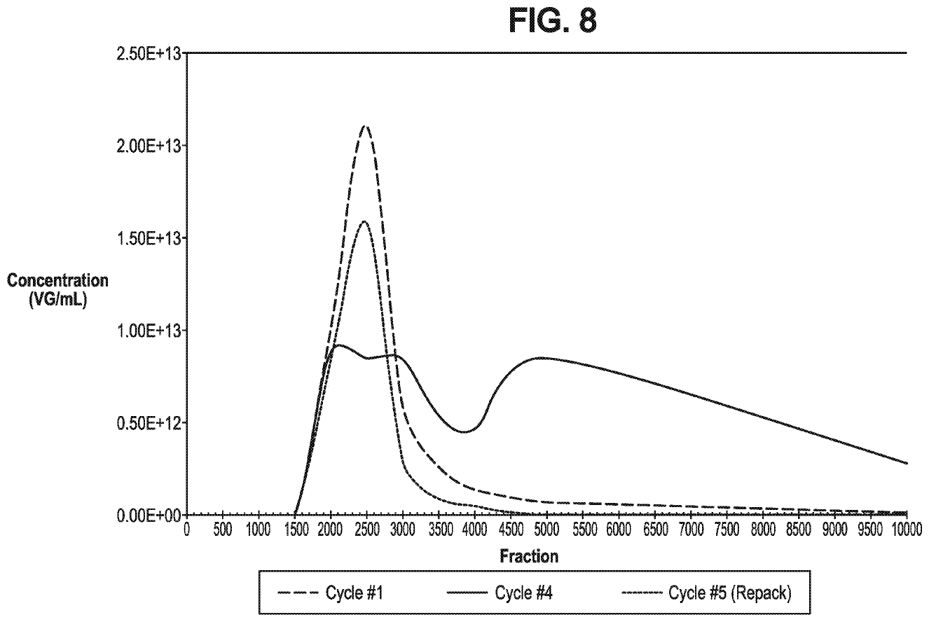

Column performance was also assessed by quantifying the amount of vector in serial elution fractions during the elution phase of immunoaffinity chromatography purification. Results from three of the purification cycles described in Table 14 are shown in the chromatogram of FIG. 8. The first cycle (Cycle #1) shows a single sharp elution profile peak indicating effective separation of the vector by the immunoaffinity resin. By the fourth cycle (Cycle #4), however, the elution peak was broad and bimodal, indicating degradation of column performance. Washing and repacking the resin was effective to restore the ability of the resin to separate vector, but with reduced yield (Cycle #5).

The effect of adding salt to clarified lysate on immunoaffinity chromatography performance was also assessed. AAV vector with AAV9 capsid was produced in HEK293 cells in suspension culture as described in other examples, after which the cells were lysed with Triton X-100 and host cell DNA precipitated with domiphen bromide. After depth filtration, NaCl was added to the clarified lysate to a final concentration of 0.4 M (not accounting for NaCl that may have already been present). A column packed with POROS™ CaptureSelect™ AAV9 Affinity Resin was then used to purify vector from the lysate through 10 cycles of sample loading, washing, vector elution, and cleaning in place of the resin. Vector yield was determined as above, vector purity was determined by non-denaturing reverse phase HPLC, and column pressure required to maintain a constant flow rate was also monitored. Results are shown in Table 15. With salt treatment, column performance was consistently high in terms of vector yield and purity, although column pressure did increase over the course of the 10 purification cycles (max delta pressure approximately doubling). As compared to the data in Table 14, a new clean in place protocol was implemented for these experiments, which may also have contributed to the improved performance.

TABLE 15

| | | | Column |
|---|---|---|---|
| Column Use | Vector | Vector | Pressure |
| Cycle No. | Yield | Purity | (MPa) |
| 1 | 68% | 91% | 0.19 |
| 2 | 76% | 90% | 0.19 |
| 3 | 108% | 89% | 0.20 |
| 4 | 103% | 89% | 0.21 |
| 5 | 92% | 89% | 0.23 |
| 6 | 96% | 89% | 0.27 |
| 7 | 92% | 89% | 0.31 |
| 8 | 84% | 89% | 0.31 |
| 9 | 77% | 89% | 0.40 |
| 10 | 79% | 89% | 0.38 |

Collectively, the results shown in Table 14, FIG. 8 and Table 15 suggest that inhibiting precipitation of residual host cell DNA by domiphen bromide in clarified host cell lysates by salt addition is effective to increase the number of AAV vector purification cycles that a chromatography column is capable of undergoing before performance falls below an undesirable level.

What is claimed is:

1. A method of removing host cell DNA from a sample of lysed host cells, comprising the steps of (i) lysing the host cells, producing a lysate, (ii) precipitating host cell DNA from the lysate, producing a flocculant and a supernatant (iii) separating the supernatant from the flocculant, and (iv) inhibiting precipitation of residual host cell DNA in the supernatant.

2. The method of claim 1, wherein the host cells are suspended in a physiologically compatible fluid, forming a cell suspension, and are lysed by adding to the cell suspension a solution comprising a detergent in a concentration sufficient to cause cell lysis.

3. The method of claim 2, further comprising mixing the cell suspension and detergent solution.

4. The method of any one of claims 1-3, wherein prior to being suspended in a physiologically compatible fluid, the host cells are grown or maintained as an adherent cell culture on a substrate, or in suspension cell culture.

5. The method of any one of claims 2-4, wherein the detergent is an ionic detergent, a non-ionic detergent, or a zwitterionic detergent.

6. The method of claim 5, wherein the non-ionic detergent is selected from the group of detergent compounds consisting of alkylphenol ethoxylate, 4-alkylphenol ethoxylate, octylphenol ethoxylate, 4-octylphenol ethoxylate, nonylphenol ethoxylate, 4-nonylphenol ethoxylate, Triton X-100, Triton X-114, NP-40, Tween 20, and Tween 80.

7. The method of claim 6, wherein the non-ionic detergent is Triton X-100.

8. The method of any one of claim 2-7, wherein prior to lysis, the viable cell density of the host cells in the cell suspension is at least about 10×10⁶ vc/mL.

9. The method of claim 8, wherein the viable cell density of the host cells ranges from about 10×10⁶ to 30×10⁶ vc/mL, or from about 15×10⁶ to 25×10⁶ vc/mL.

10. The method of any one of claims 1-9, wherein the host cells are mammalian cells or insect cells.

11. The method of claim 10, wherein the host cells are selected from the group of cells consisting of HEK293 cells, CHO cells, HeLa cells, Sf9 cells, and Sf1 cells.

12. The method of any one of claims 2-11, wherein the final concentration of detergent in the lysate is at least 0.3%.

13. The method of claim 12, wherein the final concentration of detergent in the lysate ranges from about 0.3% to 0.7%, or from about 0.4% to 0.6%.

14. The method of claim 13, wherein the final concentration of detergent in the lysate is about 0.5%.

15. The method of any one of claims 1-12, wherein host cell DNA in the lysate is precipitated by adding to the lysate a solution comprising a domiphen halide.

16. The method of claim 15, further comprising mixing the lysate and the solution comprising the domiphen halide.

17. The method of any one of claims 15-16, wherein the domiphen halide is domiphen bromide (DB).

18. The method of claim 17, wherein the final concentration of DB in the lysate is at least 0.15%.

19. The method of claim 18, wherein the final concentration of DB in the lysate ranges from about 0.15% to 0.45%, about 0.2% to 0.4%, or about 0.2% to 0.3%.

20. The method of claim 19, wherein the final concentration of DB in the lysate is about 0.3%.

21. The method of any one of claims 17-18, wherein the final concentration of DB in the lysate relative to the viable cell density prior to lysis is not less than 0.009%, 0.008%, or 0.007% per 1×10⁶ vc/mL.

22. The method of any one of claims 17-18, and 21, wherein the viable cell density of the host cells in the physiologically compatible fluid ranges from about 10×10⁶ vc/mL to 30×10⁶ vc/mL, the detergent is Triton X-100, the final concentration of Triton X-100 in the lysate ranges from about 0.3% to 0.7%, or from about 0.35% to 0.65%, or from about 0.4% to 0.6%; and the final concentration of DB in the lysate ranges from about 0.15% to 0.45%, or from about 0.2% to 0.4%, or from about 0.2% to 0.3%.

23. The method of any one of claims 17-18, and 21-22, wherein the viable cell density of the host cells in the physiologically compatible fluid ranges from about 15×10⁶ vc/mL to 25×10⁶ vc/mL, the detergent is Triton X-100, the final concentration of Triton X-100 in the lysate ranges from about 0.3% to 0.7%, or from about 0.35% to 0.65%, or from about 0.4% to 0.6%; and the final concentration of DB in the lysate ranges from about 0.15% to 0.45%, or from about 0.2% to 0.4%, or from about 0.2% to 0.3%.

24. The method any one of claims 17-18, and 21-23, wherein the final concentration of Triton X-100 is about 0.5%, and the final concentration of DB is about 0.3%.

25. The method of any one of claims 1-12, 15-18, and 21-24, wherein the supernatant is separated from the flocculant by settling under the influence of gravity, forming a lower layer of settled flocculant and an upper layer of supernatant.

26. The method of any one of claims 1-12, 15-18, and 21-25, further comprising removing and filtering the supernatant.

27. The method of any one of claims 1-12, 15-18, and 21-26, wherein precipitation of residual host cell DNA in the supernatant is inhibited by adding to the supernatant a solution comprising a salt in a concentration sufficient to inhibit precipitation of host cell DNA.

28. The method of claim 27, wherein the salt is sodium chloride (NaCl), potassium chloride (KCl), magnesium sulfate (MgSO₄), or magnesium chloride (MgCl₂).

29. The method of any one of claims 27-28, further comprising mixing the supernatant and salt solution.

30. The method of any one of claims 27-29, wherein prior to lysis, the viable cell density of the host cells is at least about 10×10⁶ vc/mL, the final concentration of DB in the lysate is at least about 0.2%, the salt is MgSO₄ or MgCl₂, and the final concentration of the added salt in the supernatant is at least about 10 mM.

31. The method of any one of claims 27-29, wherein prior to lysis, the viable cell density of the host cells is at least about 10×10⁶ vc/mL, the final concentration of DB in the lysate is at least about 0.2%, the salt is NaCl or KCl, and the final concentration of the added salt in the supernatant is at least about 100 mM.

32. The method of any one of claims 17-31, wherein the final concentration of DB in the lysate relative to the viable cell density prior to lysis is not less than 0.007% per 1×10⁶ vc/mL.

33. The method of any one of claims 28-32, wherein the viable cell density of the host cells in the physiologically compatible fluid ranges from about 10×10⁶ vc/mL to 30×10⁶ vc/mL, the final concentration of DB in the lysate ranges from about 0.2% to 0.4%, or from about 0.2% to 0.3%, the salt is NaCl or KCl, and the final concentration of the added salt in the supernatant is at least about 100 mM, or at least about 200 mM, or ranges from about 200 mM to about 700 mM.

34. The method of any one of claims 28-33, wherein the viable cell density of the host cells in the physiologically compatible fluid ranges from about 15×10⁶ vc/mL to 25×10⁶, the final concentration of DB in the lysate ranges from about 0.2% to 0.4%, or from about 0.2% to 0.3%, the salt is NaCl or KCl, and the final concentration of the added salt in the supernatant is at least about 100 mM, or at least about 200 mM, or ranges from about 200 mM to about 700 mM.

35. The method of any one of claims 27-34, wherein the detergent is Triton X-100 and the final concentration of Triton X-100 in the lysate is at least about 0.3%, or ranges from about 0.3% to 0.7%, or from 0.35% to 0.65%, or from 0.4% to 0.6%, or is about 0.5%.

36. The method of claim 35, wherein the final concentration of Triton X-100 is about 0.5%, and the final concentration of DB is about 0.3%.

37. The method of any one of claims 29-36, further comprising filtering the mixture of the supernatant and salt solution.

38. The method of claim 37, further comprising purifying the biological product by performing a downstream purification processing step.

39. The method of claim 38, wherein the mixture of the supernatant and salt solution is held for at least 3 hours before performing the downstream purification processing step.

40. The method of any one of claims 1-39, wherein the biological product is a recombinant viral vector for expressing a heterologous gene.

41. The method of claim 40, wherein the recombinant viral vector is an adenovirus vector, adeno-associated virus (AAV) vector, retrovirus vector, or lentivirus vector.

42. The method of claim 41, wherein the recombinant viral vector is an AAV vector.

43. The method of claim 42, wherein the AAV vector comprises a capsid that binds more strongly to sialic acid or galactose as compared to HSPG.

44. The method of any one of claims 42-43, wherein the AAV vector comprises an AAV1, AAV4, AAV5, or AAV9 capsid.

45. The method of any one of claims 42-44, wherein the downstream purification processing step comprises chromatography.

46. The method of claim 45, wherein the method is effective to produce an AAV vector yield of at least 50%, 60%, or 70% after at least 5, 6, 7, 8, 9, 10, 11, 12, 13, 14, 15 or more chromatography purification cycles.

47. The method of claim 46, wherein the method is effective to produce an AAV vector yield of at least 50% after at least 5 chromatography purification cycles.

48. The method of any one of claims 45-47, wherein the chromatography is affinity chromatography, pseudoaffinity chromatography, anion exchange chromatography, cation exchange chromatography, hydrophobic interaction chromatography, or size exclusion chromatography.

49. The method of claim 48, wherein the affinity chromatography is immunoaffinity chromatography.

50. The method of any one of claims 1-49, wherein no endonuclease is added to the lysate.

51. The method of any one of claims 1-50, wherein no salt is added prior to the step of separating the supernatant from the flocculant.

52. The method of any one of claims 1-51, wherein the volume of the cell suspension prior to lysis is at least 100 L.

53. A biological product produced by the method of any one of claims 1-52.

54. The biological product of claim 53, wherein said biological product is a recombinant viral vector for expressing a heterologous gene selected from the group consisting of: an adenovirus vector, an adeno-associated virus (AAV) vector, a retrovirus vector, or a lentivirus vector.

55. The biological product of claim 54, wherein said biological product is an AAV vector.

56. A composition comprising the AAV vector of claim 55.

57. The composition of claim 56, wherein the capsids in said AAV vector composition are at least 20%, 30%, 40%, 50%, 60%, 70%, 80%, or 90% full capsids.

58. The composition of any one of claims 56-57, wherein said composition comprises not more than about 200, 150, 100, 90, 80, 70, 60, 50, 45, 40, 35, 30, 25, or 20 pg/1×10⁹ vg of host cell DNA.
